(19) United States
(12) Patent Application Publication
(10) Pub. No.: US 2024/0325495 A1
(43) Pub. Date: Oct. 3, 2024

(54) FUSION PROTEINS FOR THE TREATMENT OF DISEASE

(71) Applicant: Bristol-Myers Squibb Company, Princeton, NJ (US)

(72) Inventors: Alice L. WANG, Boston, MA (US); Mary STRUTHERS, Edison, NJ (US); Kimberly MCGORMAN, Princeton, NJ (US); Neelanjana RAY, Princeton, NJ (US); Karen D. PRICE, Princeton, NJ (US); Nidhi SHARDA, Princeton, NJ (US); Eun Mi HUR, Princeton, NJ (US); Priyanka Apurva MADIA, Franklin Park, NJ (US)

(73) Assignee: Bristol-Myers Squibb Company, Princeton, NJ (US)

(21) Appl. No.: 18/251,214

(22) PCT Filed: Oct. 29, 2021
(86) PCT No.: PCT/US2021/057352

§ 371 Date: Apr. 28, 2023

Related U.S. Application Data
(60) Provisional application No. 63/198,615, filed on Oct. 29, 2020.
(60) Provisional application No. 63/123,991, filed on Dec. 10, 2020.

(51) Int. Cl. A61K 38/20; A61K 31/573; A61K 38/17; A61P 37/06

(52) U.S. Cl. CPC A61K 38/2013; A61K 31/573; A61K 38/1793; A61P 37/06

(57) ABSTRACT

Disclosed herein are methods of treating a disease or disorder in a subject in need thereof, comprising administering to the subject one or more doses of an Interleukin-2 (IL2) fusion protein, wherein the dose is from about 0.1 mg to about 9 mg. Also disclosed herein are methods of treating a disease or disorder in a subject in need thereof, comprising administering to the subject an dose of an IL2 fusion protein, wherein the dose is greater than about 9 mg. The IL2 fusion proteins used in the methods disclosed herein comprise: (a) a first polypeptide comprising an IL2 polypeptide; and (b) a second polypeptide comprising an extracellular domain of an IL2 Receptor alpha (IL2R) polypeptide. In some aspects, the disease or disorder is an immune-mediated disease, such as systemic lupus erythematosus. In some aspects, the methods further administering a corticosteroid to the subject.

### CROSS-REFERENCE TO RELATED APPLICATIONS

This PCT application claims the benefit of U.S. Provisional Application No. 63/198,615, filed Oct. 29, 2020 and U.S. Provisional Application No. 63/123,991, filed Dec. 10, 2020, both of which are incorporated herein by reference in their entireties.

### REFERENCE TO SEQUENCE LISTING SUBMITTED ELECTRONICALLY

The content of the electronically submitted sequence listing in ASCII text file (Name: 3338.234PC02_SL_ST25.txt; Size: 49,540 bytes; and Date of Creation: Oct. 29, 2021) filed with the application is incorporated herein by reference in its entirety.

### FIELD OF THE DISCLOSURE

The present disclosure provides methods for treating a disease or disorder in a subject by administering one or more doses of an Interleukin-2 (IL2)/IL2 Receptor a fusion protein.

### BACKGROUND OF THE DISCLOSURE

Interleukin-2 (IL2 or IL-2) is a biologic cytokine that regulates key aspects of the immune system. IL-2 has been used in attempts to boost immune responses in patients with inflammatory disease or an autoimmune disease. IL-2 is a potent T cell growth factor that promotes immune responses, including clonal expansion of antigen-activated T cells, drives development of CD4+T-helper (Th)1 and Th2 cells, terminally differentiates CD8+ cytotoxic T lymphocytes (CTLs), and opposes development of CD4+Th17 and T-follicular helper (Tfh) cells. IL-2 also shapes T cell memory recall responses.

The importance of the IL-2 signaling pathway on Tregs has been demonstrated by the appearance of systemic autoimmunity in mice or humans lacking components of the IL-2 signaling pathway. Dysregulation of regulatory T cell (Treg) numbers and/or function has been implicated in numerous immune-mediated conditions. See, for example, Bluestone, J. A., et al., J Clin Invest. 125:2250-60 (2015); and Dominguez-Villar, M and Hafler, D. A., Nat Immunol. 19:665-73 (2018). Autoimmune risk variants in the IL-2, IL-2Rα, and IL-2Rβ loci have been identified through genome-wide association studies (GWAS) and associated with immune-mediated diseases including inflammatory bowel disease (IBD), Type-1 autoimmune diabetes (T1DM), multiple sclerosis (MS), and rheumatoid arthritis (RA). See, for example, Abbas, A. K., et al., Sci Immunol. 3, eaat1482 (2018). Mutations affecting the key Treg lineage transcription factor FoxP3 cause the autoimmune lymphoproliferative disease Immune Dysregulation, Polyendocrinopathy, enteropathy, X-linked (IPEX) syndrome, resulting from loss of functional Treg. In addition, patients with CD25 deficiency, which results from mutations in IL-2RA, suffer from immune dysregulation similar to IPEX syndrome. See, for example, Verbsky, J. W. and Chatila, T., Curr Opin Pediatr. 25(6):708-14 (2013). Genetic data are consistent with a central role for IL-2 in Treg function and suppression of autoimmunity in both mice and humans.

Because of the importance of the effect of IL-2 on Tregs, low-dose recombinant IL-2 has been used for Treg-based immunosuppressive strategies in immune-mediated diseases. See, for example, Saadoun, D., et al., N Engl J Med. 365:2067-77 (2011); He, J., et al., Arthritis Rheumatol. 67(suppl 10) (2015); Koreth, J., et al., N Engl J Med. 365:2055-66 (2011); and Humrich, J. Y., et al. Ann Rheum Dis. 74:791-2 (2015). For example, systemic lupus erythematosus (SLE) is characterized by an IL-2 deficient state, with Tregs showing diminished immune regulatory capacity. A low-dose of IL-2 has shown encouraging clinical benefits in SLE patients; however, its clinical utility is limited due to the requirement of daily injections and the observation of increases in pro-inflammatory cytokines and in non-Treg cells. In contrast, high-dose IL-2 has been used to stimulate an anti-tumor immune response via T effector cells. See, for example, Rosenberg, S. A., J Immunol. 192:5451-8 (2014).

Despite the promising results from these clinical studies, low-dose recombinant IL-2 therapy is limited by a very short half-life (minutes), necessitating frequent dosing, and a small window to activation of non-Treg effects potentially limiting efficacy. Thus, there remains a need for new IL2 biologics having improved pharmacokinetics and durability of responses for use, for example in the treatment of infectious disease and immune-mediated disease such as SLE.

### SUMMARY OF THE DISCLOSURE

Certain aspects of the present disclosure are directed to a method of treating a disease or disorder in a subject in need thereof, comprising administering to the subject one or more doses of an Interleukin-2 (IL2) fusion protein, wherein the fusion protein comprises: (a) a first polypeptide comprising an IL2 polypeptide, and (b) a second polypeptide comprising an extracellular domain of an Interleukin-2 Receptor alpha (IL2Rα) polypeptide; wherein (i) the extracellular domain of the IL2Rα polypeptide has at least one fewer glycosylation compared to the extracellular domain of native IL2Rα (SEQ ID NO: 1); and/or (ii) the IL2 polypeptide has at least one fewer glycosylation compared to native IL2 (SEQ ID NO: 2); wherein one or more of the doses are from about 0.1 mg to about 9 mg.

In some aspects, the fusion protein is administered to the subject via a topical, epidermal, mucosal, intranasal, oral, vaginal, rectal, sublingual, topical, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural or intrasternal route.

In some aspects, the fusion protein is administered to the subject via an intravenous route. In some aspects, the fusion protein is administered via an intravenous route at a dose of between about 0.3 mg to about 9 mg.

In some aspects, the fusion protein is administered to the subject via an intravenous route at a dose of between about 1 mg and about 9 mg, between about 2 mg and about 9 mg, between about 3 mg and about 9 mg, between about 4 mg and about 9 mg, between about 5 mg and about 9 mg, between about 6 mg and about 9 mg, between about 7 mg and about 9 mg, between about 8 mg and about 9 mg, between about 1 mg and about 8 mg, between about 2 mg and about 8 mg, between about 3 mg and about 8 mg, between about 4 mg and about 8 mg, between about 5 mg and about 8 mg, between about 6 mg and about 8 mg, between about 7 mg and about 8 mg, between about 1 mg and about 7 mg, between about 2 mg and about 7 mg, between about 3 mg and about 7 mg, between about 4 mg and about 7 mg, between about 5 mg and about 7 mg, between about 6 mg and about 7 mg. In some aspects, the dose administered via an intravenous route is between about 3 mg and about 9 mg. In some aspects, the dose administered via an intravenous route is between about 6 mg and about 9 mg.

In some aspects, the fusion protein is administered to the subject via an intravenous route at a dose of between about 0.1 mg and about 6 mg, between about 1 mg and about 6 mg, between about 2 mg and about 6 mg, between about 3 mg and about 6 mg, between about 4 mg and about 6 mg, or between about 5 mg and about 6 mg, between about 1 mg and about 5 mg, between about 2 mg and about 5 mg, between about 3 mg and about 5 mg, between about 4 mg and about 5 mg, between about 1 mg and about 4 mg, between about 2 mg and about 4 mg, between about 3 mg and about 4 mg, between about 1 mg and about 3 mg, or between about 2 mg and about 3 mg. In some aspects, the dose administered via an intravenous route is between about 0.1 mg and about 3 mg. In some aspects, the dose administered via an intravenous route is between about 0.1 mg and about 1 mg. In some aspects, the dose administered via an intravenous route is between about 0.1 mg and about 0.3 mg. In some aspects, the dose administered via an intravenous route is between about 0.3 mg and about 6 mg. In some aspects, the dose administered via an intravenous route is between about 1 mg and about 3 mg.

In some aspects, the dose administered via an intravenous route is about 0.1 mg, about 0.3 mg, about 1 mg, about 2 mg, about 3 mg, about 4 mg, about 5 mg, about 6 mg, about 7 mg, about 8 mg, or about 9 mg.

In some aspects, the dose administered via an intravenous route is greater than about 9 mg.

In some aspects, the fusion protein is administered to the subject via a subcutaneous route. In some aspects, the fusion protein is administered to the subject via a subcutaneous route at a dose of between about 1 mg and about 8 mg, between about 2 mg and about 8 mg, between about 3 mg and about 8 mg, between about 4 mg and about 8 mg, between about 5 mg and about 8 mg, between about 6 mg and about 8 mg, between about 7 mg and about 8 mg, between about 1 mg and about 7 mg, between about 2 mg and about 7 mg, between about 3 mg and about 7 mg, between about 4 mg and about 7 mg, between about 5 mg and about 7 mg, between about 6 mg and about 7 mg, between about 1 mg and about 6 mg, between about 2 mg and about 6 mg, between about 3 mg and about 6 mg, between about 4 mg and about 6 mg, or between about 5 mg and about 6 mg, between about 1 mg and about 5 mg, between about 2 mg and about 5 mg, between about 3 mg and about 5 mg, between about 4 mg and about 5 mg, between about 1 mg and about 4 mg, between about 2 mg and about 4 mg, between about 3 mg and about 4 mg, between about 1 mg and about 3 mg, or between about 2 mg and about 3 mg. In some aspects, the dose administered via a subcutaneous route is between about 3 mg and about 8 mg. In some aspects, the dose administered via a subcutaneous route is between about 6 mg and about 8 mg. In some aspects, the dose administered via a subcutaneous route is between about 1 mg to about 6 mg. In some aspects, the dose administered via a subcutaneous route is between about 1 mg to about 3 mg. In some aspects, the dose administered via a subcutaneous route is between about 3 mg to about 6 mg.

In some aspects, the dose administered via subcutaneous route is about 1 mg, about 3 mg, about 6 mg, or about 8 mg.

In some aspects, the dose administered via subcutaneous route is greater than about 8 mg.

In some aspects, the method includes administering two or more of the dose of the fusion protein at a dosing interval between two doses of the fusion protein. In some aspects, the dosing interval of the fusion protein is at least about one day, at least about two days, at least about three days, at least about four days, at least about five days, or at least about six days. In some aspects, the dosing interval of the fusion protein is at least about one week, at least about two weeks, at least about three weeks, at least about four weeks, at least about a month, at least about five weeks, at least about six weeks, at least about seven weeks, at least about eight weeks, at least about two months, at least about nine weeks, at least about ten weeks, at least about eleven weeks, at least about twelve weeks, or at least about three months. In some aspects, the dosing interval is at least about three weeks. In some aspects, the dosing interval of the fusion protein is about one day, about two days, about three days, about four days, about five days, or about six days. In some aspects, the dosing internal of the fusion protein is about a week, about two weeks, about three weeks, about four weeks, about a month, about five weeks, about six weeks, about seven weeks, about eight weeks, about two months, about nine weeks, about 10 weeks, about 11 weeks, about 12 weeks, or about three months. In some aspects, the dosing interval of the fusion protein is about three weeks. In some aspects, the dosing interval of the fusion protein is the same throughout the doses. In some aspects, the dosing interval of the fusion protein is different throughout the doses. In some aspects, at least one of the two or more doses of the fusion protein is administered intravenously and at least one of the two or more doses of the fusion protein is administered subcutaneously. In some aspects, the dose administered intravenously is given before the dose administered subcutaneously. In some aspects, the first dose of the fusion protein is administered intravenously and the second (any subsequent or final) dose of the fusion protein is administered subcutaneously.

In some aspects, the disease or disorder is an infectious disease, an immune-mediated disease. In some aspects, the immune-mediated disease is an inflammatory disease or an autoimmune disease. In some aspects, the immune-mediated disease is selected from the group consisting of: type 1 diabetes; multiple sclerosis; rheumatoid arthritis; celiac disease; systemic lupus erythematosus; lupus nephritis; cutaneous lupus; juvenile idiopathic arthritis; Crohn's disease; ulcerative colitis; systemic sclerosis; graft versus host disease (GvHD); psoriasis; alopecia areata; HCV-induced vasculitis; Sjogren's syndrome; Pemphigus; Ankylosing Spondylitis; Behcet's Disease; Wegener's Granulomatosis; Takayasu's Disease; Autoimmune Hepatitis; Sclerosing Cholangitis; Gougerot-sjögren; inflammatory bowel disease; Immune Dysregulation, Polyendocrinopathy, Enteropathy, X-linked (IPEX) syndrome; and Macrophage Activation Syndrome. In some aspects, the immune-mediated disease is systemic lupus erythematosus, lupus nephritis, or cutaneous lupus. In some aspects, the immune-mediated disease is systemic lupus erythematosus.

In some aspects, the method further comprises administering to the subject a corticosteroid. In some aspects the corticosteroid is selected from the group consisting of: prednisolone, methylprednisolone, prednisone, hydrocortisone, dexamethasone, betamethasone, budesonide, triamcinolone, cortisone, desoxycorticosterone, fludrocortisone, and paramethasone. In some aspects, the corticosteroid is prednisolone, methylprednisolone, or prednisone. In some aspects, the corticosteroid is prednisolone.

In some aspects, the corticosteroid is administered to the subject via a topical, epidermal, mucosal, intranasal, oral, vaginal, rectal, sublingual, topical, intravenous, intraperitoneal, intramuscular, inaarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural, or intrasernal route. In some aspects, the corticosteroid is administered to the subject via a topical, oral, intravenous, or intramuscular route.

In some aspects, the corticosteroid is administered before, concurrently with, or after each dose of the of the fusion protein. In some aspects, the corticosteroid is administered before each dose of the of the fusion protein. In some aspects, the corticosteroid is administered concurrently with each dose of the of the fusion protein. In some aspects, two or more doses of the corticosteroid are administered to the subject at a dosing interval between each dose. In some aspects, the dosing interval of the corticosteroid is at least about one day, at least about two days, at least about three days, at least about four days, at least about five days, at least about six days, at least about one week, at least about two weeks, at least about three weeks, at least about four weeks, at least about a month, at least about five weeks, at least about six weeks, at least about seven weeks, at least about eight weeks, at least about two months, at least about nine weeks, at least about ten weeks, at least about eleven weeks, at least about twelve weeks, or at least about three months. In some aspects, the corticosteroid is prednisoloine, wherein the fusion protein is administered to the subject subcutaneously twice a week, and wherein the prednisolone is administered to the subject orally three times a week.

In some aspects, the extracellular domain of the IL2Rα polypeptide has at least one fewer glycosylation, at least two fewer glycosylations, at least three fewer glycosylations, at least four fewer glycosylations, at least five fewer glycosylations, at least six fewer glycosylations, at least seven fewer glycosylations, at least eight fewer glycosylations, or at least nine fewer glycosylations compared to the extracellular domain of native IL2Rα (SEQ ID NO: 1).

In some aspects, the IL2 polypeptide has at least one fewer glycosylation compared to native IL2 (SEQ ID NO: 2).

In some aspects, the first polypeptide comprises an amino acid sequence at least about 60%, at least about 70%, at least about 80%, at least about 85%, at least about 90%, at least about 95%, at least about 96%, at least about 97%, at least about 98%, at least about 99% or about 100% identical to SEQ ID NO: 2.

In some aspects, the second polypeptide comprises an amino acid sequence at least about 60%, at least about 70%, at least about 80%, at least about 85%, at least about 90%, at least about 95%, at least about 96%, at least about 97%, at least about 98%, at least about 99%, or about 100% identical to SEQ ID NO: 3.

In some aspects, the extracellular domain of the IL2Rα polypeptide having at least one fewer glycosylation comprises a mutation that removes a glycosylation. In some aspects, the mutation removes an O-glycosylation and/or an N-glycosylation. In some aspects, the mutation removes an O-glycosylation. In some aspects, the mutation removes an N-glycosylation. In some aspects, the mutation is a deletion of amino acids 167 to 219, amino acids 168 to 219, amino acids 169 to 219, amino acids 170 to 219, amino acids 171 to 219, amino acids 172 to 219, amino acids 173 to 219, amino acids 174 to 219, amino acids 175 to 219, amino acids 176 to 219, amino acids 177 to 219, amino acids 178 to 219, amino acids 179 to 219, amino acids 180 to 219, amino acids 181 to 219, amino acids 182 to 219, amino acids 183 to 219, amino acids 184 to 219, amino acids 185 to 219, amino acids 186 to 219, amino acids 187 to 219, amino acids 188 to 219, amino acids 189 to 219, amino acids 190 to 219, amino acids 191 to 219, or amino acids 192 to 219, corresponding to SEQ ID NO: 1.

In some aspects, the second polypeptide is SEQ ID NO: 4. In some aspects, the second polypeptide is SEQ ID NO: 3.

In some aspects, the mutation is one or more substitutions of an amino acid that is glycosylated with an amino acid that is not glycosylated. In some aspects, the one or more substitutions are at amino acid N49, amino acid N68, amino acid T74, amino acid T85, amino acid T197, amino acid T203, amino acid T208, and amino acid T216, or any combination thereof, wherein the amino acid locations correspond to SEQ ID NO: 1. In some aspects, the one or more substitutions are from threonine to an amino acid selected from the group consisting of alanine, arginine, asparagine, aspartic acid, cysteine, glutamine, glutamic acid, glycine, histidine, isoleucine, leucine, lysine, methionine, phenylalanine, proline, serine, tryptophan, tyrosine, and valine.

In some aspects, one of the substitutions is amino acid T85. In some aspects, T85 is mutated to an amino acid selected from the group consisting of alanine, arginine, asparagine, aspartic acid, cysteine, glutamine, glutamic acid, glycine, histidine, isoleucine, leucine, lysine, methionine, phenylalanine, proline, serine, tryptophan, tyrosine, and valine.

In some aspects, one of the substitutions is amino acid T197. In some aspects, T197 is mutated to an amino acid selected from the group consisting of alanine, arginine, asparagine, aspartic acid, cysteine, glutamine, glutamic acid, glycine, histidine, isoleucine, leucine, lysine, methionine, phenylalanine, proline, serine, tryptophan, tyrosine, and valine.

In some aspects, one of the substitutions is amino acid T203. In some aspects, T203 is mutated to an amino acid selected from the group consisting of alanine, arginine, asparagine, aspartic acid, cysteine, glutamine, glutamic acid, glycine, histidine, isoleucine, leucine, lysine, methionine, phenylalanine, proline, serine, tryptophan, tyrosine, and valine.

In some aspects, one of the substitutions is amino acid T208. In some aspects, T208 is mutated to an amino acid selected from the group consisting of alanine, arginine, asparagine, aspartic acid, cysteine, glutamine, glutamic acid, glycine, histidine, isoleucine, leucine, lysine, methionine, phenylalanine, proline, serine, tryptophan, tyrosine, and valine.

In some aspects, one of the substitutions is amino acid T216. In some aspects, T216 is mutated to an amino acid selected from the group consisting of alanine, arginine, asparagine, aspartic acid, cysteine, glutamine, glutamic acid, glycine, histidine, isoleucine, leucine, lysine, methionine, phenylalanine, proline, serine, tryptophan, tyrosine, and valine.

In some aspects, the mutation is one or more substitutions of an amino acid that allows glycosylation at a nearby amino acid with an amino acid that does not allow glycosylation at the nearby amino acid. In some aspects, the one or more substitutions are at amino acid S50, amino acid S51, amino acid T69, amino acid T70, amino acid C192, or any combination thereof, wherein the amino acid locations correspond to SEQ ID NO: 1.

In some aspects, one of the substitutions is at amino acid S50. In some aspects, S50 is mutated to proline.

In some aspects, one of the substitutions is at amino acid S51. In some aspects, S51 is mutated to an amino acid selected from the group consisting of alanine, arginine, asparagine, aspartic acid, cysteine, glutamine, glutamic acid, glycine, histidine, isoleucine, leucine, lysine, methionine, phenylalanine, proline, serine, tryptophan, tyrosine, and valine.

In some aspects, one of the substitutions is at amino acid T69. In some aspects, T69 is mutated to proline.

In some aspects, one of the substitutions is at amino acid T70. In some aspects, T70 is mutated to an amino acid selected from the group consisting of alanine, arginine, asparagine, aspartic acid, cysteine, glutamine, glutamic acid, glycine, histidine, isoleucine, leucine, lysine, methionine, phenylalanine, proline, threonine, tryptophan, tyrosine, and valine.

In some aspects, one of the substitutions is at amino acid C192. In some aspects, C192 is mutated to an amino acid selected from the group consisting of alanine, arginine, asparagine, aspartic acid, glutamine, glutamic acid, glycine, histidine, isoleucine, leucine, lysine, methionine, phenylalanine, proline, serine, threonine, tryptophan, tyrosine, and valine.

In some aspects, the IL2 polypeptide having at least one fewer glycosylation comprises a mutation that removes a glycosylation. In some aspects, the mutation is one or more substitutions of an amino acid that is glycosylated with an amino acid that is not glycosylated. In some aspects, the mutation is one or more substitutions of an amino acid that allows glycosylation at a nearby amino acid with an amino acid that does not allow glycosylation at the nearby amino acid. In some aspects, the one or more substitutions are from an alanine to an amino acid selected from the group consisting of arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine. In some aspects, the one or more substitutions are from a threonine to an amino acid selected from the group consisting of alanine, arginine, asparagine, aspartic acid, cysteine, glutamine, glutamic acid, glycine, histidine, isoleucine, leucine, lysine, methionine, phenylalanine, proline, tryptophan, tyrosine, and valine. In some aspects, the one or more substitutions are from a cysteine to an amino acid selected from the group consisting of alanine, arginine, asparagine, aspartic acid, glutamine, glutamic acid, glycine, histidine, isoleucine, leucine, lysine, methionine, phenylalanine, proline, serine, threonine, tryptophan, tyrosine, and valine. In some aspects, the one or more substitutions are from a cysteine to a serine. In some aspects, the one or more substitutions are from a cysteine to an alanine. In some aspects, the one or more substitutions are from a cysteine to a valine. In some aspects one of the substitutions is at amino acid T3 compared to corresponding to SEQ ID NO: 2. In some aspects, one of the substitutions is at amino acid C125, wherein the substitution at amino acid C125 is selected from the group consisting of C125S, C125A, and C125V. In some aspects, the mutation is a deletion. In some aspects, the deletion is at amino acid A1.

In some aspects, the fusion protein further comprises a linker fused in frame between the first polypeptide and the second polypeptide. In some aspects, the linker is a glycine/serine linker. In some aspects, the glycine/serine linker comprises an amino acid sequence of (GS)ₙ, (GGS)ₙ, (GGGS)ₙ, (GGGGS)ₙ, or (GGGGS)ₙ, wherein n is an integer of 1, 2, 3, 4, 5, 6, 7, 8, 9, or 10. In some aspects, the glycine/serine linker comprises the amino acid sequence of (GGGS)₃.

In some aspects, the fusion protein comprises the amino acid sequence as set forth in SEQ ID NO: 5.

In some aspects, the fusion protein further comprises a heterologous moiety fused to the first polypeptide and/or the second polypeptide. In some aspects, the heterologous moiety is a half-life extending moiety. In some aspects, the heterologous moiety comprises albumin, an immunoglobulin constant region or a portion thereof, an immunoglobulin-binding polypeptide, an immunoglobulin G (IgG), albumin-binding polypeptide (ABP), a PASylation moiety, a HESylation moiety, XTEN, a PEGylation moiety, an Fc region, and any combination thereof.

In some aspects, the fusion protein consists of the amino acid sequence as set forth in SEQ ID NO: 5.

In some aspects, the fusion protein is deglycosylated enzymatically or chemically. In some aspects, the fusion protein is deglycosylated by alkali, hydrazinolysis, PNGase F, Endo H, O-glycosidase, or any combination thereof.

In some aspects, the fusion protein is a monomer. In some aspects, the fusion protein is a dimer.

In some aspects, the fusion protein is administered to the subject as part of a pharmaceutical composition comprising the fusion protein and a pharmaceutically acceptable excipient.

### BRIEF DESCRIPTION OF THE DRAWINGS

FIGS. 1A and 1B show a schematic diagram of the Phase 1, randomized, double-blind, placebo-controlled, single ascending doses (“SAD”) study design (FIG. 1A) and the single ascending dosing regimen (FIG. 1B) described in Example 1. Sentinel dosing (1:1 BMS-986326 or placebo) is used in all cohorts. Dose levels may change depending on emerging data from a prior cohort. The maximum dose-escalation step will be approximately ≤3-fold the previous dose level. “IV NOAEL^{a}” denotes the maximum intravenous (IV) dose that is anticipated to provide a mean exposure (area under the concentration-time curve from time zero to infinity; “AUC[INF]”) to the NOAEL (AUC[0-336 hours]≤757 μ·h/ml) for the single-dose monkey toxicology study. Cohort A6 is optional, depending on the pharmacodynamic (PD) results from prior cohorts. “SC^{b}” denotes the maximum subcutaneous (SC) dose that is anticipated to provide a mean exposure (AUC[INF]) that will not exceed the NOAEL (AUC[0-504 hours]≤306 μ·h/ml) for the 12-week (once every 3 weeks) SC monkey toxicology study.

FIGS. 2A-2B show Treg CD25 expression in NZB×NZQ versus BALB/c mice. Splenocytes from NZB×NZW (n=5, 26 weeks of age) or BALB/c (n=6, 9-10 weeks of age) mice were stained with antibodies to CD4, Foxp3, and CD25. FIG. 2A shows the percentage of Foxp3⁺ cells in CD4⁺ gate and the percentage of CD25⁺ cells in CD4⁺Fox3⁺ gate from a representative mouse of each group. FIG. 2B shows mean fluorescence intensity (MFI) of the CD4⁺Foxp3⁺ T cells (Mean±SEM). ***p<0.001 by one-way ANOVA.

FIGS. 3A-3E show the effect of mIL-2/CD25 administration on Tregs and non-Treg cells in BALB/c mice. BALB/c mice (n=5, 8-10 weeks of age) were treated with mIL-2/CD25 on days 0, 3, and 6, with Fc-IL2 on days 0, 2, 4, and 6, or PBS on days 0, 3, and 6. Mice were sacrificed on day 7, and spleens were harvested for analysis by flow cytometry. FIG. 3A shows the percentage of Foxp3⁺CD25⁺ cells among CD4⁺ T cells. FIG. 3B shows the total number of CD4⁺Foxp3+CD25⁺ T cells. FIG. 3C shows the total number of CD4⁺Foxp3⁻ T cells. FIG. 3D shows the total number of CD8⁺ T cells. FIG. 3E shows the total number of CD335⁺CD49d⁺ NK cells. *p<0.05, **p<0.01, ***p<0.001, ****p<0.0001 vs. PBS control group by one-way ANOVA.

FIGS. 4A-4D show inhibition of lupus by mIL-2/CD25 administration in NZB×NZW mice (early disease). Female NZB×NZW F1 mice (22-24 weeks of age) with proteinuria level of 30 mg/dL were treated for 14 weeks with PBS or mIL-2/CD25 at 0.1, 0.2, or 0.4 mg/kg (s.c. 2×/week), or with Prednisolone (10 mg/kg p.o. 3×/week) with n=10 per group. mIL-2/CD25 administration showed a dose dependent reduction in the levels of proteinuria (FIG. 4A), the percentage of mice with high proteinuria (score of 3 or higher) (FIG. 4B), the anti-dsDNA IgG titers (FIG. 4C), and the kidney histological scores (FIG. 4D). **p<0.01, ****p<0.0001 vs PBS group at the end of the study by one-way ANOVA.

FIGS. 5A-5D show the effect of mIL-2/CD25 administration on Tregs in NZB×NZW mice. Female NZB×NZW F1 mice (22-24 weeks of age) with proteinuria level of 30 mg/dL were treated for 4 weeks with PBS or mIL-2/CD25 at 0.1, 0.2 or 0.4 mg/kg (s.c. 2×/week), or with Prednisolone (p.o. 3×/week) with n=4 per group. mIL-2/CD25 (48 h post 8th dose) showed a dose dependent increase in the percentage of Tregs (CD4+CD25+Foxp3+) in blood (FIG. 5A) and the spleen (FIG. 5B). The percentage of Ki67⁺ cells (FIG. 5C) and CD25 MFI (FIG. 5D) in the Treg gate (CD4⁺CD25⁺Foxp3⁺) were also increased in a dose dependent fashion in the spleen. *p<0.05, **p<0.01, ***p<0.001, ****p<0.0001 vs. PBS group by one-way ANOVA.

FIGS. 6A-6E show inhibition of lupus by mIL-2/CD25 administration in NZB×NZW mice (advanced disease). Female NZB×NZW F1 mice (˜27 weeks of age) with proteinuria levels of 100 mg/dL were treated for 10 weeks with PBS or mIL-2/CD25 at 0.1 or 0.3 mg/kg (s.c. 2×/week), with n=12-14 per group. Treatment with mIL-2/CD25 resulted in a dose dependent reduction in the levels of proteinuria (FIG. 6A), a trend towards reduction in the anti-dsDNA IgG titers (FIG. 6B), a reduction in the levels of serum IL-12 (FIG. 6C), a reduction in the kidney histological scores (FIG. 6D), and an increase in the percentage of CD4⁺Foxp3⁺CD25⁺ cells in the spleen (FIG. 6E) at completion of the study. *p<0.05 vs PBS group by one-way ANOVA. #p<0.05 vs PBS group by two-tailed t-test.

FIGS. 7A-7E show the effect of mIL-2/CD25 and Prednisolone combination treatment in NZB×NZW mice. Female NZB×NZW F1 mice (21-23 weeks of age) with proteinuria level of 30 mg/dL were treated for 14 weeks with either PBS, mIL-2/CD25 (0.1 mg/kg s.c. 2×/week), prednisolone (1 mg/kg p.o. 3×/week), or with combination of mIL-2/CD25 (0.1 mg/kg s.c. 2×/week) and prednisolone (1 mg/kg p.o. 3×/week). A high dose of prednisolone (10 mg/kg, p.o. 3×/week) and mIL-2/CD25 (0.2 ug/kg s.c. 2×/week) groups were included as controls, n=12 per group. The effect on the levels of proteinuria (FIG. 7A), the anti-dsDNA antibody titers (FIG. 7B), and the histological scores (FIG. 7C) are shown. mIL-2/CD25 treatment showed an increase in the percentage of Tregs (CD4⁺CD25⁺Foxp3⁺) (FIG. 7D) and the CD25 MFI in Treg gate (CD4⁺CD25⁺Foxp3⁺) (FIG. 7E) in the spleen, with or without the presence of prednisolone. n=5 per group for FIG. 7D and FIG. 7E. *p<0.05, **p<0.01, ***p<0.001, ****p<0.0001 vs. PBS group at the end of the study by one-way ANOVA.

FIGS. 8A-8F show the effect of mIL-2/CD25 and Prednisolone combination treatment in NZB×NZW mice. At completion of the mIL-2/CD25 and Prednisolone combination study as described in FIG. 7, kidneys were collected for RT-PCR analysis. A further reduction in type 1 interferon gene expression (IFIT1 (FIG. 8A), IFIT3 (FIG. 8B), MX1 (FIG. 8C), IRF7 (FIG. 8D), GBP2 (FIG. 8E) and LIGP1 (FIG. 8F)) was observed with the combination treatment compared to either monotherapy alone.

FIGS. 9A-9F show inhibition of lupus by mIL-2/CD25 administration in MRL/lpr mice. Male MRL/lpr mice. (12-14 weeks of age) with proteinuria level of 30 mg/dL were treated for 12 weeks with PBS or mIL-2/CD25 at 0.1, 0.2 or 0.4 mg/kg (s.c. 2×/week), or with Prednisolone (PO 3×/week) with n=10 per group. Treatment with mIL-2/CD25 resulted in a dose dependent reduction in the levels of proteinuria (FIG. 9A), the anti-dsDNA IgG titers (FIG. 9B), and the kidney histological scores (FIG. 9C), and dose dependent increases in the percentage of Tregs (CD4⁺CD25⁺Foxp3⁺) in blood (FIG. 9D) and the spleen (FIG. 9E), and increase in the CD25 MFI in Treg gate (CD4⁺CD25⁺Foxp3⁺) in the spleen (FIG. 9F). *p<0.05, **p<0.01, ***p<0.001, ****p<0.0001 vs. PBS group at week 12 by one-way ANOVA.

### DETAILED DESCRIPTION OF THE DISCLOSURE

Certain aspects of the present disclosure are directed to methods of treating a disease or disorder, an autoimmune disease and/or an inflammatory disease, e.g., systemic lupus erythematosus (SLE), in a subject in need thereof, comprising administering to the subject a dose of an Interleukin-2 (IL2) fusion protein, wherein the fusion protein comprises: (a) a first polypeptide comprising an IL2 polypeptide; and (b) a second polypeptide comprising an extracellular domain of an Interleukin-2 Receptor alpha (IL2Rα) polypeptide, wherein (i) the extracellular domain of the IL2Rα polypeptide has at least one fewer glycosylation compared to the extracellular domain of native IL2Rα (SEQ ID NO: 1); and/or (ii) the IL2 polypeptide has at least one fewer glycosylation compared to native IL2 (SEQ ID NO: 2). In some aspects, the dose is from about 0.1 mg to about 9 mg. In some aspects, the dose is greater than about 9 mg. In some aspects, the fusion protein is administered to the subject via an intravenous route, and the dose is from about 0.1 mg to about 9 mg. In some aspects, the fusion protein is administered to the subject via an intravenous route, and the dose is greater than about 9 mg. In some aspects, the fusion protein is administered to the subject via a subcutaneous route, and the dose is from about 1 mg to about 8 mg. In some aspects, the fusion protein is administered to the subject via a subcutaneous route, and the dose is greater than about 8 mg.

In some aspects, the method further comprises administering a corticosteroid, e.g., prednisolone, methylprednisolone, prednisone, hydrocortisone, dexamethasone, betamethasone, budesonide, triamcinolone, cortisone, desoxycorticosterone, fludrocortisone, or paramethasone.

In order that the present disclosure may be more readily understood, certain terms are first defined. As used in this application, except as otherwise expressly provided herein, each of the following terms shall have the meaning set forth below Additional definitions are set forth throughout the specification.

### I. Definitions

In order that the present disclosure can be more readily understood, certain terms are first defined. As used in this application, except as otherwise expressly provided herein, each of the following terms shall have the meaning set forth below. Additional definitions are set forth throughout the application.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description.

It is understood that wherever aspects are described herein with the language “comprising,” otherwise analogous aspects described in terms of “consisting of” and/or “consisting essentially of” are also provided.

The term “about” is used herein to mean approximately, roughly, around, or in the regions of. When the term “about” is used in conjunction with a numerical range, it modifies that range by extending the boundaries above and below the numerical values set forth. Thus, “about 10-20” means “about 10 to about 20.” In general, the term “about” can modify a numerical value above and below the stated value by a variance of, e.g., 10 percent, up or down (higher or lower).

As used herein, the term “recombinant” includes the expression from genes made by genetic engineering or otherwise by laboratory manipulation.

As used herein, “Interleukin-2”, “IL2”, or “IL-2” refers to any native or recombinant IL2 from any vertebrate source, including mammals such as primates (e.g., humans) and rodents (e.g., mice and rats), and domesticated or agricultural mammals unless otherwise indicated. The term encompasses unprocessed IL2, as well as, any form of IL2 that results from processing in the cell (i.e., the mature form of IL2). The term also encompasses naturally occurring variants and fragments of IL2, e.g. splice variants or allelic variants, and non-naturally occurring variants that have IL2 activity of the naturally occurring IL2.

Additional nucleic acid and amino acid sequences for IL2 are known. See, for example, GenBank Accession Nos: Q7JFM2 (Aotus lemurinus (Gray-bellied night monkey)); Q7JFM5 (Aotus nancymaae (Ma's night monkey)); P05016 (Bas taurus (Bovine)); Q29416 (Canisfamiliaris (Dog) (Canis lupus familiaris)); P36835 (Capra hircus (Goat)); and, P37997 (Equus caballus (Horse)).

Biologically active fragments and variants of IL2 retain IL2 activity. The phrase “biological activity of IL2” or “IL2 activity” refers to one or more of the biological activities of IL2, including but not limited to, the ability to stimulate IL2 receptor bearing lymphocytes. Such activity can be measured both in vitro and in vivo. IL2 is a global regulator of immune activity and the effects seen here are the sum of such activities. For example, it regulates survival activity (Bcl-2), induces T effector activity (IFN-gamma, Granzyme B, and Perforin), and/or promotes T regulatory activity (FoxP3).

Biologically active variants of IL2 are known. See, for example, US Application Publications 20060269515 and 20060160187 and WO 99/60128.

The term “secretory signal sequence” denotes a polynucleotide sequence that encodes a polypeptide (a “secretory peptide”) that, as a component of a larger polypeptide, directs the larger polypeptide through a secretory pathway of the cell in which it is synthesized. The larger polypeptide is commonly cleaved to remove the secretory peptide during the transit through the secretory pathway.

As used herein, a “mature” form of a fusion protein or polypeptide comprises the processed form of the polypeptide that has had the secretory peptide removed.

As used herein, the “unprocessed” form of the fusion protein retains the secretory peptide sequence.

The term “CD25,” “IL2 receptor a,” “IL2Rα,” or “IL2Rα” as used herein, refers to any native or recombinant IL2Rα from any vertebrate source, including mammals such as primates (e.g. humans) and rodents (e.g., mice and rats) and domesticated or agricultural mammals unless otherwise indicated. The term also encompasses naturally occurring variants of IL2Rα, e.g., splice variants or allelic variants, or non-naturally occurring variants that have IL2Rα activity. Human IL2 exerts its biological effects via signaling through its receptor system, IL2R. IL2 and its receptor (IL2R) are required for T-cell proliferation and other fundamental functions which are crucial for the immune response. IL2R consists of 3 non-covalently linked type I transmembrane proteins, which are the alpha (p55), beta (p75), and gamma (p65) chains. The human IL2R alpha chain contains an extracellular domain of 219 amino acids, a transmembrane domain of 19 amino acids, and an intracellular domain of 13 amino acids. The secreted extracellular domain of IL2R alpha (IL2R-α) can be employed in the fusion proteins describe herein.

Nucleic acid and amino acid sequences for IL2Rα are known. See, for example, GenBank Accession Nos: NP_001030597.1 (Pan troglodytes); NP_001028089.1 (Macaca mulatta); NM_001003211.1 (Canis lupus); NP_776783.1 (Bos taurus); NP_032393.3 (Mus musculus); and, NP_037295.1 (Rattus norvegicus).

Biologically active fragments and variants of the extracellular domain of IL2Rα are also provided. Such IL2Rα extracellular domain active variants or fragments will retain the IL2Rα extracellular domain activity. The phrase “biological activity of the IL2Rα extracellular domain” refers to one or more of the biological activities of extracellular domain of IL2Rα, including but not limited to, the ability to bind to IL2 and/or enhance intracellular signaling in IL2 receptor responsive cells. Non-limiting examples of biologically active fragments and variants of the IL2Rα are disclosed, for example, in Robb et al., Proc. Natl. Acad. Sci. USA, 85:5654-8 (1988). In some aspects, the biologically active fragments and variants of the IL2Rα disclosed herein comprise at least one fewer glycosylation compared to the extracellular domain of native IL2Rα.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring.

A “polypeptide” refers to a chain comprising at least two consecutively linked amino acid residues, with no upper limit on the length of the chain. One or more amino acid residues in the protein can contain a modification such as, but not limited to, glycosylation, phosphorylation or disulfide bond formation. A “protein” or “fusion protein” can comprise one or more polypeptides.

Also included in the present disclosure are fragments or variants of polypeptides, and any combination thereof. The term “fragment” or “variant” when referring to polypeptide binding domains or binding molecules of the present disclosure include any polypeptides which retain at least some of the properties (e.g., IL2 binding activity for IL2Rα) of the reference polypeptide. Fragments of polypeptides include proteolytic fragments, as well as deletion fragments, but do not include the naturally occurring full-length polypeptide (or mature polypeptide). Variants of polypeptide binding domains or binding molecules of the present disclosure include fragments as described above, and also polypeptides with altered amino acid sequences due to amino acid substitutions, deletions, or insertions. Variants can be naturally or non-naturally occurring. Non-naturally occurring variants can be produced using art-known mutagenesis techniques. Variant polypeptides can comprise conservative or non-conservative amino acid substitutions, deletions or additions.

As stated above, polypeptide variants include, e.g., modified polypeptides. Modifications include, e.g., acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation (Mei et al., Blood 116:270-79 (2010)), proteolytic processing, phosphorylation, prenylation, racemization, selenoylation, sulfation, transfer-RNA mediated addition of amino acids to proteins such as arginylation, and ubiquitination.

As used herein, the terms “amino acid corresponding to,” “site corresponding to,” or “equivalent amino acid” in a protein or nucleotide sequence is identified by alignment to maximize the identity or similarity between a first protein sequence, e.g., an IL2 sequence, and a second protein sequence, e.g., a second IL2 sequence. The number used to identify an equivalent amino acid in a second protein sequence is based on the number used to identify the corresponding amino acid in the first protein sequence. In some aspects, the term “corresponding to” refers to the relationship of a mutation at one or more amino acids in a polypeptide or one or more nucleotides in a polynucleotide. By way of a non-limiting example, a specific amino acid (e.g., S50) of a polynucleotide (e.g., SEQ ID NO: 1) as disclosed herein refers to the 50^{th} amino acid—a serine—in SEQ ID NO: 1.

As used herein the term “associated with” refers to a covalent or non-covalent bond formed between a first amino acid chain and a second amino acid chain. In one aspect, the term “associated with” means a covalent, non-peptide bond or a non-covalent bond. This association can be indicated by a colon, i.e., (:). In another aspect, it means a covalent bond except a peptide bond. For example, the amino acid cysteine comprises a thiol group that can form a disulfide bond or bridge with a thiol group on a second cysteine residue. In most naturally occurring IgG molecules, the CH1 and CL regions are associated by a disulfide bond and the two heavy chains are associated by two disulfide bonds at positions corresponding to 239 and 242 using the Kabat numbering system (position 226 or 229, EU numbering system). Examples of covalent bonds include, but are not limited to, a peptide bond, a metal bond, a hydrogen bond, a disulfide bond, a sigma bond, a pi bond, a delta bond, a glycosidic bond, an agnostic bond, a bent bond, a dipolar bond, a Pi backbond, a double bond, a triple bond, a quadruple bond, a quintuple bond, a sextuple bond, conjugation, hyperconjugation, aromaticity, hapticity, or antibonding. Non-limiting examples of non-covalent bond include an ionic bond (e.g., cation-pi bond or salt bond), a metal bond, an hydrogen bond (e.g., dihydrogen bond, dihydrogen complex, low-barrier hydrogen bond, or symmetric hydrogen bond), van der Walls force, London dispersion force, a mechanical bond, a halogen bond, aurophilicity, intercalation, stacking, entropic force, or chemical polarity.

The term “comparable” as used herein means a compared rate or level resulted from using, e.g., the fusion protein is equal to, substantially equal to, or similar to the reference rate or level. The term “similar” as used herein means a compared rate or level has a difference of no more than 10% or no more than 15% from the reference rate or level. The term “substantially equal” means a compared rate or level has a difference of no more than 0.01%, 0.5% or 1% from the reference rate or level.

The term “expression” as used herein refers to a process by which a polynucleotide produces a gene product, for example, an RNA or a polypeptide.

A “fusion” or “fusion protein” comprises a first amino acid sequence linked in frame to a second amino acid sequence with which it is not naturally linked in nature. The amino acid sequences which normally exist in separate proteins can be brought together in the fusion polypeptide, or the amino acid sequences which normally exist in the same protein can be placed in a new arrangement in the fusion polypeptide, e.g., fusion of an IL2 protein with an IL2-Ra protein. A fusion protein is created, for example, by chemical synthesis, or by creating and translating a polynucleotide in which the peptide regions are encoded in the desired relationship. A fusion protein can further comprise a second amino acid sequence associated with the first amino acid sequence by a covalent, non-peptide bond or a non-covalent bond. Upon transcription/translation, a single protein is made. In this way, multiple proteins, or fragments thereof can be incorporated into a single polypeptide. “Operably linked” is intended to mean a functional linkage between two or more elements. For example, an operable linkage between two polypeptides fuses both polypeptides together in frame to produce a single polypeptide fusion protein. In one aspect, the fusion protein further comprises a third polypeptide which, as discussed in further detail below, can comprise a linker sequence.

As used herein, the term “BMS-986326” or “BMS-986326-01” refers to a recombinant fusion protein of human interleukin-2 (IL2) and the extracellular domain portion of the alpha subunit of the human IL2 receptor (CD25), which forms a non-covalent, self-blocking, homodimer structure having a mass of approximately 83 kilodaltons (kDa). The IL2 and CD25 moieties are linked to one another by the small peptide linker sequence of (GGGS)₃. BMS-986326 is optimized for prolonged pharmacokinetics (PK) and regulatory T cell (Treg) selectivity and provides low-dose IL2 receptor (IL2R) agonism. As an inactive homodimer, BMS-986326 sterically inhibits IL2R binding until monomer release, providing a mechanism for avoiding target-mediated drug disposition (TMDD) and renal clearance and augmenting Treg-selective in vivo activity through slow release of active monomer. Monomer release enables the molecule to engage with IL2R to initiate signaling, with greater potency and selectivity for cells expressing high levels of CD25, such as Tregs. Toxicology studies in rats and monkeys have demonstrated an acceptable tolerability profile for BMS-986326. In some aspects, BMS-986326 comprises the amino acid sequence as set forth in SEQ ID NO: 5, which corresponds to SEQ ID NO: 16 in U.S. Publ. No. 2009-0359672. U.S. Publ. No. 2009-0359672 is herein incorporated by reference in its entirety.

An “Fc region” (fragment crystallizable region), “Fc domain,” or “Fc” refers to the C-terminal region of the heavy chain of an antibody that mediates the binding of the immunoglobulin to host tissues or factors, including binding to Fc receptors located on various cells of the immune system (e.g., effector cells) or to the first component (C1q) of the classical complement system. Thus, an Fc region comprises the constant region of an antibody excluding the first constant region immunoglobulin domain (e.g., CH1 or CL). In IgG, IgA and IgD antibody isotypes, the Fc region comprises two identical protein fragments, derived from the second (CH2) and third (CH3) constant domains of the antibody's two heavy chains; IgM and IgE Fc regions comprise three heavy chain constant domains (CH domains 2-4) in each polypeptide chain. The IgG isotype is divided in subclasses in certain species: IgG1, IgG2, IgG3 and IgG4 in humans, and IgG1, IgG2a, IgG2b and IgG3 in mice. For IgG, the Fc region comprises immunoglobulin domains CH2 and CH3 and the hinge between CH1 and CH2 domains. Although the definition of the boundaries of the Fc region of an immunoglobulin heavy chain might vary, as defined herein, the human IgG heavy chain Fc region is defined to stretch from an amino acid residue D221 for IgG1, V222 for IgG2, L221 for IgG3 and P224 for IgG4 to the carboxy-terminus of the heavy chain, wherein the numbering is according to the EU index as in Kabat. The CH2 domain of a human IgG Fc region extends from amino acid 237 to amino acid 340, and the CH3 domain is positioned on C-terminal side of a CH2 domain in an Fc region, i.e., it extends from amino acid 341 to amino acid 447 or 446 (if the C-terminal lysine residue is absent) or 445 (if the C-terminal glycine and lysine residues are absent) of an IgG. As used herein, the Fc region can be a native sequence Fc, including any allotypic variant, or a variant Fc (e.g., a non-naturally-occurring Fc).

An “Fc receptor” or “FcR” is a receptor that binds to the Fc region of an immunoglobulin. FcRs that bind to an IgG antibody comprise receptors of the FcγR family, including allelic variants and alternatively spliced forms of these receptors. The FcγR family consists of three activating (FcγRI, FcγRIII, and FcγRIV in mice; FcγRIA, FcγRIIA, and FcγRIIIA in humans) and one inhibitory (FcγRIIB) receptor. Various properties of human FcγRs are known in the art. The majority of innate effector cell types co-express one or more activating FcγR and the inhibitory FcγRIIB, whereas natural killer (NK) cells selectively express one activating Fc receptor (FcγRIII in mice and FcγRIIIA in humans) but not the inhibitory FcγRIIB in mice and humans. Human IgG1 binds to most human Fc receptors and is considered equivalent to murine IgG2a with respect to the types of activating Fc receptors that it binds to.

The terms “inserted,” “is inserted,” “inserted into,” or grammatically related terms, as used herein refers to the position of a heterologous moiety (e.g., a half-life extending moiety) in a fusion polypeptide relative to the analogous position in specified protein. As used herein the terms refer to the characteristics of the recombinant polypeptide disclosed herein, and do not indicate, imply or infer any methods or process by which the fusion polypeptide was made.

“Heterologous” and “heterologous moiety” in reference to a polypeptide or polynucleotide is a polypeptide or polynucleotide that originates from a different protein or polynucleotide. The additional components of the fusion protein can originate from the same organism as the other polypeptide components of the fusion protein, or the additional components can be from a different organism than the other polypeptide components of the fusion protein. For instance, a heterologous polypeptide can be synthetic, or derived from a different species, different cell type of an individual, or the same or different type of cell of distinct individuals. In one aspect, a heterologous moiety is a polypeptide fused to another polypeptide to produce a fusion polypeptide or protein. In another aspect, a heterologous moiety is a non-polypeptide such as PEG conjugated to a polypeptide or protein. Non-limiting examples of heterologous moieties disclosed herein are glycine/serine linkers (e.g., GGGSGGGSGGGS (SEQ ID NO: 6) (also noted as (Gly₃Ser)₃)).

A “native sequence Fc region” or “native sequence Fc” comprises an amino acid sequence that is identical to the amino acid sequence of an Fc region found in nature. Native sequence human Fc regions include a native sequence human IgG1 Fc region; native sequence human IgG2 Fc region; native sequence human IgG3 Fc region; and native sequence human IgG4 Fc region as well as naturally-occurring variants thereof. Native sequence Fc include the various allotypes of Fcs (see, e.g., Jefferis et al. (2009) mAbs 1: 1).

The term “EC₅₀” in the context of an in vitro or in vivo assay using fusion protein refers to the concentration of a fusion protein that induces a response that is 50% of the maximal response, i.e., halfway between the maximal response and the baseline.

“Conservative amino acid substitutions” refer to substitutions of an amino acid residue with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). In some aspects, a predicted nonessential amino acid residue in the IL2 fusion protein is replaced with another amino acid residue from the same side chain family. Methods of identifying nucleotide and amino acid conservative substitutions which do not eliminate antigen binding are well-known in the art (see, e.g., Brummell et al., Biochem. 32: 1180-7 (1993); Kobayashi et al. Protein Eng. 12(10):879-84 (1999); and Burks et al. Proc. Natl. Acad. Sci. USA 94:412-17 (1997)).

A polynucleotide, which encodes a gene product, e.g., a polypeptide, can include a promoter and/or other transcription or translation control elements operably associated with one or more coding regions. Other transcription control elements, besides a promoter, for example enhancers, operators, repressors, and transcription termination signals, can also be operably associated with a coding region to direct gene product expression.

The term “percent sequence identity,” “percent identity,” “sequence identity,” or “identity” are used interchangeably and refers to the number of identical matched positions shared between two polynucleotide or polypeptide sequences over a comparison window, taking into account additions or deletions (i.e., gaps) that must be introduced for optimal alignment of the two sequences. A matched position is any position where an identical nucleotide or amino acid is presented in both the target and reference sequence. Gaps presented in the target sequence are not counted since gaps are not nucleotides or amino acids. Likewise, gaps presented in the reference sequence are not counted since target sequence nucleotides or amino acids are counted, not nucleotides or amino acids from the reference sequence.

The comparison of sequences and determination of percent identity between two sequences can be accomplished using a mathematical algorithm, as described in the non-limiting examples below.

The percentage of sequence identity is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity. The comparison of sequences and determination of percent sequence identity between two sequences may be accomplished using readily available software both for online use and for download. Suitable software programs are available from various sources, and for alignment of both protein and nucleotide sequences. One suitable program to determine percent sequence identity is bl2seq, part of the BLAST suite of programs available from the U.S. government's National Center for Biotechnology Information BLAST web site (blast.ncbi.nlm.nih.gov). Bl2seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

The percent identity between two nucleotide sequences can be determined using the GAP program in the GCG software package (available at worldwideweb.gcg.com), using a NWSgapdna. CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (CABIOS, 4: 11-17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48):444-453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

The nucleic acid and protein sequences described herein can further be used as a “query sequence” to perform a search against public databases to, for example, identify related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, word length=12 to obtain nucleotide sequences homologous to the nucleic acid molecules described herein. BLAST protein searches can be performed with the XBLAST program, score=50, word length=3 to obtain amino acid sequences homologous to the protein molecules described herein. To obtain gapped alignments for comparison purposes, Gapped BLAST can be utilized as described in Altschul et al., Nucleic Acids Res. 25(17):3389-402 (1997). When utilizing BLAST and Gapped BLAST programs, the default parameters of the respective programs (e.g., XBLAST and NBLAST) can be used. See worldwideweb.ncbi.nlm.nih.gov.

As used herein, “administering” refers to the physical introduction of a composition comprising a therapeutic agent to a subject, using any of the various methods and delivery systems known to those skilled in the art. Different routes of administration for the IL2 fusion protein described herein include intravenous, intraperitoneal, intramuscular, subcutaneous, spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural and intrasternal injection and infusion, as well as in vivo electroporation. Alternatively, an antibody described herein can be administered via a non-parenteral route, such as a topical, epidermal or mucosal route of administration, for example, intranasally, orally, vaginally, rectally, sublingually or topically. Administering can also be performed, for example, once, a plurality of times, and/or over one or more extended periods.

“Dosing interval,” as used herein, means the amount of time that elapses between multiple doses (two or more doses) being administered to a subject. The comparison of dosing interval can be carried out in a single subject or in a population of subjects and then the average obtained in the population can be calculated. The dosing interval can be the amount of time between a dose given by one route (intravenous) and a dose given by another route (subcutaneous). The dosing interval as used herein refers to two doses that are adjacent in time to each other.

An “immune response” is as understood in the art, and generally refers to a biological response within a vertebrate against foreign agents or abnormal cells, which response protects the organism against these agents and diseases caused by them. An immune response is mediated by the action of one or more cells of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens or abnormal cells, or, in cases of autoimmunity or pathological inflammation, normal human cells or tissues. An immune reaction includes, e.g., activation or inhibition of a T cell, e.g., an effector T cell, a Th cell, a CD4⁺ cell, a CD8⁺ T cell, or a Treg cell, or activation or inhibition of any other cell of the immune system, e.g., NK cell.

An “immunomodulator” or “immunoregulator” refers to an agent, e.g., an agent targeting a component of a signaling pathway that can be involved in modulating, regulating, or modifying an immune response. “Modulating,” “regulating,” or “modifying” an immune response refers to any alteration in a cell of the immune system or in the activity of such cell (e.g., an effector T cell, such as a Th1 cell). More particularly, as used herein, the term “modulating” includes inducing, inhibiting, potentiating, elevating, increasing, or decreasing a given activity or response. Such modulation includes stimulation or suppression of the immune system which can be manifested by an increase or decrease in the number of various cell types, an increase or decrease in the activity of these cells, or any other changes which can occur within the immune system. Both inhibitory and stimulatory immunomodulators have been identified. In some aspects, the immunomodulator targets a molecule on the surface of a T cell. An “immunomodulatory target” or “immunoregulatory target” is a molecule, e.g., a cell surface molecule, that is targeted for binding by, and whose activity is altered by the binding of, a substance, agent, moiety, compound or molecule. Immunomodulatory targets include, for example, receptors on the surface of a cell (“immunomodulatory receptors”) and receptor ligands (“immunomodulatory ligands”).

“Immunotherapy” refers to the treatment of a subject afflicted with, or at risk of contracting or suffering a recurrence of, a disease by a method comprising inducing, enhancing, suppressing or otherwise modifying the immune system or an immune response.

“Immuno stimulating therapy” or “immuno stimulatory therapy” refers to a therapy that results in increasing (inducing or enhancing) an immune response in a subject.

“Potentiating an endogenous immune response” means increasing the effectiveness or potency of an existing immune response in a subject. This increase in effectiveness and potency can be achieved, for example, by overcoming mechanisms that suppress the endogenous host immune response or by stimulating mechanisms that enhance the endogenous host immune response.

“T effector” (“Teff”) cells refers to T cells (e.g., CD4⁺ and CD8⁺ T cells) with cytolytic activities as well as T helper (Th) cells, e.g., Th1 cells, which cells secrete cytokines and activate and direct other immune cells, but does not include regulatory T cells (Treg cells). Certain IL2 fusion proteins described herein activate Teff cells, e.g., CD4⁺ and CD8⁺ Teff cells and Th1 cells.

An increased ability to stimulate an immune response or the immune system, can result from an enhanced agonist activity of T cell co-stimulatory receptors and/or an enhanced antagonist activity of inhibitory receptors. An increased ability to stimulate an immune response or the immune system can be reflected by a fold increase of the EC₅₀ or maximal level of activity in an assay that measures an immune response, e.g., an assay that measures changes in cytokine or chemokine release, cytolytic activity (determined directly on target cells or indirectly via detecting CD107a or granzymes) and proliferation. The ability to stimulate an immune response or the immune system activity can be enhanced by at least 10%, 30%, 50%, 75%, 2 fold, 3 fold, 5 fold or more.

The terms “linked” and “fused” as used herein refers to a first amino acid sequence or nucleotide sequence covalently or non-covalently joined to a second amino acid sequence or nucleotide sequence, respectively. The first amino acid or nucleotide sequence can be directly joined or juxtaposed to the second amino acid or nucleotide sequence or alternatively an intervening sequence can covalently join the first sequence to the second sequence. The term “linked” means not only a fusion of a first amino acid sequence to a second amino acid sequence at the C-terminus or the N-terminus, but also includes insertion of the whole first amino acid sequence (or the second amino acid sequence) into any two amino acids in the second amino acid sequence (or the first amino acid sequence, respectively). In one aspect, the first amino acid sequence is linked to a second amino acid sequence by a peptide bond or a linker. The first nucleotide sequence can be linked to a second nucleotide sequence by a phosphodiester bond or a linker. The linker can be a peptide or a polypeptide (for polypeptide chains) or a nucleotide or a nucleotide chain (for nucleotide chains) or any chemical moiety (for both polypeptide and polynucleotide chains). The term “linked” is also indicated by a hyphen (-).

As used herein, the term “T cell-mediated response” refers to a response mediated by T cells, including effector T cells (e.g., CD8⁺ cells) and helper T cells (e.g., CD4⁺ cells). T cell mediated responses include, for example, T cell cytotoxicity and proliferation.

As used herein, the term “cytotoxic T lymphocyte (CTL) response” refers to an immune response induced by cytotoxic T cells. CTL responses are mediated primarily by CD8⁺ T cells.

As used herein, the terms “inhibits” or “blocks” (e.g., referring to inhibition/blocking of binding of IL2 to IL2Rα on cells) are used interchangeably and encompass both partial and complete inhibition/blocking. In some aspects, the IL2 fusion protein inhibits binding of IL2 to IL2Rα by at least about 50%, for example, about 60%, 70%, 80%, 90%, 95%, 99%, or 100%, determined, e.g., as further described herein. In some aspects, the IL2 fusion protein inhibits binding of IL2 to IL2Rα by no more than 50%, for example, by about 40%, 30%, 20%, 10%, 5% or 1%, determined, e.g., as further described herein.

The terms “treat,” “treating,” and “treatment,” as used herein, refer to any type of intervention or process performed on, or administering an active agent to, the subject with the objective of reversing, alleviating, ameliorating, inhibiting, or slowing down or preventing the progression, development, severity or recurrence of a symptom, complication, condition or biochemical indicia associated with a disease or enhancing overall survival. Treatment can be of a subject having a disease or a subject who does not have a disease (e.g., for prophylaxis). When provided prophylactically, the fusion protein disclosed herein is provided in advance of any symptom. The prophylactic administration of the substance serves to prevent or attenuate any subsequent symptom.

By “enhancing the efficacy” or “enhancing the immunogenicity” with regard to a fusion protein, pharmaceutical composition, or vaccine is intended improving an outcome, for example, as measured by a change in a specific value, such as an increase or a decrease in a particular parameter of an activity of a fusion protein, pharmaceutical composition, or vaccine associated with protective immunity. In one aspect, enhancement refers to at least a 5%, 10%, 25%, 50%, 100% or greater than 100% increase in a particular parameter. In another aspect, enhancement refers to at least a 5%, 10%, 25%, 50%, 100% or greater than 100% decrease in a particular parameter. In one example, enhancement of the efficacy/immunogenicity of a vaccine refers to an increase in the ability of the vaccine to inhibit or treat disease progression, such as at least a 5%, 10%, 25%, 50%, 100%, or greater than 100% increase in the effectiveness of the vaccine for that purpose.

Similarly, by “overcoming a suppressed immune response” with regard to a fusion protein, pharmaceutical composition, or vaccine is intended improving an outcome, for example, as measured by a change in a specific value, such as a return to a formerly positive value in a particular parameter of an activity of a vaccine associated with protective immunity. In one aspect, overcoming refers to at least a 5%. 10%, 25%, 50%, 100% or greater than 100% increase in a particular parameter. In one example, overcoming a suppressed immune response to a fusion protein, pharmaceutical composition, or vaccine refers to a renewed ability of the fusion protein, pharmaceutical composition, or vaccine to inhibit or treat disease progression, such as at least a 5%, 10%, 25%, 50%, 100%, or greater than 100% renewal in the effectiveness of the vaccine for that purpose.

A “therapeutic dose,” “dose,” or “dosing amount” as used (interchangeably) herein, means a dose that achieves a therapeutic goal, as described herein. In some aspects, a “therapeutic dose” means a dose that induces an immune tolerance in a subject. In certain aspects, a “therapeutic dose” means a dose that induces an immune tolerance in a subject within a specified time to tolerance period, e.g., within 12 weeks of administration of the first dose. A “dose” of an IL2 fusion protein refers to the amount of the IL2 fusion protein sufficient to elicit a desired biological response. As will be appreciated by one of ordinary skill in the art, the absolute amount of a particular IL2 fusion protein that is effective can vary depending on such factors as the desired biological endpoint, the IL2 fusion protein to be delivered, the target cell or tissue, and the like. One of ordinary skill in the art will further understand that an effective amount can be administered in a single dose, or can be achieved by administration of multiple doses (i.e., 1, 2, 3, 4, 5, 6, 7, 8, 9, 10 or more doses). The ability of a therapeutic agent to promote disease regression or inhibit the development or recurrence of the disease can be evaluated using a variety of methods known to the skilled practitioner, such as in human subjects during clinical trials, in animal model systems predictive of efficacy in humans, or by assaying the activity of the agent in in vitro assays.

“Treat,” “treatment,” or “treating,” as used herein refers to, e.g., the reduction in severity of a disease or condition; the reduction in the duration of a condition course; the amelioration or elimination of one or more symptoms associated with a disease or condition; the provision of beneficial effects to a subject with a disease or condition, without necessarily curing the disease or condition.

“Pharmaceutical formulation” or “pharmaceutical composition,” as used herein, refers to a preparation which is in such form as to permit the biological activity of the active ingredient to be effective, and which contains no additional components which are unacceptably toxic to a subject to which the pharmaceutical formulation or composition would be administered. The pharmaceutical formulation or composition can be sterile. In some aspects, the pharmaceutical formulation or composition is suitable for therapeutic use in a human subject.

The term “patient” includes human and other mammalian subjects that receive either prophylactic or therapeutic treatment.

As used herein, the term “subject” includes any human or non-human animal. For example, the methods and compositions described herein can be used to treat a subject having an immune disease. The term “non-human animal” includes all vertebrates, e.g., mammals and non-mammals, such as non-human primates, sheep, dog, cow, chickens, amphibians, reptiles, etc.

The term “weight based” dose or dosing as referred to herein means that a dose that is administered to a patient is calculated based on the weight of the patient. For example, when a patient with 60 kg body weight requires 3 mg/kg of an anti-IL2 antibody, one can calculate and use the appropriate amount of the IL2 fusion protein (i.e., 180 mg) for administration.

The use of the term “flat dose” with regard to the methods and dosages described herein means a dose that is administered to a patient without regard for the weight or body surface area (BSA) of the patient. The flat dose is therefore not provided as a mg/kg dose, but rather as an absolute amount of the agent (e.g., the IL2 fusion protein). For example, a 60 kg person and a 100 kg person would receive the same dose of an antibody (e.g., 480 mg of an IL2 fusion protein).

As used herein, the terms “ug” and “uM“are used interchangeably with” g” and “M,” respectively.

As used herein, the term “Investigational Product” or “IP” includes BMS-986326 as well as placebo (0.9% sodium chloride). In some instances, Investigational Products may be administered to a subject by any means known in the art, such as, for example, intravenously or subcutaneously.

Any compositions or methods provided herein can be combined with one or more of any of the other compositions and methods provided herein.

References made to amino acid numbering of immunoglobulins or immunoglobulin fragments, or regions, are all based on Kabat et al. 1991, Sequences of Proteins of Immunological Interest, U. S. Department of Public Health, Bethesda; MD. (The FcRn receptor has been isolated from several mammalian species including humans. The sequences of the human FcRn, rat FcRn, and mouse FcRn are known (Story et al., J. Exp. Med. 180: 2377 (1994).) An Fc can comprise the CH2 and CH3 domains of an immunoglobulin with or without the hinge region of the immunoglobulin. Exemplary Fc variants are provided in WO 2004/101740 and WO 2006/074199.

Units, prefixes, and symbols are denoted in their Systéme International de Unites (SI) accepted form. Numeric ranges are inclusive of the numbers defining the range. Unless otherwise indicated, amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Various aspects described herein are described in further detail in the following subsections.

### II. Methods of Treating a Disease or Disorder in a Subject

The disclosure provides methods of treating a disease or disorder in a subject in need thereof, comprising administering to the subject one or more doses of an Interleukin-2 (IL2) fusion protein, wherein the fusion protein comprises: (a) a first polypeptide comprising an IL2 polypeptide, and (b) a second polypeptide comprising an extracellular domain of an Interleukin-2 Receptor alpha (IL2Rα) polypeptide; wherein (i) the extracellular domain of the IL2Rα polypeptide has at least one fewer glycosylation compared to the extracellular domain of native IL2Rα (SEQ ID NO: 1); and/or (ii) the IL2 polypeptide has at least one fewer glycosylation compared to native IL2 (SEQ ID NO: 2). In some aspects, the present method further comprises administering to the subject a corticosteroid. In some aspects, the corticosteroid is prednisolone, methylprednisolone, prednisone, hydrocortisone, dexamethasone, betamethasone, budesonide, triamcinolone, cortisone, desoxycorticosterone, fludrocortisone, or paramethasone.

The importance of the IL-2 signaling pathway on Tregs has been demonstrated by the appearance of systemic autoimmunity in mice or humans lacking components of the IL-2 signaling pathway. Dysregulation of Treg cell numbers and/or function has been implicated in numerous immune-mediated conditions. See, for example, Bluestone, J. A., et al., J Clin Invest. 125:2250-60 (2015); and Dominguez-Villar, M and Hafler, D. A., Nat Immunol. 19:665-73 (2018). Autoimmune risk variants in the IL-2, IL-2Rα, and IL-2Rβ loci have been identified through genome-wide association studies (GWAS) and associated with immune-mediated diseases including inflammatory bowel disease (IBD), Type-1 autoimmune diabetes (T1DM), multiple sclerosis (MS), and rheumatoid arthritis (RA). See, for example, Abbas, A. K., et al., Sci Immunol. 3, eaat1482 (2018). Mutations affecting the key Treg lineage transcription factor FoxP3 cause the autoimmune lymphoproliferative disease Immune Dysregulation, Polyendocrinopathy, enteropathy, X-linked (IPEX) syndrome, resulting from loss of functional Treg. In addition, patients with CD25 deficiency, which results from mutations in IL-2RA, suffer from immune dysregulation similar to IPEX syndrome. See, for example, Verbsky, J. W. and Chatila, T., Curr Opin Pediatr. 25(6):708-14 (2013). Genetic data are consistent with a central role for IL-2 in Treg function and suppression of autoimmunity in both mice and humans.

It is generally assumed that the primary mechanism by which IL-2 deficiency contributes to the loss of tolerance and immunopathology in SLE patients is by disrupting Treg homeostasis. See, for example, Klatzmann, D., and Abbas, A. K., Nat Rev Immunol. 15:283-94 (2015); and Ballesteros-Tato, A. and Papillion, A., Curr Opin Immunol. 61:39-45 (2019). While some studies have suggested that there is a relative increase in the frequency of Tregs in patients with active SLE disease, these Tregs have been reported to have lower CD25 expression, suggesting functional impairment. See, for example, Von Spee-Mayer, C., et al., Ann Rheum Dis. 75:1407-15 (2016). Although the functional impairment of Tregs in SLE patients is not clearly understood, three independent clinical studies in SLE have demonstrated increased Tregs and decreased disease activity and increased Tregs after low-dose IL-2 treatment. See, for example, He, J., et al., Arthritis Rheumatol. 67(suppl 10) (2015); Humrich, J. Y., et al. Ann Rheum Dis. 74:791-92 (2015); and Von Spee-Mayer, C., et al., Ann Rheum Dis. 75:1407-15 (2016). The association between low-dose IL-2 administration, Treg expansion, and reduced immunopathology has been shown in other forms of immune-mediated disorders, including type I diabetes (see, for example, Dwyer, C. J., et al., Curr Diab Rep. 16:46 (2016)), HCV-induced vasculitis (see, for example, Dupont, G., et al., Cytokine. 69:146-9 (2014)), alopecia areata (see, for example, Castela, E., et al., JAMA Dermatol. 150:748-51 (2014)), and GvHD (see, for example, Koreth, J., et al., N Engl J Med. 365:2055-66 (2011)). These studies indicate a causal correlation between the IL-2-dependent expansion of Tregs and the clinical benefits observed following low-dose IL-2 treatment.

Systemic lupus erythematosus (SLE) has been described as an IL2-deficient state, and IL-2 deficiency is associated with SLE progression. See, for example, Mizui, M. and Tsokos, G. C., Front Immunol. 9: Article 786 (2018). Cultured peripheral blood mononuclear cells and CD4+ T cells from patients with SLE demonstrate deficient ex vivo IL-2 production. See, for example, Comte, D., et al., Arthritis & Rheumatology 69:808-13 (2017).

The present disclosure therefore provides safe and efficacious dosages for the IL2 fusion protein disclosed herein for treatment of a disease or condition, e.g, an autoimmune disease and/or inflammatory disease, e.g., SLE.

### II. A. Doses and Routes of Administration

In some aspects, the dose of the IL2 fusion protein is from about 0.1 mg to about 9 mg. In other aspects, the dose of the IL2 fusion protein is greater than about 9 mg.

The dose of the IL2 fusion protein may be administered to the subject by a topical, epidermal, mucosal, intranasal, oral, vaginal, rectal, sublingual, topical, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural or intrasternal route.

In some aspects, the fusion protein is administered to the subject via an intravenous route. In some aspects, the AUC[0-336 hours (h)] of the dose administered i.v. is controlled such that the mean exposure (AUC[INF]) is limited. In some aspects, the AUC[0-336 hours (h)] of the dose is lower than about 757 μg·h/ml.

In some aspects, the AUC[0-336 hours (h)] of the dose is lower than about 750 μg·h/ml, about 740 μg·h/ml, about 730 μg·h/ml, about 720 μg·h/ml, about 710 μg·h/ml, about 700 μg·h/ml, about 690 μg·h/ml, about 680 μg·h/ml, about 670 μg·h/ml, about 660 μg·h/ml, about 650 μg·h/ml, about 640 μg·h/ml, about 630 μg·h/ml, about 620 μg·h/ml, about 610 μg·h/ml, about 600 μg·h/ml, about 590 μg·h/ml, about 580 μg·h/ml, about 570 μg·h/ml, about 560 μg·h/ml, or about 550 μg·h/ml.

In some aspects, the fusion protein is administered via an intravenous route at a dose of between about 0.3 mg to about 9 mg. In some aspects, the fusion protein is administered to the subject via an intravenous route at a dose of between about 1 mg and about 9 mg, between about 2 mg and about 9 mg, between about 3 mg and about 9 mg, between about 4 mg and about 9 mg, between about 5 mg and about 9 mg, between about 6 mg and about 9 mg, between about 7 mg and about 9 mg, between about 8 mg and about 9 mg, between about 1 mg and about 8 mg, between about 2 mg and about 8 mg, between about 3 mg and about 8 mg, between about 4 mg and about 8 mg, between about 5 mg and about 8 mg, between about 6 mg and about 8 mg, between about 7 mg and about 8 mg, between about 1 mg and about 7 mg, between about 2 mg and about 7 mg, between about 3 mg and about 7 mg, between about 4 mg and about 7 mg, between about 5 mg and about 7 mg, between about 6 mg and about 7 mg. In some aspects, the dose administered via an intravenous route is between about 3 mg and about 9 mg. In some aspects, the dose administered via an intravenous route is between about 6 mg and about 9 mg.

In some aspects, the fusion protein is administered to the subject via an intravenous route at a dose of between about 0.1 mg and about 6 mg, between about 1 mg and about 6 mg, between about 2 mg and about 6 mg, between about 3 mg and about 6 mg, between about 4 mg and about 6 mg, or between about 5 mg and about 6 mg, between about 1 mg and about 5 mg, between about 2 mg and about 5 mg, between about 3 mg and about 5 mg, between about 4 mg and about 5 mg, between about 1 mg and about 4 mg, between about 2 mg and about 4 mg, between about 3 mg and about 4 mg, between about 1 mg and about 3 mg, or between about 2 mg and about 3 mg. In some aspects, the dose administered via an intravenous route is between about 0.1 mg and about 3 mg. In some aspects, the dose administered via an intravenous route is between about 0.1 mg and about 1 mg. In some aspects, the dose administered via an intravenous route is between about 0.1 mg and about 0.3 mg. In some aspects, the dose administered via an intravenous route is between about 0.3 mg and about 6 mg. In some aspects, the dose administered via an intravenous route is between about 1 mg and about 3 mg.

In some aspects, the dose administered via an intravenous route is about 0.1 mg, about 0.3 mg, about 1 mg, about 2 mg, about 3 mg, about 4 mg, about 5 mg, about 6 mg, about 7 mg, about 8 mg, or about 9 mg. In some aspects, the dose administered via an intravenous route is greater than about 9 mg.

In some aspects, the fusion protein is administered to the subject via a subcutaneous route. In some aspects, the AUC[0-336 hours (h)] of the dose administered s.c. is controlled such that the mean exposure (AUC[INF]) is limited. In some aspects, the AUC(0-504 h) of the dose is lower than about 306 μg·h/ml.

In some aspects, the AUC(0-504 h) of the dose is lower than about 300 μg·h/ml, about 290 μg·h/ml, about 280 μg·h/ml, about 270 μg·h/ml, about 260 μg·h/ml, about 250 μg·h/ml, about 240 μg·h/ml, about 230 μg·h/ml, about 220 μg·h/ml, about 210 μg·h/ml, about 200 μg·h/ml, about 190 μg·h/ml, about 180 μg·h/ml, about 170 μg·h/ml, about 160 μg·h/ml, or about 150 μg·h/ml.

In some aspects, the fusion protein is administered to the subject via a subcutaneous route at a dose of between about 1 mg and about 8 mg, between about 2 mg and about 8 mg, between about 3 mg and about 8 mg, between about 4 mg and about 8 mg, between about 5 mg and about 8 mg, between about 6 mg and about 8 mg, between about 7 mg and about 8 mg, between about 1 mg and about 7 mg, between about 2 mg and about 7 mg, between about 3 mg and about 7 mg, between about 4 mg and about 7 mg, between about 5 mg and about 7 mg, between about 6 mg and about 7 mg, between about 1 mg and about 6 mg, between about 2 mg and about 6 mg, between about 3 mg and about 6 mg, between about 4 mg and about 6 mg, or between about 5 mg and about 6 mg, between about 1 mg and about 5 mg, between about 2 mg and about 5 mg, between about 3 mg and about 5 mg, between about 4 mg and about 5 mg, between about 1 mg and about 4 mg, between about 2 mg and about 4 mg, between about 3 mg and about 4 mg, between about 1 mg and about 3 mg, or between about 2 mg and about 3 mg. In some aspects, the dose administered via a subcutaneous route is between about 3 mg and about 8 mg. In some aspects, the dose administered via a subcutaneous route is between about 6 mg and about 8 mg. In some aspects, the dose administered via a subcutaneous route is between about 1 mg to about 6 mg. In some aspects, the dose administered via a subcutaneous route is between about 1 mg to about 3 mg. In some aspects, the dose administered via a subcutaneous route is between about 3 mg to about 6 mg.

In some aspects, the dose administered via subcutaneous route is about 1 mg, about 3 mg, about 6 mg, or about 8 mg. In some aspects, the dose administered via subcutaneous route is greater than about 8 mg.

In some aspects, the present method includes administering multiple doses (i.e., two or more doses) to a subject in need thereof at a dosing interval between two doses. In some aspects, the dosing interval (e.g., subcutaneous or intravenous) is at least about one day, at least about two days, at least about three days, at least about four days, at least about five days, or at least about six days. In some aspects, the dosing interval (e.g., subcutaneous or intravenous) is at least about one week, at least about two weeks, at least about three weeks, at least about four weeks, at least about a month, at least about five weeks, at least about six weeks, at least about seven weeks, at least about eight weeks, at least about two months, at least about nine weeks, at least about ten weeks, at least about eleven weeks, at least about twelve weeks, or at least about three months. In some aspects, the dosing interval is at least about three weeks. In some aspects, the dosing interval is at least about two weeks. In some aspects, the dosing interval is at least about four weeks. In some aspects, the dosing interval is at least about a month. In some aspects, the dosing is given intravenously and the dosing interval is at least about three weeks. In some aspects, the dosing is given intravenously and the dosing interval is at least about two weeks. In some aspects, the dosing is given intravenously and the dosing interval is at least about four weeks or about a month. In some aspects, the dosing is given subcutaneously and the dosing interval is at least about three weeks. In some aspects, the dosing is given subcutaneously and the dosing interval is at least about two weeks. In some aspects, the dosing is given subcutaneously and the dosing interval is at least about four weeks or about a month.

In some aspects, the dosing interval (e.g., subcutaneous or intravenous) is about one day, about two days, about three days, about four days, about five days, or about six days. In some aspects, the dosing interval (e.g., subcutaneous or intravenous) is about a week, about two weeks, about three weeks, about four weeks, about a month, about five weeks, about six weeks, about seven weeks, about eighth weeks, about two months, about nine weeks, about 10 weeks, about 11 weeks, about 12 weeks, or about three months. In some aspects, the dosing interval is about three weeks. In some aspects, the dosing interval is about two weeks. In some aspects, the dosing interval is about four weeks. In some aspects, the dosing interval is about a month. In some aspects, the dosing is given intravenously and the dosing interval is about three weeks. In some aspects, the dosing is given intravenously and the dosing interval is about two weeks. In some aspects, the dosing is given intravenously and the dosing interval is about four weeks or a month. In some aspects, the dosing is given subcutaneously and the dosing interval is about three weeks. In some aspects, the dosing is given subcutaneously and the dosing interval is about two weeks. In some aspects, the dosing is given subcutaneously and the dosing interval is about four weeks or about a month.

In some aspects, the present method comprises administering multiple doses of a fusion protein described herein to a subject in need thereof, wherein the multiple doses are administered via two or more different routes, e.g., one dose administered intravenously and another administered subcutaneously. In some aspects, the present method provides (i) intravenously administering a first dose of a fusion protein to a subject in need thereof and (ii) subcutaneously administering a second (or a final) dose of the fusion protein to the subject in need thereof. In some aspects, the present method provides (i) intravenously administering one or more doses of a fusion protein to a subject in need thereof and (ii) subcutaneously administering one or more doses of the fusion protein to the subject. In some aspects, the dosing interval and/or the dosages can be adjusted between the intravenous administration and the subcutaneous administration.

In some aspects, the present method further comprises administering to the subject a corticosteroid. In some aspects, the corticosteroid is selected from the group consisting of: prednisolone, methylprednisolone, prednisone, hydrocortisone, dexamethasone, betamethasone, budesonide, triamcinolone, cortisone, desoxycorticosterone, fludrocortisone, and paramethasone. In some aspects, the corticosteroid is prednisolone, methylprednisolone, or prednisone. In some aspects, the corticosteroid is prednisolone.

In some aspects, the corticosteroid is administered to the subject via a topical, epidermal, mucosal, intranasal, oral, vaginal, rectal, sublingual, topical, intravenous, intraperitoneal, intramuscular, inaarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural, or intrasernal route. In some aspects, the corticosteroid is administered to the subject via a topical, oral, intravenous, or intramuscular route.

In some aspects, the corticosteroid is administered before, concurrently with, or after each dose of the of the fusion protein. In some aspects, two or more doses of the corticosteroid are administered to the subject at a dosing interval between each dose. In some aspects, the dosing interval of the corticosteroid is at least about one day, at least about two days, at least about three days, at least about four days, at least about five days, at least about six days, at least about one week, at least about two weeks, at least about three weeks, at least about four weeks, at least about a month, at least about five weeks, at least about six weeks, at least about seven weeks, at least about eight weeks, at least about two months, at least about nine weeks, at least about ten weeks, at least about eleven weeks, at least about twelve weeks, or at least about three months. In some aspects, the corticosteroid is prednisoloine, wherein the fusion protein is administered to the subject subcutaneously twice a week, and wherein the prednisolone is administered to the subject orally three times a week.

### II.B. Diseases and Disorders

Subjects suitable for the present methods include human patients in whom enhancement of an immune response would be desirable. The methods are particularly suitable for treating human patients having a disease or disorder that can be treated by augmenting an immune response (e.g., a T-cell mediated immune response, e.g., an antigen specific T cell response). In some aspects, administering the amount of the IL2 fusion protein to the subject modifies an immune response in the subject. In some aspects, the immune response is enhanced, stimulated, or up-regulated in the subject.

Given the ability of IL2 fusion proteins described herein to stimulate or co-stimulate T cell responses, e.g., antigen-specific T cell responses, such as by inhibiting negative effects of IL2 or IL2Rα, provided herein are in vitro and in vivo methods of using the IL2 fusion proteins described herein to stimulate, enhance or upregulate antigen-specific T cell responses. In some aspects, CD3 stimulation is also provided (e.g., by coincubation with a cell expressing membrane CD3), which stimulation can be provided at the same time, before, or after stimulation with an IL2 fusion protein described herein.

Any suitable indicator of an antigen-specific T cell response can be used to measure the antigen-specific T cell response. Non-limiting examples of such suitable indicators include increased T cell proliferation in the presence of the antibody and/or increase cytokine production in the presence of the antibody. In some aspects, interleukin-2 and/or interferon-7 production by the antigen-specific T cell is stimulated.

T cells that can be enhanced or co-stimulated with IL2 fusion proteins described herein include CD4⁺ T cells and CD8⁺ T cells. The T cells can be Teff cells, e.g., CD4⁺ Teff cells, CD8⁺ Teff cells, Thelper (Th) cells (e.g., Th1 cells) or T cytotoxic (Tc) cells.

In some aspects, the disease or disorder is an infectious disease or an immune-mediated disease. Treatment of a subject having a disease or disorder with an IL2 fusion protein described herein can result in, e.g., stable disease, partial response, increased overall survival, increased disease free survival, or enhanced progression free survival.

In some aspects, the immune-mediated disease is an inflammatory disease or an autoimmune disease. There is much interest to harness the suppressive power of Tregs to inhibit unwanted immune responses. Data in mouse and man shows that enhancing IL2R signaling with a low dose of IL2 selectively boosts Tregs and enhances immune tolerogenic mechanisms. IL2 fusion proteins provided herein represent a new and improved form of IL2 that more potentially enhances Tregs. Thus, the IL2 fusion proteins can be administered to patients with autoimmune diseases, chronic graft versus host disease, transplant rejection reactions, and other conditions where the goal is to suppress self-reactivity.

In some aspects, the immune-mediated disease is selected from the group consisting of: type 1 diabetes; multiple sclerosis; rheumatoid arthritis; celiac disease; systemic lupus erythematosus; lupus nephritis; cutaneous lupus; juvenile idiopathic arthritis; Crohn's disease; ulcerative colitis; systemic sclerosis; graft versus host disease (GvHD); psoriasis; alopecia areata; HCV-induced vasculitis; Sjogren's syndrome; Pemphigus; Ankylosing Spondylitis; Behcet's Disease; Wegener's Granulomatosis; Takayasu's Disease; Autoimmune Hepatitis; Sclerosing Cholangitis; Gougerot-sjögren; inflammatory bowel disease; Immune Dysregulation, Polyendocrinopathy, Enteropathy, X-linked (IPEX) syndrome; and Macrophage Activation Syndrome. In some aspects, the immune-mediated disease is systemic lupus erythematosus, lupus nephritis, or cutaneous lupus. In some aspects, the immune-mediated disease is systemic lupus erythematosus.

In some aspects, an IL2 fusion protein disclosed herein is administered to patients having an inflammatory disease or an autoimmune disease that exhibited an inadequate response to, or progressed on, a prior treatment. In some aspects, an IL2 fusion protein disclosed herein is administered to patients who have not previously received (i.e., been treated with) treatment for the an inflammatory disease or an autoimmune disease

In some aspects, an IL2 fusion protein disclosed herein is administered with a standard of care treatment for an inflammatory disease or an autoimmune disease. In some aspects, an IL2 fusion protein disclosed herein is administered as a maintenance therapy for an inflammatory disease or an autoimmune disease, e.g., a therapy that is intended to prevent the occurrence or recurrence of inflammation.

In some aspects, an IL2 fusion protein disclosed herein is administered as a monotherapy for treatment of an inflammatory disease or an autoimmune disease, or as the only immuno-stimulating therapy for treatment of an inflammatory disease or an autoimmune disease. In some aspects, an IL2 fusion protein disclosed herein is combined with a vaccination protocol for treatment of an inflammatory disease or an autoimmune disease. In some aspects, an IL2 fusion protein disclosed herein is combined with an antibody used for treatment of an inflammatory disease or an autoimmune disease.

In some aspects, an IL2 fusion protein disclosed herein is combined with a corticosteroid used for treatment of an inflammatory disease or an autoimmune disease.

In some aspects, an IL2 fusion protein disclosed herein is combined with a corticosteroid used for treatment of systemic lupus erythematosus. In some aspects, the corticosteroid used for treatment of systemic lupus erythematosus is prednisolone, methylprednisolone, prednisone, hydrocortisone, dexamethasone, betamethasone, budesonide, triamcinolone, cortisone, desoxycorticosterone, fludrocortisone, or paramethasone. In some aspects, the corticosteroid used for treatment of systemic lupus erythematosus is prednisolone, methylprednisolone, or prednisone. In some aspects, the corticosteroid used for treatment of systemic lupus erythematosus is prednisolone.

In some aspects, an IL2 fusion protein disclosed herein is combined with a corticosteroid used for treatment of lupus nephritis. In some aspects, the corticosteroid used for treatment of systemic lupus nephritis is prednisolone, methylprednisolone, prednisone, hydrocortisone, dexamethasone, betamethasone, budesonide, triamcinolone, cortisone, desoxycorticosterone, fludrocortisone, or paramethasone. In some aspects, the corticosteroid used for treatment of lupus nephritis is prednisolone, methylprednisolone, or prednisone. In some aspects, the corticosteroid used for treatment of lupus nephritis is prednisolone.

In some aspects, an IL2 fusion protein disclosed herein is combined with a corticosteroid used for treatment of cutaneous lupus. In some aspects, the corticosteroid used for treatment of cutaneous lupus is prednisolone, methylprednisolone, prednisone, hydrocortisone, dexamethasone, betamethasone, budesonide, triamcinolone, cortisone, desoxycorticosterone, fludrocortisone, or paramethasone.

In some aspects, an IL2 fusion protein described herein is not significantly toxic. For example, an IL2 fusion protein described herein is not significantly toxic to an organ of a human, e.g., one or more of the liver, kidney, brain, lungs, and heart, as determined, e.g., in clinical trials. In some aspects, an IL2 fusion protein described herein does not significantly trigger an undesirable immune response, e.g., autoimmunity or inflammation.

In some aspects, treatment of a subject with an IL2 fusion protein described herein does not result in overstimulation of the immune system to the extent that the subject's immune system then attacks the subject itself (e.g., autoimmune response) or results in, e.g., anaphylaxis. Thus, in some aspects, the IL2 fusion proteins described herein do not cause anaphylaxis.

In some aspects, treatment of a subject with an IL2 fusion protein described herein does not cause significant inflammatory reactions, e.g., immune-mediated pneumonitis, immune-mediated colitis, immune mediated hepatitis, immune-mediated nephritis or renal dysfunction, immune-mediated hypophysitis, immune-mediated hypothyroidism and hyperthyroidism, or other immune-mediated adverse reactions. In some aspects, treatment of a subject with the IL2 fusion proteins described herein does not cause significant cardiac disorders, e.g., ventricular arrhythmia; eye disorders, e.g., iridocyclitis; infusion-related reactions; increased amylase, increased lipase; nervous system disorders, e.g., dizziness, peripheral and sensory neuropathy; skin and subcutaneous tissue disorders, e.g., rash, pruritus, exfoliative dermatitis, erythema multiforme, vitiligo or psoriasis; respiratory, thoracic and mediastinal disorders, e.g., cough; fatigue; nausea; decreased appetite; constipation; arthralgia; or diarrhea.

The disclosure further provides compositions for use in accordance with any method disclosed herein.

### II. C. IL2 Fusion Proteins

The IL2 fusion proteins administered in the present methods comprise at least two components: (a) a first polypeptide comprising an Interleukin-2 (IL2) polypeptide; and (b) a second polypeptide comprising an extracellular domain of an Interleukin-2 Receptor alpha (IL2Rα) polypeptide; wherein the extracellular domain of the IL2Rα polypeptide has at least one fewer glycosylation compared to the extracellular domain of native IL2Rα (SEQ ID NO: 1); and/or (ii) the IL2 polypeptide has at least one fewer glycosylation compared to native IL2 (SEQ ID NO: 2). In some aspects, the fusion protein has IL2 activity.

Fusion proteins described herein specifically bind human IL2R, and more specifically, a particular domain (e.g., a functional domain) within the extracellular domain of human IL2Rα. In some aspects, the fusion protein comprising IL2 is an antagonist. In some aspects, the fusion protein comprising IL2 binds to human IL2Rα with high affinity.

Multiple receptor subunits contribute to effective IL-2 receptor signaling. IL-2Rβ and common gamma chain receptors (IL-2Rγ) make up the signaling components of the receptor and are both necessary and sufficient for IL-2 signaling. Activation of the IL-2Rβγ heterodimeric receptor leads to recruitment of JAK1 and JAK3, activation of PI3K and ultimately phosphorylation of STAT5. See, for example, Malek, T. R., Annu Rev Immunol. 26:453-79 (2008). IL-2Rβ and IL-2Rγ are expressed on all IL-2 sensitive immune cells: Tregs, Tconv, CD8 T cells, NK cells and innate lymphoid cells type 2 (ILC2), whereas the alpha subunit, IL-2Rα or CD25, has a more restricted expression. CD25 is constitutively expressed on Tregs, has been reported on ILC2s, and is only transiently expressed on activated T cells, B cells, and NK cells. See, for example, Simoni, Y., et al., Immunity 46(1):148-61 (2017). CD25 has a moderate (˜25 nM) affinity for IL-2 and does not directly participate in signaling. See, for example, Rickert, M., et al., Science 308:1477-80 (2005).

Based on the crystal structure of quaternary complex of IL-2 and the extracellular domains of all 3 receptor components, CD25 does not appear to make direct contact with IL-2Rβ or IL-2Rγ. See, for example, Nelson, B. H., et al., Nature 369:333-6 (1994); and Stauber, D. J., et al., Proc. Natl. Acad. Sci. U.S.A. 103:2788-93 (2006). Instead, CD25 appears to serve as a cell surface sink for IL-2 that increases the apparent potency of IL-2 for cells on which CD25 is expressed at high levels on the same cell as the IL-2Rβ and IL-2Rγ subunits. See, for example, Pillet, A. H., et al., J Mol Biol. 403:671-92 (2010). Constitutively high CD25 expression on Tregs confers very high sensitivity to IL-2, with significantly greater potency of IL-2 on Treg relative to other non-regulatory T cells. See, for example, Dupont, G., et al., Cytokine 69:146-9 (2014). IL-2 treatment of Tregs leads to robust proliferation and activation including up-regulation of CD25 and FoxP3, as well as other genes associated with Treg suppressive activity. See, for example, Sakaguchi, S., et al., J Immunol. 155(3):1151-64 (1995). In contrast to Treg, effector T cells and most NK cells require higher IL-2 levels for activation as they lack high constitutive CD25 expression and only transiently upregulate CD25 upon activation. See, for example, Letourneau, S., et al., J Allergy Clin Immunol. 123(4):758-62 (2009).

### II. C.1 IL2 Polypeptides of the IL2 Fusion Proteins

The IL2 fusion comprises a first polypeptide comprising an Interleukin-2 (IL2) polypeptide.

In some aspects, the IL2 polypeptide of the IL2 fusion protein is a native or recombinant IL2 from any vertebrate source, including mammals such as primates (e.g., humans) and rodents (e.g., mice and rats), and domesticated or agricultural mammals unless otherwise indicated.

The term IL2 encompasses unprocessed IL2, as well as, any form of IL2 that results from processing in the cell (i.e., the mature form of IL2). The term also encompasses naturally occurring variants and fragments of IL2, e.g., splice variants or allelic variants, and non-naturally occurring variants. The amino acid sequence of an exemplary mature form of human IL2 (having the 20 amino acid signal sequence) is shown in SEQ ID NO: 2. Unprocessed human IL2 additionally comprises an N-terminal 20 amino acid signal peptide (SEQ ID NO: 7), which is absent in the mature IL2 molecule. The amino acid sequence of an exemplary mature form of mouse IL2 (having the 20 amino acid signal sequence) is shown in SEQ ID NO: 8. Unprocessed mouse IL2 additionally comprises an N-terminal 20 amino acid signal peptide (SEQ ID NO: 9), which is absent in the mature IL2 molecule. By a “native IL2,” also termed “wild-type IL2,” is meant a naturally occurring or recombinant IL2.

Additional nucleic acid and amino acid sequences for IL2 are known. See, for example, GenBank Accession Nos: Q7JFM2 (Aotus lemurinus (Gray-bellied night monkey)); Q7JFM5 (Aotus nancymaae (Ma's night monkey)); P05016 (Bas taurus (Bovine)); Q29416 (Canis familiaris (Dog) (Canis lupus familiaris)); P36835 (Capra hircus (Goat)); and, P37997 (Equus caballus (Horse).

In some aspects, the first polypeptide of the fusion protein comprises an amino acid sequence at least about 60%, at least about 70%, at least about 80%, at least about 85%, at least about 90%, at least about 95%, at least about 96%, at least about 97%, at least about 98%, at least about 99% or about 100% identical to SEQ ID NO: 2.

Biologically active fragments and variants of IL2 are also provided. Such IL2 active variants or fragments will retain IL2 activity. Biological activity of IL2 can refer to the ability to stimulate IL2 receptor bearing lymphocytes. Such activity can be measured both in vitro and in vivo. IL2 is a global regulator of immune activity and the effects seen here are the sum of such activities. For example, it is regulates survival activity (Bcl-2), induces T effector activity (IFN-gamma, Granzyme B, and Perforin), and promotes T regulatory activity (FoxP3). See, for example, Malek et al., Immunity 33(2):153-65 (2010).

Biologically active variants of IL2 are known. See, for example, U.S. Publication Nos. 2006/0269515 and 2006/0160187 and WO/1999/060128.

Biologically active fragments and variants of IL2 can be employed in the fusion proteins disclosed herein. Such a functional fragment can comprise at least 10, 11, 12, 13, 14, 15, 16, 17, 18, 19, 20, 30, 40, 50, 75, 100, 125, 150 or more continuous amino acids of SEQ ID NO: 2. Alternatively, a functional variant can comprise at least 75%, 80%, 85%, 90%, 91%, 92%, 93%, 94%, 95%, 96%, 97%, 98%, or 99% sequence identity to the sequence set forth in SEQ ID NO: 2.

Active variants and fragments of polynucleotides encoding the IL2 proteins are further provided. Such polynucleotide can comprise at least 100, 200, 300, 400, 500, 600, 700 continuous nucleotides of polypeptide encoding SEQ ID NO: 2, and continue to encode a protein having IL2 activity. Alternatively, a functional polynucleotide can comprise at least 75%, 80%, 85%, 90%, 91%, 92%, 93%, 94%, 95%, 96%, 97%, 98%, or 99% sequence identity to the polypeptide encoding the amino sequence set forth in SEQ ID NO: 2 and continue to encode a functional IL2 polypeptide.

Exemplary polypeptide sequences of IL2 are recited in Table 1, below.

TABLE 1

| SEQ | | |
|---|---|---|
| ID | | |
| NO | Description | Sequence |
| 7 | IL2 (human, | MYRMQLLSCIALSLALVTNSAPTSSSTKKT |
| | unprocessed) | QLQLEHLLLDLQMILNGINNYKNPKLTRML |
| | | TFKFYMPKKATELKHLQCLEEELKPLEEVL |
| | | NLAQSKNFHLRPRDLISNINVIVLELKGSE |
| | | TTFMCEYADETATIVEFLNRWITFCQSIIS |
| | | TLT |
| 2 | IL2 (human, | APTSSSTKKTQLQLEHLLLDLQMILNGINN |
| | mature form) | YKNPKLTRMLTFKFYMPKKATELKHLQCLE |
| | | EELKPLEEVLNLAQSKNFHLRPRDLISNIN |
| | | VIVLELKGSETTFMCEYADETATIVEFLNR |
| | | WITFCQSIISTLT |
| 9 | IL2 (mouse, | MYSMQLASCVTLTLVLLVNSAPTSSSTSSS |
| | unprocessed) | TAEAQQQQQQQQQHLEQLLMDLQELLSRME |
| | | NYRNLKLPRMLTFKFYLPKQATELKDLQCL |
| | | EDELGPLRHVLDLTQSKSFQLEDAENFISN |
| | | IRVTVVKLKGSDNTFECQFDDESATVVDFL |
| | | RRWIAFCQSIISTSPQ |
| 8 | IL2 (mouse, | APTSSSTSSSTAEAQQQQQHLEQLLMDLQE |
| | mature form) | LLSRMENYRNLKLPRMLTFKFYLPKQATEL |
| | | KDLQCLEDELGPLRHVLDLTQSKSFQLEDA |
| | | ENFISNIRVTVVKLKGSDNTFECQFDDESA |
| | | TVVDFLRRWIAFCQSIISTSPQ |

In some aspects, the IL2 polypeptide has at least one fewer glycosylation site compared to native IL2 (SEQ ID NO: 2). In some aspects, the at least one fewer glycosylation sites is due to one or more mutations that removes a glycosylation.

In some aspects, the fusion protein comprises a mutation that is a substitution of an amino acid having a glycosylation site with an amino acid not having a glycosylation site. In some aspects, the mutation removes an O-glycosylation and/or an N-glycosylation. In one aspect, the mutation removes an O-glycosylation, e.g., threonine at amino acid 3 of SEQ ID NO: 2. In another aspect, the mutation removes an N-glycosylation.

In some aspects, the mutation is one or more substitutions of an amino acid of IL2 that is glycosylated with an amino acid that is not glycosylated. In some aspects, the mutation is one or more substitutions of an amino acid of IL2 that allows glycosylation at a nearby amino acid with an amino acid that does not allow glycosylation at the nearby amino acid.

In some aspects, the one or more substitutions of an amino acid of IL2 are from an alanine to an amino acid selected from the group consisting of arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine.

In some aspects, the one or more substitutions of an amino acid of IL2 are from a threonine to an amino acid selected from the group consisting of alanine, arginine, asparagine, aspartic acid, cysteine, glutamine, glutamic acid, glycine, histidine, isoleucine, leucine, lysine, methionine, phenylalanine, proline, tryptophan, tyrosine, and valine.

In some aspects, the one or more substitutions of an amino acid of IL2 are from a reactive amino acid, e.g., a cysteine, to an amino acid selected from the group consisting of alanine, arginine, asparagine, aspartic acid, glutamine, glutamic acid, glycine, histidine, isoleucine, leucine, lysine, methionine, phenylalanine, proline, serine, threonine, tryptophan, tyrosine, and valine. In some aspects, the one or more substitutions are from a cysteine to a serine. In some aspects, the one or more substitutions are from a cysteine to an alanine. In some aspects, the one or more substitutions are from a cysteine to a valine.

In some aspects, the one or more substitutions are at amino acid T3 of IL2 compared to corresponding to SEQ ID NO: 2.

In some aspects, the one of the substitutions is at amino acid C125 of SEQ ID NO: 2. In one aspect, the substitution at amino acid C125 is selected from the group consisting of C125S, C125A, and C125V.

In some aspects, the mutation is a deletion. In some aspects, the deletion is at amino acid A1 of SEQ ID NO: 2.

The present disclosure also includes any other mutations to the IL2 polypeptide. In other aspects, the mutations also include one or more substitutions that improve the properties of IL2, e.g., improve IL2 activity, improve a half-life of IL2, improve stability, etc.

As disclosed below in this section, the mutations recited herein are mutations relative to amino acid positions of SEQ ID NO: 2. According to the present invention, any of the mutations below alone or in combination with the other disclosed mutations or any known in the art could be used in one or more of the IL2 fusion proteins as described herein.

In some aspects, IL2 comprises one or more mutations disclosed in Carmenate et al., J Immunol, 200(10):3475-84 (2018) and/or in U.S. Pat. No. 8,759,486: for example, at amino acid residue Q22, Q126, 1129, S130, or any combination thereof, e.g., Q22V, Q126A, I129D, S130G, or any combination thereof. In some aspects, IL2 comprises one or more mutations of L18N, Q126Y, and S130R as disclosed in U.S. Pat. No. 8,759,486 B2. In some aspects, IL2 comprises one or more mutations of Q13Y, Q126Y, 1129D, and S130R as disclosed in U.S. Pat. No. 8,759,486 B2. In some aspects, IL2 comprises one or more mutations of K35E, K35D, and K35Q as disclosed in WO 2018/091003 A1.

In some aspects, IL2 comprises one or more mutations disclosed in Epstein et al. Blood, 101(12):4853-61 (2003) and/or in U.S. Pat. No. 7,371,371: for example, at amino acid residue R38, e.g., R38W. In some aspects, IL2 comprises the mutation of R38W and one or more mutations outside of amino acid positions 22 to 58 of IL2 as disclosed in U.S. Pat. No. 7,371,371 B2.

In some aspects, IL2 comprises one or more mutations disclosed in Wittrup et al. J Immunother. 32(9):887-94 (2009) and/or in U.S. Pat. No. 8,906,356: for example, amino acid residue 91, 126, or both, e.g., V91R, Q126T, or both. In some aspects, IL2 comprises the mutation of E15W as disclosed in Wittrup et al. J Immunother. 32(9):887-94 (2009) and also in U.S. Pat. No. 8,906,356. In some aspects, IL2 comprises one or both mutations of N88R and V91R as disclosed in Wittrup et al. J Immunother. 32(9):887-94 (2009) and also in U.S. Pat. No. 8,906,356. In some aspects, IL2 comprises the mutation of Q126T or Q126I as disclosed in Wittrup et al. J Immunother. 32(9):887-94 (2009) and/or in U.S. Pat. No. 8,906,356.

In some aspects, IL2 comprises one or more mutations disclosed in U.S. Pat. No. 8,906,356 B2: for example at amino acid 69, 74, 91, 126, or any combination thereof. In some aspects, the mutation is V91R, Q126T, Q126L, Q127T, or any combination thereof as disclosed in U.S. Pat. No. 8,906,356 B2.

In some aspects, IL2 comprises one or more mutations disclosed in Wittrup et al., J Immunother 32(9):887-94 (2009) and/or in U.S. Pat. No. 7,569,215 B2: for example, at amino acid residue E15, N30, E68, V69, N71, S75, N90, or any combination thereof, e.g., N30S, E68D, V69A, N71A, S75P, N90H, or any combination thereof. In some aspects, IL2 comprises the mutation of E15W as disclosed in Wittrup et al., Biochemistry 44(31) (2005). In some aspects, the mutation is V69A as disclosed in U.S. Pat. No. 7,569,215 B2.

In some aspects, IL2 comprises one or more mutations disclosed in Wittrup et al., J Immunother. 32(9):887-94 (2009) and/or in U.S. Pat. No. 7,951,360 B2: for example, at amino acid residue N29, Y31, K35, T37, K48, V69, N71, N88, or any combination thereof, e.g., N29S, Y31H, K35R, T37A, K48E, V69A, N71R, N88D, or any combination thereof. In some aspects, IL2 comprises the mutation of E15W as disclosed in Wittrup et al., Biochemistry 44(31) (2005).

In some aspects, IL2 comprises one or more mutations disclosed in Wittrup et al., J Immunother. 32(9):887-94 (2009) and/or in U.S. Pat. No. 8,349,311 B2: for example, at amino acid 69, 74, 128, or any combination thereof, e.g., V69A, I128P, or any combination thereof.

In some aspects, IL2 comprises one or more mutations disclosed in Wittrup et al., J Immunother. 32(9):887-94 (2009): for example, at amino acid residue S4, T10, Q11, V69, N88, T133, or any combination thereof, e.g., S4P, T10A, Q11R, V69A, N88D, T133A, or any combination thereof.

In some aspects, IL2 comprises one or more mutations disclosed in Wittrup et al., J Immunother. 32(9):887-94 (2009): for example, at amino acid residue N30, V69, 1128, or any combination thereof, e.g., N30S, V69A, I128T, or any combination thereof.

In some aspects, IL2 comprises one or more mutations disclosed in Wittrup et al., J Immunother. 32(9):887-94 (2009): for example, at amino acid residue K8, Q13, N26, N30, K35, T37, V69, or any combination thereof, e.g., K8R, Q13R, N26D, N30T, K35R, T37R, V69A, or any combination thereof.

In some aspects, IL2 comprises one or more mutations disclosed in Shanafelt et al., Nat Biotechnol. 18(11):1197-202 (2000) for example, at amino acid residue N88, e.g., N88R.

In some aspects, IL2 comprises one or more mutations disclosed in U.S. Pat. No. 9,616,105 B2: for example, amino acids 20, 88, 126, or any combination thereof, e.g., N88R, N88G, or N88I. In some aspects, IL2 comprises a mutation of N88R, N88G, or N88I as disclosed in U.S. Pat. No. 9,616,105 B2. In some aspects, IL2 comprises a mutation of D20H, D20I, or D20Y as disclosed in U.S. Pat. No. 9,616,105 B2. In some aspects, IL2 comprises the mutation of Q126L as disclosed in U.S. Pat. No. 9,616,105 B2.

In some aspects, IL2 comprises one or more mutations disclosed in U.S. Publ. No. 2018/0125941 A1: for example, D20H, N88I, N88G, N88R, Q126L, Q126F, or any combination thereof. In some aspects, IL2 comprises one or more mutations of T3A, N88G, N88R, D20H, C125S, Q126L, and Q126F as disclosed in U.S. Publ. No. 2018/0037624 A1.

In some aspects, IL2 comprises one or more mutations disclosed in U.S. Publ. No. 2017/0327555 A1: for example, at amino acid residue N88, D20, C125, Q126, or any combination thereof, e.g., N88G, N88R, D20H, C125S, Q126L, Q126F, or any combination thereof.

In some aspects, IL2 comprises one or more mutations disclosed in WO 2016/025385 A1: for example, at amino acid residue D109, C125, or both, e.g., D109C, C125S, or both. In some aspects, IL2 comprises one or more mutations disclosed in WO 2016/025385 A1: for example; at amino acid residue D20, N88, Q126, C125, Q126, or any combination thereof, e.g., D20H, N88I, N88G, N88R, Q126L, C125S, Q126F, or any combination thereof.

In some aspects, IL2 comprises one or more mutations disclosed in WO 2016/164937 A1: for example, at amino acid residue L12, Q13, E15, H16, L19, D20, M23, D84, S87, N88, V91, E95, or any combination thereof, e.g., L12G, L12K, L12Q, L12S, Q13G, E15A, E15G, E15S, H16A, H16D, H16G, H16K, H16M, H16N, H16R, H16S, H16T, H16V, H16Y, L19A, L19D, L19E, L19G, L19N, L19S, L19T, L19V, D20A, D20E, D20F, D20G, D20T, D20W, M23R, D84A, D84E, D84G, D84I, D84M, D84Q, D84R, D84S, D84T, S87E, N88A, N88D, N88E, N88F, N88G, N88M, N88R, N88S, N88V, N88W, V91D, V91E, V91G, V91S, E95G, or any combination thereof.

In some aspects, IL2 comprises one or more mutations disclosed in U.S. Pat. Nos. 9,932,380 B2 or 9,580,486: for example, at amino acid residue V91, e.g., V91K. In some aspects, IL2 further comprises a mutation of C125A or C125S. In some aspects, IL2 further comprises a mutation at T3. In some aspects, the mutation at T3 is one of T3A or T3N. In some aspects, IL2 comprises a mutation at S5. In some aspects, the mutation is S5T.

In some aspects, IL2 comprises one or more mutations disclosed in U.S. Pat. No. 9,732,134 B2: for example, E15, H16, Q22, D84, N88, E95, or any combination thereof.

In some aspects, IL2 comprises one or more mutations disclosed in U.S. Publ. No. 2015/0218260 A1: for example, N88D. In some aspects, IL2 comprises a mutation disclosed in U.S. Pat. No. 9,266,938 B2: for example, at amino acid 42, 45, 72, or any combination thereof, e.g., L72G, L72A, L72S, L72T, L72Q, L72E, L72N, L72D, L72R, or L72K. In some aspects, IL2 comprises a mutation of F42A, F42G, F42S, F42T, F42Q, F42E, F42N, F42D, F42R, and F42K. In some aspects, IL2 comprises a mutation of Y45A, Y45G, Y45S, Y45T, Y45Q, Y45E, Y45N, Y45D, Y45R, and Y45K.

In some aspects, IL2 comprises one to four mutations: the first mutation of L72G, L72A, L72S, L72T, L72Q, L72E, L72N, L72D, L72R, or L72K, the second mutation of F42A, F42G, F42S, F42T, F42Q, F42E, F42N, F42D, F42R, or F42K, Y45A, Y45G, Y45S, Y45T, Y45Q, Y45E, Y45N, Y45D, Y45R, or Y45K, the third mutation of T3A, T3G, T3Q, T3E, T3N, T3D, T3R, T3K, or T3P, and/or the fourth mutation of C125A, C125S, C125T or C125V. The mutations listed herein or disclosed in the patents, patent publications or any other references cited herein are incorporated herein by reference in their entireties.

### II. C.2. ILR2α Polypeptides of the IL2 Fusion Proteins

The fusion protein comprises a second polypeptide comprising the extracellular domain of the Interleukin-2 Receptor Alpha (IL2Rα).

In some aspects, the extracellular domain of IL2Rα comprises the amino acid sequence set forth as SEQ ID NO: 1. In some aspects, the second polypeptide comprises an amino acid sequence at least about 60%, at least about 70%, at least about 80%, at least about 85%, at least about 90%, at least about 95%, at least about 96%, at least about 97%, at least about 98%, at least about 99%, or about 100% identical to SEQ ID NO: 1.

In some aspects, the second polypeptide comprises an amino acid sequence at least about 60%, at least about 70%, at least about 80%, at least about 85%, at least about 90%, at least about 95%, at least about 96%, at least about 97%, at least about 98%, at least about 99%, or about 100% identical to SEQ ID NO: 3.

The term “CD25” or “IL2 receptor a,” “IL2Rα,” “IL2Rα,” “IL2-Rα,” and “IL2-Ra” as used herein, refers to any native or recombinant IL2Rα from any vertebrate source, including mammals such as primates (e.g., humans) and rodents (e.g., mice and rats) and domesticated or agricultural mammals unless otherwise indicated. The term also encompasses naturally occurring variants of IL2Rα, e.g., splice variants or allelic variants, or non-naturally occurring variants. Human IL2 exerts its biological effects via signaling through its receptor system, IL2R. IL2 and its receptor (IL2R) are required for T-cell proliferation and other fundamental functions which are crucial of the immune response. IL2R consists of 3 non-covalently linked type I transmembrane proteins which are the alpha (p55), beta (p75), and gamma (p65) chains. The human IL2R alpha chain contains an extracellular domain of 219 amino acids, a transmembrane domain of 19 amino acids, and an intracellular domain of 13 amino acids. The secreted extracellular domain of IL2R alpha (IL2Rα) can be employed in the fusion proteins describe herein.

The amino acid sequence of an exemplary mature form of human IL2Rα is shown in SEQ ID NO: 10. Unprocessed human IL2Rα is shown in SEQ ID NO: 11. The extracellular domain of SEQ ID NO: 11 and/or SEQ ID NO: 10 is set forth in SEQ ID NO: 1. The amino acid sequence of an exemplary mature form of mouse IL2Rα is shown in SEQ ID NO: 12. Unprocessed mouse IL2Rα is shown in SEQ ID NO: 13. The extracellular domain of SEQ ID NO: 13 and/or SEQ ID NO: 12 is set forth in SEQ ID NO: 14. By a “native IL2Rα”, also termed “wild-type IL2Rα”, is meant a naturally occurring or recombinant IL2Rα.

Nucleic acid and amino acid sequences for IL2Rα are known. See, for example, GenBank Accession Nos: NP_001030597.1 (P. troglodytes); NP_001028089.1 (M. mulatta); NM_001003211.1 (C. lupus); NP_776783.1 (B. taurus); NP_032393.3 (M. musculus); and, NP_037295.1 (R. norvegicus).

The extracellular domain of IL2Rα as used herein means a functional IL2Rα extracellular (EC) domain in its normal role in binding to IL2, unless otherwise specified. The term “IL2Rα EC domain” includes a functional fragment, variant, analog, or derivative thereof that retains the function of full-length wild-type IL2Rα EC in IL2 binding. The IL2Rα EC domain can be the human, porcine, canine, rat, or murine IL2Rα EC domain. The phrase “biological activity of the IL2Rα EC domain” refers to one or more of the biological activities of EC domain of IL2Rα, including but not limited to, the ability to enhance intracellular signaling in IL2 receptor responsive cells. Non-limiting examples of biologically active fragments and variants of the IL2Rα EC domain are disclosed, for example, in Robb et al., Proc. Natl. Acad. Sci. USA, 85:5654-8 (1988). In some aspects, the biologically active fragments and variants of the IL2Rα EC domain disclosed herein comprise at least one fewer glycosylation compared to the extracellular domain of native IL2Rα.

Biologically active fragments and variants of the extracellular domain of IL2Rα can be employed in the fusion proteins disclosed herein. Such a functional fragment can comprise at least 10, 11, 12, 13, 14, 15, 16, 17, 18, 19, 20, 30, 40, 50, 75, 100, 125, 150, 175, 200, 215 or greater continuous amino acids of the extracellular domain of any one of SEQ ID NO: 1. Alternatively, a functional variant can comprise at least 75%, 80%, 85%, 90%, 91%, 92%, 93%, 94%, 95%, 96%, 97%, 98%, or 99% sequence identity to the sequence set forth in SEQ ID NO: 1.

Active variants and fragments of polynucleotides encoding the extracellular domain of IL2Rα are further provided. Such polynucleotide can comprise at least 100, 200, 300, 400, 500, 600 or greater continuous nucleotides of polypeptide encoding SEQ ID NO: 1 and continue to encode a protein having the extracellular domain activity of IL2Rα. Alternatively, a functional polynucleotide can comprise at least 80%, 85%, 90%, 91%, 92%, 93%, 94%, 95%, 96%, 97%, 98%, or 99% sequence identity to the polypeptide encoding the amino sequence set forth in SEQ ID NO: 1 and continue to encode a protein having the extracellular domain activity of IL2Rα.

Exemplary polypeptide sequences of IL2Rα are recited in Table 2.

TABLE 2

| SEQ | | |
|---|---|---|
| ID | | |
| NO | Description | Sequence |
| 11 | IL2Rα (human, | MDSYLLMWGLLTFIMVPGCQAELCD |
| | unprocessed form) | DDPPEIPHATFKAMAYKEGTMLNCE |
| | | CKRGFRRIKSGSLYMLCTGNSSHSS |
| | | WDNQCQCTSSATRNTTKQVTPQPEE |
| | | QKERKTTEMQSPMQPVDQASLPGHC |
| | | REPPPWENEATERIYHFVVGQMVYY |
| | | QCVQGYRALHRGPAESVCKMTHGKT |
| | | RWTQPQLICTGEMETSQFPGEEKPQ |
| | | ASPEGRPESETSCLVTTTDFQIQTE |
| | | MAATMETSIFTTEYQVAVAGCVFLL |
| | | ISVLLLSGLTWQRRQRKSRRTI |
| 10 | IL2Rα (human, | ELCDDDPPEIPHATFKAMAYKEGTM |
| | mature form) | LNCECKRGFRRIKSGSLYMLCTGNS |
| | | SHSSWDNQCQCTSSATRNTTKQVTP |
| | | QPEEQKERKTTEMQSPMQPVDQASL |
| | | PGHCREPPPWENEATERIYHFVVGQ |
| | | MVYYQCVQGYRALHRGPAESVCKMT |
| | | HGKTRWTQPQLICTGEMETSQFPGE |
| | | EKPQASPEGRPESETSCLVTTTDFQ |
| | | IQTEMAATMETSIFTTEYQVAVAGC |
| | | VFLLISVLLLSGLTWQRRQRKSRRT |
| | | I |
| 1 | IL2Rα (human, | ELCDDDPPEIPHATFKAMAYKEGTM |
| | mature form of | LNCECKRGFRRIKSGSLYMLCTGNS |
| | IL2Rα | SHSSWDNQCQCTSSATRNTTKQVTP |
| | extracellular | QPEEQKERKTTEMQSPMQPVDQASL |
| | domain) | PGHCREPPPWENEATERIYHFVVGQ |
| | | MVYYQCVQGYRALHRGPAESVCKMT |
| | | HGKTRWTQPQLICTGEMETSQFPGE |
| | | EKPQASPEGRPESETSCLVTTTDFQ |
| | | IQTEMAATMETSIFTTEYQ |
| 13 | IL2Rα (mouse, | MEPRLLMLGFLSLTIVPSCRAELCL |
| | unprocessed | YDPPEVPNATFKALSYKNGTILNCE |
| | form) | CKRGFRRLKELVYMRCLGNSWSSNC |
| | | QCTSNSHDKSRKQVTAQLEHQKEQQ |
| | | TTTDMQKPTQSMHQENLTGHCREPP |
| | | PWKHEDSKRIYHFVEGQSVHYECIP |
| | | GYKALQRGPAISICKMKCGKTGWTQ |
| | | PQLTCVDEREHHRFLASEESQGSRN |
| | | SSPESETSCPITTTDFPQPTETTAM |
| | | TETFVLTMEYKVAVASCLFLLISIL |
| | | LLSGLTWQHRWRKSRRTI |
| 12 | IL2Rα (mouse, | ELCLYDPPEVPNATFKALSYKNGTI |
| | mature form) | LNCECKRGFRRLKELVYMRCLGNSW |
| | | SSNCQCTSNSHDKSRKQVTAQLEHQ |
| | | KEQQTTTDMQKPTQSMHQENLTGHC |
| | | REPPPWKHEDSKRIYHFVEGQSVHY |
| | | ECIPGYKALQRGPAISICKMKCGKT |
| | | GWTQPQLTCVDEREHHRFLASEESQ |
| | | GSRNSSPESETSCPITTTDFPQPTE |
| | | TTAMTETFVLTMEYKVAVASCLFLL |
| | | ISILLLSGLTWQHRWRKSRRTI |
| 14 | IL2Rα (mouse, | ELCLYDPPEVPNATFKALSYKNGTI |
| | mature form of | LNCECKRGFRRLKELVYMRCLGNSW |
| | IL2Rα | SSNCQCTSNILRASHDKSRKQVTAQ |
| | extracellular | LEHQKEQQTTTDMQKPTQSMHQENL |
| | domain) | TGHCREPPPWKHEDSKRIYHFVEGQ |
| | | SVHYECIPGYKALQRGPAISICKMK |
| | | CGKTGWTQPQLTCVDEREHHRFLAS |
| | | EESQGSRNSSPESETSCPITTTDFP |
| | | QPTETTAMTETFVLTMEYK |
| 4 | IL2Rα (human, | ELCDDDPPEIPHATFKAMAYKEGTM |
| | mature form of | LNCECKRGFRRIKSGSLYMLCTGNS |
| | IL2Rα | SHSSWDNQCQCTSSATRNTTKQVTP |
| | extracellular | QPEEQKERKTTEMQSPMQPVDQASL |
| | domain)-full- | PGHCREPPPWENEATERIYHFVVGQ |
| | truncated | MVYYQCVQGYRALHRGPAESVCKMT |
| | | HGKTRWTQPQLICTGE |
| 3 | IL2Rα (human, | ELCDDDPPEIPHATFKAMAYKEGTM |
| | mature form of | LNCECKRGFRRIKSGSLYMLCTGNS |
| | IL2Rα | SHSSWDNQCQCTSSATRNTTKQVTP |
| | extracellular | QPEEQKERKTTEMQSPMQPVDQASL |
| | domain)-half- | PGHCREPPPWENEATERIYHFVVGQ |
| | truncated | MVYYQCVQGYRALHRGPAESVCKMT |
| | | HGKTRWTQPQLICTGEMETSQFPGE |
| | | EKPQASPEGRPESETS |

In some aspects, the fusion proteins provided herein can comprise at least one mutation within the EC domain of IL2Rα.

In some aspects, the EC domain of the IL2Rα polypeptide has at least one fewer glycosylation, at least two fewer glycosylations, at least three fewer glycosylations, at least four fewer glycosylations, at least five fewer glycosylations, at least six fewer glycosylations, at least seven fewer glycosylations, at least eight fewer glycosylations, or at least nine fewer glycosylations compared to the extracellular domain of native IL2Rα (SEQ ID NO: 1).

In some aspects, the EC domain of the IL2Rα polypeptide having at least one fewer glycosylation comprises a mutation that removes a glycosylation. In other aspects, the fusion protein comprises a mutation that is a substitution of an amino acid having a glycosylation site with an amino acid not having a glycosylation site. In some aspects, the mutation removes an O-glycosylation and/or an N-glycosylation. In one aspect, the mutation removes an O-glycosylation. In another aspect, the mutation removes an N-glycosylation.

In some aspects, the mutation in the fusion protein comprises a deletion of the C-terminal end of IL2Rα. In some aspects, the mutation is a deletion of amino acids 167 to 219 of SEQ ID NO: 1. In some aspects, the mutation is a deletion of amino acids 168 to 219 of SEQ ID NO: 1. In some aspects, the mutation is a deletion of amino acids 169 to 219 of SEQ ID NO: 1. In some aspects, the mutation is a deletion of amino acids 170 to 219 of SEQ ID NO: 1. In some aspects, the mutation is a deletion of amino acids 171 to 219 of SEQ ID NO: 1. In some aspects, the mutation is a deletion of amino acids 172 to 219 of SEQ ID NO: 1. In some aspects, the mutation is a deletion of amino acids 173 to 219 of SEQ ID NO: 1. In some aspects, the mutation is a deletion of amino acids 174 to 219 of SEQ ID NO: 1. In some aspects, the mutation is a deletion of amino acids 175 to 219 of SEQ ID NO: 1. In some aspects, the mutation is a deletion of amino acids 176 to 219 of SEQ ID NO: 1. In some aspects, the mutation is a deletion of amino acids 177 to 219 of SEQ ID NO: 1. In some aspects, the mutation is a deletion of amino acids 178 to 219 of SEQ ID NO: 1. In some aspects, the mutation is a deletion of amino acids 179 to 219 of SEQ ID NO: 1. In some aspects, the mutation is a deletion of amino acids 180 to 219 of SEQ ID NO: 1. In some aspects, the mutation is a deletion of amino acids 181 to 219 of SEQ ID NO: 1. In some aspects, the mutation is a deletion of amino acids 182 to 219 of SEQ ID NO: 1. In some aspects, the mutation is a deletion of amino acids 183 to 219 of SEQ ID NO: 1. In some aspects, the mutation is a deletion of amino acids 184 to 219 of SEQ ID NO: 1. In some aspects, the mutation is a deletion of amino acids 185 to 219 of SEQ ID NO: 1. In some aspects, the mutation is a deletion of amino acids 186 to 219 of SEQ ID NO: 1. In some aspects, the mutation is a deletion of amino acids 187 to 219 of SEQ ID NO: 1. In some aspects, the mutation is a deletion of amino acids 188 to 219 of SEQ ID NO: 1. In some aspects, the mutation is a deletion of amino acids 189 to 219 of SEQ ID NO: 1. In some aspects, the mutation is a deletion of amino acids 190 to 219 of SEQ ID NO: 1. In some aspects, the mutation is a deletion of amino acids 191 to 219 of SEQ ID NO: 1. In some aspects, the mutation is a deletion of amino acids 192 to 219 of SEQ ID NO: 1.

In some aspects, the mutation is a deletion of amino acids from 167, 168, 169 or 171 through 192 to 219, corresponding to SEQ ID NO: 1. In some aspects, the mutation does not include a deletion of 170 to 219, corresponding to SEQ ID NO: 1.

In some aspects, the second polypeptide comprises, consists essentially of, or consists of an amino acid sequence at least about 80%, at least about 85%, at least about 90%, at least about 95%, at least about 96%, at least about 97%, at least about 98%, at least about 99%, or about 100% identical to the amino acid sequence set forth in SEQ ID NO: 4. In other aspects, the second polypeptide comprises, consists essentially of, or consists of SEQ ID NO: 4 and +1, +2, +3, +4, +5, +6, +7, +8, +9, +10, +11, +12, +13, +14, +15, +16, +17, +18, +19, +20, +21, +22, +23, +24, or +25 amino acids. In some aspects, the second polypeptide comprises, consists essentially of, or consists of SEQ ID NO: 4 with no more than 25, 24, 23, 22, 21, 20, 19, 18, 17, 16, 15, 14, 13, 12, 11, 10, 9, 8, 7, 6, 5, 4, 3, 2, or 1 amino acids. In some aspects, the second polypeptide comprises, consists essentially of, or consists of an amino acid sequence at least about 80%, at least about 85%, at least about 90%, at least about 95%, at least about 96%, at least about 97%, at least about 98%, at least about 99%, or about 100% identical to the amino acid sequence set forth in SEQ ID NO: 3.

In some aspects, the fusion protein comprises one or more mutations. In some aspects, the one or more mutations are one or more substitutions of an amino acid of IL2Rα that is glycosylated with an amino acid that is not glycosylated.

In some aspects, the one or more substitutions amino acids of IL2Rα are at amino acid N49, amino acid N68, amino acid T74, amino acid T85, amino acid T197, amino acid T203, amino acid T208, and amino acid T216, or any combination thereof, wherein the amino acid locations correspond to SEQ ID NO: 1.

In some aspects, the one or more substitutions are from asparagine to another amino acid. In some aspects, the one or more substitutions is from asparagine to an amino acid selected from the group consisting of alanine, threonine, serine, arginine, aspartic acid, glutamine, glutamic acid, glycine, histidine, isoleucine, leucine, lysine, methionine, phenylalanine, proline, tryptophan, tyrosine, and valine.

In some aspects, the one or more substitutions are from threonine to another amino acid. In some aspects, the one or more substitutions is from threonine to an amino acid selected from the group consisting of alanine, arginine, asparagine, aspartic acid, cysteine, glutamine, glutamic acid, glycine, histidine, isoleucine, leucine, lysine, methionine, phenylalanine, proline, serine, tryptophan, tyrosine, and valine.

In some aspects, the substitution is amino acid N49 of SEQ ID NO: 1. In some aspects, amino acid N49 of SEQ ID NO: 1 is mutated to an amino acid selected from the group consisting of alanine, threonine, serine, arginine, aspartic acid, glutamine, glutamic acid, glycine, histidine, isoleucine, leucine, lysine, methionine, phenylalanine, proline, tryptophan, tyrosine, and valine.

In some aspects, the substitution is amino acid N68 of SEQ ID NO: 1. In some aspects, amino acid N68 of SEQ ID NO: 1 is mutated to an amino acid selected from the group consisting of alanine, threonine, serine, arginine, aspartic acid, glutamine, glutamic acid, glycine, histidine, isoleucine, leucine, lysine, methionine, phenylalanine, proline, tryptophan, tyrosine, and valine.

In some aspects, the substitution is amino acid T74 of SEQ ID NO: 1. In some aspects, amino acid T74 of SEQ ID NO: 1 is mutated to an amino acid selected from the group consisting of alanine, arginine, asparagine, aspartic acid, cysteine, glutamine, glutamic acid, glycine, histidine, isoleucine, leucine, lysine, methionine, phenylalanine, proline, serine, tryptophan, tyrosine, and valine.

In some aspects, the substitution is amino acid T85 of SEQ ID NO: 1. In some aspects, amino acid T85 of SEQ ID NO: 1 is mutated to an amino acid selected from the group consisting of alanine, arginine, asparagine, aspartic acid, cysteine, glutamine, glutamic acid, glycine, histidine, isoleucine, leucine, lysine, methionine, phenylalanine, proline, serine, tryptophan, tyrosine, and valine.

In some aspects, the substitution is amino acid T197 of SEQ ID NO: 1. In some aspects, amino acid T197 of SEQ ID NO: 1 is mutated to an amino acid selected from the group consisting of alanine, arginine, asparagine, aspartic acid, cysteine, glutamine, glutamic acid, glycine, histidine, isoleucine, leucine, lysine, methionine, phenylalanine, proline, serine, tryptophan, tyrosine, and valine.

In some aspects, the substitution is amino acid T203 of SEQ ID NO: 1. In some aspects, amino acid T203 of SEQ ID NO: 1 is mutated to an amino acid selected from the group consisting of alanine, arginine, asparagine, aspartic acid, cysteine, glutamine, glutamic acid, glycine, histidine, isoleucine, leucine, lysine, methionine, phenylalanine, proline, serine, tryptophan, tyrosine, and valine.

In some aspects, the substitution is amino acid T208 of SEQ ID NO: 1. In some aspects, amino acid T208 of SEQ ID NO: 1 is mutated to an amino acid selected from the group consisting of alanine, arginine, asparagine, aspartic acid, cysteine, glutamine, glutamic acid, glycine, histidine, isoleucine, leucine, lysine, methionine, phenylalanine, proline, serine, tryptophan, tyrosine, and valine.

In some aspects, the substitution is amino acid T216 of SEQ ID NO: 1. In some aspects, amino acid T216 of SEQ ID NO: 1 is mutated to an amino acid selected from the group consisting of alanine, arginine, asparagine, aspartic acid, cysteine, glutamine, glutamic acid, glycine, histidine, isoleucine, leucine, lysine, methionine, phenylalanine, proline, serine, tryptophan, tyrosine, and valine.

In some aspects, the fusion protein comprises one or more mutations. In some aspects, the one or more mutations is one or more substitutions of an amino acid of IL2Rα that allows glycosylation at a nearby amino acid with an amino acid that does not allow glycosylation at the nearby amino acid.

In some aspects, the substitution is at amino acid S50, amino acid S51, amino acid T69, amino acid T70, amino acid C192, or any combination thereof, wherein the amino acid locations correspond to SEQ ID NO: 1.

In some aspects, the substitution is amino acid S50 corresponding to SEQ ID NO: 1. In some aspects, amino acid S50 corresponding to SEQ ID NO: 1 is mutated to proline.

In some aspects, the substitution is amino acid S51 corresponding to SEQ ID NO: 1. In some aspects, amino acid S51 corresponding to SEQ ID NO: 1 is mutated to an amino acid selected from the group consisting of alanine, arginine, asparagine, aspartic acid, cysteine, glutamine, glutamic acid, glycine, histidine, isoleucine, leucine, lysine, methionine, phenylalanine, proline, serine, tryptophan, tyrosine, and valine.

In some aspects, the substitution is amino acid T69 corresponding to SEQ ID NO: 1. In some aspects, amino acid T69 corresponding to SEQ ID NO: 1 is mutated to proline.

In some aspects, the substitution is amino acid T70 corresponding to SEQ ID NO: 1. In some aspects, amino acid T70 corresponding to SEQ ID NO: 1 is mutated to an amino acid selected from the group consisting of alanine, arginine, asparagine, aspartic acid, cysteine, glutamine, glutamic acid, glycine, histidine, isoleucine, leucine, lysine, methionine, phenylalanine, proline, threonine, tryptophan, tyrosine, and valine.

In some aspects, the substitution is amino acid C192 corresponding to SEQ ID NO: 1. In some aspects, amino acid C192 corresponding to SEQ ID NO: 1 is mutated to an amino acid selected from the group consisting of alanine, arginine, asparagine, aspartic acid, glutamine, glutamic acid, glycine, histidine, isoleucine, leucine, lysine, methionine, phenylalanine, proline, serine, threonine, tryptophan, tyrosine, and valine.

### II. C.3. Linkers of the IL2 Fusion Proteins

The fusion protein of the present disclosure can further comprise a linker. In some aspects, the linker can link the first polypeptide to the second polypeptide from N-terminus to C-terminus, e.g., N-IL2-linker-IL2Rα EC-C. In other aspects, the linker can link the second polypeptide to the first polypeptide from N-terminus to C-terminus, e.g., N-IL2Rα EC-linker-IL2-C.

In one aspect, the IL2 fusion protein comprises a linker sequence located between the IL2 polypeptide and the IL2Rα polypeptide. The linker can be of any length and can comprise at least 1, 2, 3, 4, 5, 6, 7, 8, 9, 10, 11, 12, 13, 14, 15, 16, 17, 18, 19, 20, 25, 30, 31, 32, 33, 34, 35, 36, 37, 38, 39, 40, 50, or 60 or more amino acids. In other aspects, a linker useful for the present disclosure has at least one amino acid and less than 100 amino acids, less than 90 amino acids, less than 80 amino acids, less than 70 amino acids, less than 60 amino acids, less than 50 amino acids, less than 40 amino acids, less than 30 amino acids, less than 20 amino acids, less than 19 amino acids, less than 18 amino acids, less than 17 amino acids, less than 16 amino acids, less than 15 amino acids, less than 14 amino acids, less than 13 amino acids, or less than 12 amino acids. In one aspect, the linker sequence comprises glycine amino acid residues. In other instances, the linker sequence comprises a combination of glycine and serine amino acid residues.

In some aspects, the fusion protein comprises a linker fused in frame between the first polypeptide and the second polypeptide. In some aspects, the fusion protein comprises a linker is a glycine/serine linker. Such glycine/serine linkers can comprise any combination of the amino acid residues, including, but not limited to, the peptide GGGS (SEQ ID NO: 15) or GGGGS SEQ ID NO: 16) or repeats of the same, including 1, 2, 3, 4, 5, 6, 7, 8, 9, 10 or more repeats of these given peptides. The glycine/serine linkers disclosed herein comprises an amino acid sequence of (GS)ₙ, (GGS)ₙ, (GGGS)ₙ, (GGGGS)ₙ, or (GGGGS)ₙ, wherein n is an integer of 1, 2, 3, 4, 5, 6, 7, 8, 9, or 10. In one aspect, the linker sequence comprises GGGSGGGSGGGS (SEQ ID NO: 6) (also noted as (Gly₃Ser)₃). In another aspect, the linker sequence comprises GGGSGGGSGGGSGGGS (SEQ ID NO: 17) (also noted as (Gly₃Ser)₄). In other aspects, the linker sequence comprises one of (Gly₃Ser)₅ (GGGSGGGSGGGSGGGSGGGS) (SEQ ID NO: 18), (Gly₃Ser)₆ (GGGSGGGSGGGSGGGSGGGSGGGS) (SEQ ID NO: 19), or (Gly₃Ser)₇ (GGGSGGGSGGGSGGGSGGGSGGGSGGGS) (SEQ ID NO: 20). In other aspects, the linker sequence comprises (Gly₄Ser)₃ (GGGGSGGGGSGGGGS) as set forth in SEQ ID NO: 21. In additional aspects, the linker sequence comprises GGGGSGGGGSGGGGSGGGGS (SEQ ID NO: 22) (also noted as (Gly₄Ser)₄); GGGGSGGGGSGGGGSGGGGSGGGGS SEQ ID NO: 23) (also noted as (Gly₄Ser)₅); (Gly₄Ser)₂ (GGGGSGGGGS) (SEQ ID NO: 24), (Gly₄Ser)₁ (GGGGS) (SEQ ID NO: 25), (Gly₄Ser)₆ (GGGGSGGGGSGGGGSGGGGSGGGGSGGGGS) (SEQ ID NO: 26); (Gly₄Ser)₇ (GGGGSGGGGSGGGGSGGGGSGGGGSGGGGSGGGGS) (SEQ ID NO: 27); or (Gly₄Ser)₅ (GGGGSGGGGSGGGGSGGGGSGGGGS) (SEQ ID NO: 28).

### II. C.4. Heterologous Moieties of the IL2 Fusion Proteins

The fusion protein of the present disclosure can further comprise an additional element, e.g., heterologous moiety. Such elements can aid in the expression of the fusion protein, aid in the secretion of the fusion protein, improve the stability of the fusion protein, allow for more efficient purification of the protein, and/or modulate the activity of the fusion protein. In some aspects, the heterologous moiety is a polypeptide moiety. In other aspects, the heterologous moiety is a non-polypeptide moiety.

In some aspects, the fusion protein comprises a heterologous moiety fused to the first polypeptide. In some aspects, the fusion protein comprises a heterologous moiety fused to the second polypeptide. In some aspects, the fusion protein comprises a heterologous moiety fused to the first polypeptide and the second polypeptide.

In some aspects, the fusion proteins disclosed herein comprise one or more additional heterologous moieties. In some aspects, the heterologous moieties are half-life extending moieties. In some aspects, the heterologous moiety comprises albumin, an immunoglobulin constant region or a portion thereof, an immunoglobulin-binding polypeptide, an immunoglobulin G (IgG), albumin-binding polypeptide (ABP), a PASylation moiety, a HESylation moiety, XTEN, a PEGylation moiety, an Fc region, and any combination thereof.

Examples of heterologous moieties that can be used according to the present disclosure are disclosed in U.S. Publ. Nos. 2019/0359672 A1 and 2019/0300592 A1, each of which is herein incorporated by reference.

### II. C.5. Exemplary IL2 Fusion Proteins

In some aspects, the fusion proteins comprise any one of SEQ ID NO: 29; SEQ ID NO: 5; SEQ ID NO: 30; SEQ ID NO: 31; SEQ ID NO: 32; SEQ ID NO: 33; and SEQ ID NO: 34, or any one of the sequences as recited in Table 3 of U.S. Patent Publication Nos. 2019/0359672 A1 and 2019/0300592 A1.

In some aspects, the fusion proteins comprise any one of the SEQ ID NO: 29, SEQ ID NO: 5, SEQ ID NO: 30, SEQ ID NO: 31, SEQ ID NO: 32, SEQ ID NO: 33, SEQ ID NO: 34 as recited in Table 3 below, and/or the fusion proteins comprise any one of the sequences as recited in Table 3 of U.S. Patent Publication Nos. 2019/0359672 A1 and 2019/0300592 A1.

TABLE 3

| SEQ | | |
|---|---|---|
| ID | | |
| NO: | Construct | Sequence |
| 29 | IL2(21-153)- | APTSSSTKKTQLQLEHLLLDLQMILNGINN |
| | (G3S)3- | YKNPKLTRMLTFKFYMPKKATELKHLQCLE |
| | CD25(22-240) | EELKPLEEVLNLAQSKNFHLRPRDLISNIN |
| | | VIVLELKGSETTFMCEYADETATIVEFLNR |
| | | WITFCQSIISTLTGGGSGGGSGGGSELCDD |
| | | DPPEIPHATFKAMAYKEGTMLNCECKRGFR |
| | | RIKSGSLYMLCTGNSSHSSWDNQCQCTSSA |
| | | TRNTTKQVTPQPEEQKERKTTEMQSPMQPV |
| | | DQASLPGHCREPPPWENEATERIYHFVVGQ |
| | | MVYYQCVQGYRALHRGPAESVCKMTHGKTR |
| | | WTQPQLICTGEMETSQFPGEEKPQASPEGR |
| | | PESETSCLVTTTDFQIQTEMAATMETSIFT |
| | | TEYQ |
| 5 | IL2-CD25(22- | APTSSSTKKTQLQLEHLLLDLQMILNGINN |
| | 212) | YKNPKLTRMLTFKFYMPKKATELKHLQCLE |
| | | EELKPLEEVLNLAQSKNFHLRPRDLISNIN |
| | | VIVLELKGSETTFMCEYADETATIVEFLNR |
| | | WITFCQSIISTLTGGGSGGGSGGGSELCDD |
| | | DPPEIPHATFKAMAYKEGTMLNCECKRGFR |
| | | RIKSGSLYMLCTGNSSHSSWDNQCQCTSSA |
| | | TRNTTKQVTPQPEEQKERKTTEMQSPMQPV |
| | | DQASLPGHCREPPPWENEATERIYHFVVGQ |
| | | MVYYQCVQGYRALHRGPAESVCKMTHGKTR |
| | | WTQPQLICTGEMETSQFPGEEKPQASPEGR |
| | | PESETS |
| 30 | IL2-CD25(22- | APTSSSTKKTQLQLEHLLLDLQMILNGINN |
| | 187) | YKNPKLTRMLTFKFYMPKKATELKHLQCLE |
| | | EELKPLEEVLNLAQSKNFHLRPRDLISNIN |
| | | VIVLELKGSETTFMCEYADETATIVEFLNR |
| | | WITFCQSIISTLTGGGSGGGSGGGSELCDD |
| | | DPPEIPHATFKAMAYKEGTMLNCECKRGFR |
| | | RIKSGSLYMLCTGNSSHSSWDNQCQCTSSA |
| | | TRNTTKQVTPQPEEQKERKTTEMQSPMQPV |
| | | DQASLPGHCREPPPWENEATERIYHFVVGQ |
| | | MVYYQCVQGYRALHRGPAESVCKMTHGKTR |
| | | WTQPQLICTGE |
| 31 | NativeSigPep- | MYRMQLLSCIALSLALVTNSAPTSSSTKKT |
| | IL2-CD25(22- | QLQLEHLLLDLQMILNGINNYKNPKLTRML |
| | 187) | TFKFYMPKKATELKHLQCLEEELKPLEEVL |
| | | NLAQSKNFHLRPRDLISNINVIVLELKGSE |
| | | TTFMCEYADETATIVEFLNRWITFCQSIIS |
| | | TLTGGGSGGGSGGGSELCDDDPPEIPHATF |
| | | KAMAYKEGTMLNCECKRGFRRIKSGSLYML |
| | | CTGNSSHSSWDNQCQCTSSATRNTTKQVTP |
| | | QPEEQKERKTTEMQSPMQPVDQASLPGHCR |
| | | EPPPWENEATERIYHFVVGQMVYYQCVQGY |
| | | RALHRGPAESVCKMTHGKTRWTQPQLICTG |
| | | E |
| 32 | NativeSigPep- | MYRMQLLSCIALSLALVTNSAPTSSSTKKT |
| | HuIL2- | QLQLEHLLLDLQMILNGINNYKNPKLTRML |
| | CD25(22-212)- | TFKFYMPKKATELKHLQCLEEELKPLEEVL |
| | PP | NLAQSKNFHLRPRDLISNINVIVLELKGSE |
| | | TTFMCEYADETATIVEFLNRWITFCQSIIS |
| | | TLTGGGSGGGSGGGSELCDDDPPEIPHATF |
| | | KAMAYKEGTMLNCECKRGFRRIKSGSLYML |
| | | CTGNSSHSSWDNQCQCTSSATRNTTKQVTP |
| | | QPEEQKERKTTEMQSPMQPVDQASLPGHCR |
| | | EPPPWENEATERIYHFVVGQMVYYQCVQGY |
| | | RALHRGPAESVCKMTHGKTRWTQPQLICTG |
| | | EMETSQFPGEEKPQASPEGRPESETSPP |
| 33 | IL2-CD25(22- | APTSSSTKKTQLQLEHLLLDLQMILNGINN |
| | 187) | YKNPKLTRMLTFKFYMPKKATELKHLQCLE |
| | | EELKPLEEVLNLAQSKNFHLRPRDLISNIN |
| | | VIVLELKGSETTFMCEYADETATIVEFLNR |
| | | WITFCQSIISTLTGGGSGGGSGGGSELCDD |
| | | DPPEIPHATFKAMAYKEGTMLNCECKRGFR |
| | | RIKSGSLYMLCTGNSSHSSWDNQCQCTSSA |
| | | TRNTTKQVTPQPEEQKERKTTEMQSPMQPV |
| | | DQASLPGHCREPPPWENEATERIYHFVVGQ |
| | | MVYYQCVQGYRALHRGPAESVCKMTHGKTR |
| | | WTQPQLICTGE |
| 34 | IL2-T23A- | APASSSTKKTQLQLEHLLLDLQMILNGINN |
| | C145S- | YKNPKLTRMLTFKFYMPKKATELKHLQCLE |
| | CD25(22-187) | EELKPLEEVLNLAQSKNFHLRPRDLISNIN |
| | | VIVLELKGSETTFMCEYADETATIVEFLNR |
| | | WITFSQSIISTLTGGGSGGGSGGGSELCDD |
| | | DPPEIPHATFKAMAYKEGTMLNCECKRGFR |
| | | RIKSGSLYMLCTGNSSHSSWDNQCQCTSSA |
| | | TRNTTKQVTPQPEEQKERKTTEMQSPMQPV |
| | | DQASLPGHCREPPPWENEATERIYHFVVGQ |
| | | MVYYQCVQGYRALHRGPAESVCKMTHGKTR |
| | | WTQPQLICTGE |

Biologically active fragments and variants of the mature and unprocessed form of the IL2/IL-Ra EC domain fusion proteins, and the polynucleotide encoding the same, are also provided. Such a functional polypeptide fragment can comprise at least 5, 6, 7, 8, 9, 10, 11, 12, 13, 14, 15, 16, 17, 18, 19, 20, 30, 40, 50, 75, 100, 150, 200, 250, 300, 350, 400, 450, 500 or more continuous amino acids of any one of SEQ ID NOs: SEQ ID NO: 29; SEQ ID NO: 5; SEQ ID NO: 30; SEQ ID NO: 31; SEQ ID NO: 32; SEQ ID NO: 33; and SEQ ID NO: 34. Alternatively, a functional polypeptide variant can comprise at least 75%, 80%, 85%, 90%, 91%, 92%, 93%, 94%, 95%, 96%, 97%, 98%, or 99% sequence identity to the sequence set forth in SEQ ID NOs: SEQ ID NO: 29; SEQ ID NO: 5; SEQ ID NO: 30; SEQ ID NO: 31; SEQ ID NO: 32; SEQ ID NO: 33; and SEQ ID NO: 34.

Active variants and fragments of polynucleotides encoding the IL2/IL-Ra extracellular domain fusion proteins are further provided. Such polynucleotide can comprise at least 100, 200, 300, 400, 500, 600, 700, 800, 1000, 1100, 1200, 1300, 1500, 1800,2000 continuous nucleotides encoding the polypeptides set forth in SEQ ID NOs: SEQ ID NO: 29; SEQ ID NO: 5; SEQ ID NO: 30; SEQ ID NO: 31; SEQ ID NO: 32; SEQ ID NO: 33; and SEQ ID NO: 34 and continue to encode a functional IL2/IL-Ra extracellular domain fusion protein.

In some aspects, the fusion protein of the present disclosure comprises an amino acid sequence at least about 80%, at least about 85%, at least about 90%, at least about 95%, at least about 96%, at least about 97%, at least about 98%, at least about 99%, or about 100% identical to any one of SEQ ID NO: 29; SEQ ID NO: 5; SEQ ID NO: 30; SEQ ID NO: 31; SEQ ID NO: 32; SEQ ID NO: 33; and SEQ ID NO: 34.

The IL2 fusion proteins of the present disclosure can have one or more the following properties/activities: (1) increasing activity of regulatory T cells (Tregs) and/or increasing immune tolerance in low dose IL2 based therapies; (2) increasing immune response and memory in higher dose therapies; (3) increasing IL2 availability when compared to recombinant IL2; and/or (4) increasing persistent IL2 stimulation of IL2R bearing lymphocytes in vivo.

In one aspect, the fusion proteins disclosed herein comprises one or more pharmacokinetic properties selected from the group consisting of an increased half-life, increased Cₘₐₓ, increased area under the concentration-time curve (AUC), increased Cₘᵢₙ, decreased clearance, improved bioavailability, and any combination thereof, compared to the pharmacokinetic property of the polypeptide consisting of IL2 (SEQ ID NO: 2) or SEQ ID NO: 29 (wt IL2-CD25 sequence with the 12mer linker without truncation).

In one aspect, the fusion proteins disclosed herein have an extended half-life compared to IL2 (SEQ ID NO: 2) or SEQ ID NO: 29 (wt IL2-CD25 sequence with the 12mer linker without truncation). In some aspects, the extended half-life is at least about 1.5 fold, at least about 2 fold, at least about 3 fold, at least about 4 fold, at least about 5 fold, at least about 6 fold, at least about 7 fold, at least about 8 fold, at least about 9 fold, at least about 10 fold, at least about 11 fold, at least about 12 fold, at least about 13 fold, at least about 14 fold, at least about 15 fold, at least about 16 fold, at least about 17 fold, at least about 18 fold, at least about 19 fold, at least about 20 fold, at least about 21 fold, or at least about 22 fold compared to the half-life of a polypeptide consisting of IL2 (SEQ ID NO: 2) or SEQ ID NO: 29 (wt IL2-CD25 sequence with the 12mer linker without any truncation).

In some aspects, an increased activity of Tregs that results from the IL2 fusion protein can be assayed in a variety of ways including, for example, (1) an increased representation and number of Tregs in the CD4+ T cell compartment; (2) upregulation of IL2-dependent CD25; (3) increased proliferation as assessed by expression of the proliferative marker Ki67; and (4) an increased fraction of IL2-dependent terminally differentiated Klrg1+Treg subset. Such effects on Tregs can be seen in, for example, in the spleen and/or the inflamed pancreas.

In some aspects, the IL2 fusion protein of the present disclosure increases tolerogenic and immune suppressive Tregs and immunity through increasing T effector/memory responses and, in further aspects, it exhibits improved pharmacokinetics by delivering such responses at (1) lower effective levels of IL2 activity compared to native or recombinant IL2; and/or (2) displays more persistent biological responses than native or recombinant IL2.

In some aspects, the fusion protein has an improved activity over the native or recombinant IL2. For example, the effect of the IL2 fusion protein can increase tolerogenic Tregs at about 2 fold, 5 fold, 10 fold, 20 fold, 30 fold, 40 fold, 50 fold, 60 fold, 70 fold, 80 fold, 90 fold, 100 fold 150 fold, 200 fold or lower level IL2 activity in comparison to native or recombinant IL2. In other aspects, the IL2 fusion protein is more effective than native or recombinant IL2 in inducing persistent augmentation of Tregs and related properties.

It is further recognized that the components of the IL2 fusion proteins disclosed herein can be found any order. In one aspect, the IL2 polypeptide is at the N-terminus and the extracellular domain of IL2Rα is at the C-terminus of the fusion protein.

In some aspects, the fusion protein forms a dimer. In other aspects, the fusion protein is a monomer. Still, in some aspects, the dimer comprises two monomers, and the monomers are associated with each other via covalent bonds. In some aspects, the dimer comprises two monomers, and the monomers are associated via non-covalent bonds.

In some aspect of the disclosure, the fusion protein is more stable than the polypeptide consisting of IL2 (SEQ ID NO: 2) or SEQ ID NO: 29 (wt IL2-CD25 sequence with the 12mer linker without truncation). In some aspects, the fusion protein has one or more properties selected from the group consisting of (i) increased thermodynamic stability compared to a reference protein; (ii) increased TM compared to a reference protein; (iii) increased resistant to degradation compared to a reference protein; (iv) increased resistance to modifications compared to a reference protein; (v) increased stability in vivo compared to a reference protein; and (vi) any combination thereof, wherein the reference protein comprises (i) a first polypeptide comprising an Interleukin-2 (IL2) polypeptide; and (b) a second polypeptide comprising an extracellular domain of an Interleukin-2 Receptor alpha (IL2Rα) polypeptide; and has at least one more glycosylation compared to the fusion protein.

Any of the glycosylation sites of the fusion proteins disclosed herein can be removed by other mechanisms. In some aspects, the fusion protein is deglycosylated enzymatically or chemically. In some aspects, the fusion protein is deglycosylated by alkali, hydrazinolysis, Peptide-N-Glycosidase F (PNGase F), Endo-β-N-acetylglucosaminidase H (Endo H), O-glycosidase, or any combination thereof.

In some aspects, removal of one or more glycosylation sites of the fusion protein is achieved by treatment of the fusion protein with an alkali. In some aspects, the glycans are removed from the glycosylated polypeptides by alkali borohydride treatment. In other aspects, glycosylation sites of the fusion proteins disclosed herein can be removed using alkaline metal carbonates such as sodium carbonate and potassium carbonate. In some aspects, the alkali is used for β-elimination treatment.

In some aspects, removal of one or more glycosylation sites of the fusion protein is achieved by chemical treatment of the fusion protein by means of hydrazinolysis. In one aspect, glycosylations are released from a fusion protein disclosed herein by subjecting the fusion protein to hydrazinolysis, and the released sugar chain is subjected to fluorescence labeling with 2-aminopyridine. See Hase et al. J. Biochem. 95:197 (1984). In some aspects, hydrazinolysis is carried out using an instrument supplied by Oxford GlycoSystems (the GlycoPrep 1000).

In another aspect, removal of one or more glycosylation sites of the fusion protein is achieved by subjecting the fusion protein to trifluoromethanesulfonic acid (TFMS).

In some aspects, removal of one or more glycosylation sites of the fusion protein is achieved by treatment of the fusion protein with an enzyme. In some aspects, the enzyme is a glycosidase. In some aspects, removal of one or more glycosylation sites of the fusion protein is achieved using Peptide-N-Glycosidase F (PNGase F). The concentration of PNGase F can vary and is to be determined empirically. In some aspects, the glycosidase is PNGase F. PNGase F is a commercially available enzyme (e.g., New England Biolabs, Ipswich MA, Cat. #P0704 or #P0710). In some aspects, the PNGase F is a fusion protein. For example, the PNGase F can be PNGase F tagged with a chitin binding domain (CBD) or a PNGase F-SNAP fusion protein. In some aspects, the glycosidase is lyophilized. In some aspects, the glycosidase is a lyophilized PNGase F. In some aspects, the glycosidase is substantially free of animal-derived reagents.

In some aspects, removal of one or more glycosylation sites of the fusion protein is achieved by treatment of the fusion protein with Endo-β-N-acetylglucosaminidase H (Endo H). Endo-H is a glycohydrolase that is secreted by Streptomyces plicatus and a few other Streptomyces species (Tarentino et al., 1976). It cleaves the β-1, 4-glycosidic bond of the N-acetyl glucosamine core of oligosaccharides and leaves one N-acetylchitobiose attached to the asparagine residue of the glycoprotein (Trimble et al., 1978; Muramatsu 1971). The Endo H gene of S. plicatus is 939 bp (GenBank accession AAA26738.1) encodes a 28.9-kDa protein. Endo H from S. plicatus was recently expressed in Pichiapastoris and deglycosylated activity of P. pastoris produced Endo H was demonstrated in vitro, through both co-fermentation and post-fermentation treatments (Wang et al., 2015).

In some aspects, removal of one or more glycosylation sites of the fusion protein is achieved by treatment of the fusion protein with O-glycosidase (New England Biolabs, Ipswich MA). O-glycosides, also called endo-alpha-N-acetylgalactosaminidase, catalyzes the removal of Core 1 and Core 3 O-linked disaccharides from glycoproteins. In some aspects, it releases unsubstituted Ser- and Thr-linked from glycoproteins.

The removal of one or more glycosylation sites of the fusion protein can be achieved after the IL2 protein is produced in a cell culture (e.g., bioreactor), while the IL2 fusion protein is produced in a cell culture, after the fusion protein is harvested, and/or while the fusion protein is being purified. In some aspects, the removal of one or more glycosylation sites can be achieved by adding one or more removal agents during the cell culture while the fusion protein is expressed. In other aspects, the removal of one or more glycosylation sites can be achieved by selecting a particular cell type as a host cell that eliminates glycosylation or has reduced glycosylation (e.g., E. coli or Streptomyces species). In certain aspects, the removal of one or more glycosylation sites is achieved by co-expressing a gene encoding the fusion protein with a gene encoding an enzyme that removes one or more glycosylation.

### II.D. Pharmaceutical Compositions Comprising an IL2 Fusion Protein

In some aspects, the IL2 fusion protein is administered to the subject as part of a pharmaceutical composition comprising the IL2 fusion protein and one or more pharmaceutically acceptable carriers, excipients, and/or stabilizers.

As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the disclosure is formulated to be compatible with its intended route of administration. Examples of routes of administration include parenteral, e.g., intravenous, intradermal, subcutaneous, oral (e.g., inhalation), transdermal (topical), and transmucosal. In addition, it may be desirable to administer a therapeutical dose of the pharmaceutical composition locally to an area in need of treatment. This can be achieved by, for example, local or regional infusion or perfusion during surgery, topical application, injection, catheter, suppository, or implant (for example, implants formed from porous, non-porous, or gelatinous materials, including membranes, such as sialastic membranes or fibers), and the like. In another aspect, the therapeutical dose of the pharmaceutical composition is delivered in a vesicle, such as liposomes (see, e.g., Langer, Science 249:1527-33 (1990) and Treat et al., in Liposomes in the Therapy of Infectious Disease and Cancer, Lopez Berestein and Fidler (eds.), Liss, N.Y., pp. 353-65, 1989).

In yet another aspect, the therapeutical dose of the pharmaceutical composition can be delivered in a controlled release system. In one example, a pump can be used (see, e.g., Langer, Science 249:1527-33 (1990); Sefton, Crit. Rev. Biomed. Eng. 14:201-40 (1987); Buchwald et al., Surgery 88:507-16 (1980); Saudek et al., N Engl. J Med. 321:574-79 (1989)). In another example, polymeric materials can be used (see, e.g., Levy et al., Science 228:190-92 (1985); During et al., Ann. Neural. 25:351-56 (1989); Howard et al., J Neurosurg. 71:105-12 (1989)). Other controlled release systems, such as those discussed by Langer (Science 249:1527-33 (1990)), can also be used.

Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™ PLURONICS™ or polyethylene glycol (PEG).

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances. Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations can be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions includes EDTA. Pharmaceutical carriers also include ethyl alcohol, polyethylene glycol and propylene glycol for water miscible vehicles; and sodium hydroxide, hydrochloric acid, citric acid or lactic acid for pH adjustment.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes, or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor ELS (BASF; Parsippany, NJ), or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride, in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying, which yields a powder of the active ingredient plus any additional desired ingredient from a previously sterile-filtered solution thereof.

For administration by inhalation, the compounds are delivered in the form of an aerosol spray from a pressurized container or dispenser that contains a suitable propellant, e.g., a gas such as carbon dioxide, or a nebulizer. Systemic administration can also be by transmucosal or transdermal means.

For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. The compounds can also be prepared in the form of suppositories (e.g., with conventional suppository bases such as cocoa butter and other glycerides) or retention enemas for rectal delivery.

In one aspect, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated with each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved, and the limitations inherent in the art of compounding such a functional compound for the treatment of individuals. The pharmaceutical compositions can be included in a container, pack, or dispenser together with instructions for administration.

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Sambrook et al., ed. (1989) Molecular Cloning A Laboratory Manual (2nd ed.; Cold Spring Harbor Laboratory Press); Sambrook et al., ed. (1992) Molecular Cloning: A Laboratory Manual, (Cold Springs Harbor Laboratory, NY); D. N. Glover ed., (1985) DNA Cloning, Volumes I and II; Gait, ed. (1984) Oligonucleotide Synthesis; Mullis et al. U.S. Pat. No. 4,683,195; Hames and Higgins, eds. (1984) Nucleic Acid Hybridization; Hames and Higgins, eds. (1984) Transcription And Translation; Freshney (1987) Culture Of Animal Cells (Alan R. Liss, Inc.); Immobilized Cells And Enzymes (IRL Press) (1986); Perbal (1984) A Practical Guide To Molecular Cloning; the treatise, Methods In Enzymology (Academic Press, Inc., N.Y.); Miller and Calos eds. (1987) Gene Transfer Vectors For Mammalian Cells, (Cold Spring Harbor Laboratory); Wu et al., eds., Methods In Enzymology, Vols. 154 and 155; Mayer and Walker, eds. (1987) Immunochemical Methods In Cell And Molecular Biology (Academic Press, London); Weir and Blackwell, eds., (1986) Handbook Of Experimental Immunology, Volumes I-IV; Manipulating the Mouse Embryo, Cold Spring Harbor Laboratory Press, Cold Spring Harbor, N.Y., (1986);); Crooks, Antisense drug Technology: Principles, strategies and applications, 2^{nd} Ed. CRC Press (2007) and in Ausubel et al. (1989) Current Protocols in Molecular Biology (John Wiley and Sons, Baltimore, Md.).

All of the references cited above, as well as all references cited herein and the amino acid or nucleotide sequences (e.g., GenBank numbers and/or Uniprot numbers), are incorporated herein by reference in their entireties.

The following examples are offered by way of illustration and not by way of limitation.

### III. Examples

### III. A. Nonclinical Studies of BMS-986326

### Nonclinical Pharmacology

Biochemical and biophysical characterization of BMS-986326 showed that it exists primarily as a self-blocking homodimer, with a dissociation half-life of approximately 3 days in vitro at 37° C. and an estimated dissociation constant (Kd) of 1 pM. The binding affinities of BMS-986326 to human, cynomolgus monkeys, mouse, and rat CD25 (IL-2Rα) were: 2,410 nM, 2,000 nM, 4,200 nM and 7,500 nM, respectively, and for IL-2Rβγ (as a pre-assembled heterodimer) were: 111 nM, 105 nM, >4,000 nM and >4,000 nM, respectively. Based on these affinity values, monkeys are a suitable species for evaluating BMS-986326 pharmacology, while rodents may exhibit altered pharmacology relative to humans. Due to low levels of active monomer, the in vitro potency of BMS-986326 was decreased by >100-fold compared to recombinant IL-2. BMS-986326 showed an average half-maximal effective concentration (EC50) of 3.4 nM±1.8 in a Kit225 Interferon Regulatory Factor 1 (IRF1)-driven reporter cell line, compared to 0.027 nM±0.014 for recombinant IL-2. In a whole blood assay to measure phosphorylation of STAT5 (pSTAT5) in Treg, a proximal marker of IL-2 signaling, BMS-986326 exhibited an EC50 of 0.23 nM±0.14 in human blood, and 0.078 nM±0.040 in monkey blood. BMS-986326 demonstrated selectivity for the Tregs in the whole blood assay, with near maximal signal detected in Tregs (>90% of Tregs pSTAT5+ at highest concentration of drug), and only partial signal (<50% pSTAT5+ cells at highest concentration of drug at 71 nM) detected in conventional CD4⁺FoxP3⁻ T cells (Tconv), CD8 cells, and NK cells.

Due to significant differences in the affinity of human IL-2 to bind to mouse IL-2Rβγ dimer, as well as to avoid potential anti-drug antibodies (ADA) in chronic efficacy studies, a mouse surrogate (mIL2-CD25) was used to explore the activity of a homologous dimeric fusion protein in mouse disease models. In BALB/c wild-type mice, mIL2-CD25 showed a prolonged pharmacokinetic (PK) relative to fragment crystallizable (Fc) and mIL-2 fusion protein (Fc-mIL2), or daily doses of murine IL2 (mIL2), as well as robust Treg expansion and enhanced Treg selectivity over CD8 cells and NK cells. The mIL2-CD25 molecule was tested in 2 lupus mouse models: NZB/W and MRL/lpr, which display classic manifestations of lupus-like symptoms observed in humans. In both mouse models, mIL2-CD25 treatment in early disease demonstrated robust and dose-dependent pharmacodynamics (“PD”; Treg proliferation and expansion as well as biomarkers for IL-2 signaling on Treg), as well as robust improvements in disease endpoints similar to high-dose steroid treatment: reductions in autoantibodies and kidney damage, and improvements in kidney function. A dose of 0.2 mg/kg was the minimal dose providing maximal efficacy in these studies. In addition, a sub-optimal dose of mIL2-CD25 (0.1 mg/kg) showed additive efficacy when combined with low-dose steroid treatment (0.1 mg/kg prednisolone). Treg percentages (within the CD4⁺ T cell gate) were measured as a primary PD readout in mouse studies. The change in % Treg in the CD4⁺ T cell gate was calculated by subtracting the % Treg in CD4⁺ T cell gate in the vehicle group from the % Treg in the CD4⁺ T cell gate in the treatment group, represented as A Treg %. Across several studies with twice weekly (BIW) dosing, Treg percentages increased above vehicle control levels by Δ12% to Δ18% in the CD4⁺ population, depending on the study and gating definition for Treg, at doses that led to maximal efficacy (0.2 mg/kg). In the NZB/W model, similar efficacy was achieved with BIW and once-every-5-days (Q5D) dosing frequency, despite the fact that Treg levels fell modestly at PK trough for the Q5D studies. Data from the NZB/W disease model was used to determine target PD responses for efficacy to aid in human dose prediction.

The PK, PD, and tolerability of BMS-986326 across a wide range of doses (0.075 mg/kg, 0.25 mg/kg, 0.75 mg/kg and 2.5 mg/kg, corresponding to AUCs of approximately 48, 157, 407, and 1046 μg·h/mL, respectively) was evaluated after a single subcutaneous (SC) injection in monkeys. In this study, there was a clear dose-dependent increase in the frequency, absolute numbers, and percent proliferating Tregs across all doses. There was also an increase in markers indicative of IL-2R signaling, including phosphorylation of STAT5 as well as expression of CD25, measured by flow cytometry analysis, on Tregs at all doses. Despite minimal evidence for changes in pSTAT5 levels or cell surface CD25 expression in Tconv or CD8⁺ T cells at any dose, there was evidence for dose-dependent expansion and proliferation of Tconv and CD8⁺ T cells at the 2 highest doses tested. In addition, poor tolerability was observed in monkeys at the highest dose level of 2.5 mg/kg. At the 0.25 mg/kg well-tolerated, Treg-selective dose, the change from baseline in the peak percentage of Treg in CD4 T cells reached a level (Δ23% above pre-dose at peak, 4.8-fold increase above pre-dose at peak) well above the % Treg levels required for maximal efficacy in mouse models (Δ12%-18% above vehicle controls). In addition, an increase by Δ10% above baseline in % Treg in CD4⁺ T cell gate was sustained at this dose through Day 21 administration. Collectively, these data demonstrate that BMS-986326 elicits robust and prolonged PD responses that are Treg selective at expected efficacious doses.

### Nonclinical Pharmacokinetics

Various in vitro and in vivo studies were conducted to characterize the PK of BMS-986326 in the nonclinical setting. Ligand binding assays for determining serum exposure of BMS-986326 or mIL2-CD25 measured total concentration which include active monomer and inactive dimer forms. Following intravenous (IV) administration, the steady-state volume of distribution (Vss) of BMS-986326 was 0.0954 and 0.0734 L/kg in mice and monkeys, respectively. Total serum clearance (CLTs) of BMS-986326 was 3.25 and 0.769 mL/h/kg and the apparent elimination half-life (T-HALF) after subcutaneous (SC) dosing was 1.18 and 3.30 days in mice and monkeys, respectively. The time of maximum concentration (Tmax) after SC dosing was 7 and 24 hours in mice and monkeys, respectively. The absolute bioavailability following SC dosing was 58% in mice and 46% in monkeys. In vitro studies in human serum (up to 72 hours) and ex-vivo studies in monkey serum samples after SC administration of BMS-986326 (up to 168 hours) did not show signs of linker-cleavage when assessed by the measurement of the liquid-chromatograph tandem mass spectrometry (LC-MS/MS)-based area ratio of IL-2 and CD25 after immuno-capture via IL-2.

Human PK was projected using allometric scaling of monkey PK parameters and was assumed to have similar SC bioavailability as in monkeys. A half-life of 6 days was projected in humans. Human PD responses (including changes in cell count of CD4⁺ Treg, CD8⁺ T cells, and % pSTAT5⁺ Tregs) were projected using the monkey PK/PD model parameters, since BMS-986326 was shown to have comparable binding affinity to monkey and human IL-2Rα and IL-2Rβγ, as well as comparable potency, in vitro, in the monkey and human whole blood assay.

The efficacious dose of BMS-986326 in human was projected using 2 approaches. The first approach was based on maximal preclinical efficacy in mouse models dosed with mIL2-CD25. Since maximal efficacy in the NZB/W mouse model with mIL2-CD25 (0.2 mg/kg BIW) was associated with a Δ14% Treg in CD4⁺ T cells over baseline at trough, a similar PD response at trough was targeted for projecting the human efficacious dose of BMS-986326. The efficacious dose of BMS-986326 in humans to target Δ14% Tregs in CD4⁺ T cells at trough is 6 mg SC, given-once-every-2-weeks (Q2W). At this dose, the projected steady-state maximum serum concentration (Cmax,ss) of BMS-986326 is 1.5 μg/mL, the projected AUC(TAU) is 217 μg·h/mL, and the projected steady-state minimum concentration (Ctrough,ss) is 0.3 μg/mL.

In subsequent studies, a reduced dosing frequency of mIL2-CD25 (0.2 mg/kg Q5D) in the NZB/W mouse model also demonstrated maximal efficacy with a 10-fold lower trough exposure. Therefore, a projected human dose of 6 mg SC given once-a-month (Q1M) which will achieve a 10-fold lower Ctrough,ss (0.03 μg/mL) compared to the 6 mg SC Q2W dose, is predicted to have similar efficacy. The 6 mg Q1M dosing regimen is predicted to achieve a Δ12% Treg in CD4⁺ T cells at peak and a Δ4% Treg in CD4⁺ T cells at trough.

The second approach for projecting the human efficacious dose of BMS-986326 was based on the clinical demonstration of efficacy with recombinant human IL-2 (rhIL-2) in SLE patients. In a clinical study in patients with SLE, rhIL-2 dosed at 1 MIU (Million International Units) SC every other day for 2 weeks, followed by a 2-week treatment free period, resulted in a Treg profile with a peak of Δ5% Tregs in CD4⁺ T cells and a trough of Δ1% Tregs in CD4⁺ T cells. Since this treatment was shown to improve SLE Responder Index-4 (SRI-4) compared to placebo, a similar Treg profile was targeted for estimating the efficacious dose of BMS-986326. Based on the clinical data, the projected efficacious dose of BMS-986326 in humans to achieve a similar Treg profile is 2 mg SC Q1M. At this dose, the projected Cmax,ss of BMS-986326 is 0.4 μg/mL, the projected AUC(TAU) is 71 μg·h/mL, and projected Ctrough,ss is 0.01 μg/mL. Taken together, the efficacious dose range for BMS-986326 in humans is projected to be 2 to 6 mg SC Q1M.

### Nonclinical Toxicology

Rats and monkeys were demonstrated to be relevant toxicologic species based on in vitro receptor subunit binding data and demonstrated pharmacology (selective Treg expansion) in vivo as well as having been used historically for IL-2R agonist molecules. The single- and repeat-dose toxicity of BMS-986326 were characterized in a series of studies in rats for up to 2 weeks and in monkeys up to 12 weeks using selected dosing frequencies. Pivotal good laboratory practice (GLP) repeat-dose studies in both species employed SC administration of BMS-986326 and consisted of two 2-week studies in rats using differing dose frequencies (once weekly [QW] vs twice weekly [2QW]), a 2-week once weekly dosing study in monkeys, and a 12-week (once every 3 week, Q3W) dosing study in monkeys. In addition, single-dose exploratory studies were initially completed in both rats and monkeys to explore the tolerability of BMS-986326, and a single-dose GLP IV toxicity and cardiovascular safety study was also completed in telemeterized monkeys using IV dosing to support IV administration in the first-in-human (FIH) single-ascending dose (SAD) study (IM034001).

In rats, BMS-986326 was noted to be highly immunogenic, with ADA formation occurring in 84% of BMS-986326-treated rats. In general, the presence of ADA was associated with reduced exposure and loss of PD activity, but was not associated with toxicity. In monkeys, ADA generally did not develop after exposure to BMS-986326 in the pivotal toxicity studies and was of low incidence in the exploratory study.

Intended and unintended PD effects occurred in a dose-dependent manner across all doses of each toxicity study and were generally more pronounced in monkeys than in rats. As an IL-2R agonist, BMS-986326 induced maximal phosphorylation of STAT5 in Treg cells (72% to 95%) across all doses in rats and monkeys, increased Treg cells (up to 63× in monkeys) and/or CD25 expression on CD4 Tregs (up to 4.5× in rats), and raised serum levels of IL-10 (up to 2× in rats and 87× in monkeys). In addition to the desired pharmacologic effects on Tregs, BMS-986326 also led to unintended effects including dose-dependent activation of conventional CD4 and CD8 T cell populations and NK cells, with concomitant elevation of serum IL-5, MCP-1, and perforin. At higher doses of BMS-986326 when dosed weekly in monkeys, increases in B cells (1.6×) and other inflammatory cytokines such as IFN-7 (7.9×), IL-1Ra (49×), and IL-6 (5.1×) were also noted. These inflammatory cytokines were not elevated when lower doses of BMS-986326 were given less frequently (Q3W) in the 12-week monkey toxicity study.

All PD effects (intended and unintended) were generally reversible following the post-dose recovery periods. The peak of the pharmacology responses was generally observed at 4-12 days after dosing, and in the case of dosing regimens that were more frequent (ie, twice weekly in rats or weekly in monkeys wherein successive doses were administered at the peak of the PD response), this correlated with a greater incidence and severity of toxicity (see below). In the 12-week monkey study wherein doses were administered less frequently (every 3 weeks), the PD response in both Tregs and non-Tregs just prior to each successive dose (3 weeks post dosing) had decreased from its peak response at 4-12 days post dose, and, as such, there was evidence of target engagement and a proclivity for favorable immunomodulation at all doses, with minimal stimulation of unintended immune response in the majority of animals. Taken together, although both intended and some unintended dose-related pharmacologic effects were observed in the 12-week monkey study with Q3W dosing, the magnitude of increase in Tregs was up to approximately 2 times higher than the increase in conventional T cells across the dose range.

In a single-dose exploratory study in rats given SC doses of 5, 25, 75, or 200 mg/kg, BMS-986326 was not tolerated by male rats at ≥25 mg/kg/day (mean AUC [0-96 h]≥2,080 μg·h/mL) resulting in morbidity or death on Day 5, with clinical pathology changes indicative of hemorrhage, hepatobiliary injury, and functional cholestasis, all consistent with high-dose effects of IL-2. No adverse effects were observed at 5 mg/kg (mean AUC [0-96 h]=501 μg·h/mL).

In a single-dose exploratory tolerability PK/PD study in monkeys at SC doses of 0.075, 0.25, 0.75, or 2.5 mg/kg, doses of up to 0.75 mg/kg (AUC[INF] 407 μg·h/mL) were well tolerated with desired (phosphorylation of STAT5 in T-regs with Treg expansion and increased CD25 expression on Treg and IL-10) and unintended (modest expansion of Tconv and CD8⁺ T cells, increased proinflammatory cytokines including IL-5, MCP-1, perforin, and GM-CSF) changes in PD end points as well as eosinophilia and decreased red cell mass. BMS-986326 was not tolerated at 2.5 mg/kg (AUC[INF] 1040 μg·h/mL) with several monkeys exhibiting liquid feces, decreased activity, abnormal/scaly/red skin, and severe dehydration and a clinical pathology profile indicative of cytokine release, liver toxicity, and renal involvement likely related to diarrhea and dehydration.

In the pivotal single-dose IV toxicity and cardiovascular safety study in monkeys, BMS-986326 was clinically well tolerated at all doses (0.05, 0.15, or 0.5 mg/kg; mean AUC [0-336 h]≤757 μg·h/mL). Unlike recombinant hIL-2, which causes hypotensive effects and capillary leak syndrome (CLS) at high doses, no decreases in blood pressure, CLS-like effects, or other effects on hemodynamic or electrocardiographic parameters were observed at the BMS-986326 doses administered. Noteworthy toxicologic findings at all doses included minimal to marked increases (up to 91.6×) in eosinophils associated with minimal to moderate myeloid hyperplasia of eosinophils in bone marrow, and minimal to mild increase in eosinophilic cellularity in the spleen (correlating with enlarged spleens and increased weights). At the high dose of 0.5 mg/kg, additional noteworthy findings included a transient increase in mean body temperature (up to 1.1° C.) possibly related to elevation of proinflammatory cytokines, a transient decrease in corrected QT interval and minimally increased heart rate (4% relative to baseline), and a clinical pathology profile broadly reflecting a transient, low-grade inflammatory response. Microscopically, there were dose-dependent multisystemic infiltrates of eosinophils and mononuclear cells in some tissues. All findings were considered non-adverse based on low magnitude and nature of change, lack of tissue damage and inflammatory changes in all tissues/organs examined, and absence of associated functional consequences, and the no-observed-adverse-effect level (NOAEL) following single IV doses in monkeys was considered to be the high dose of 0.5 mg/kg IV (mean AUC [0-336 h]=757 μg·h/mL).

In two separate 2-week toxicity studies in rats with distinct dosing frequencies, BMS-986326 was administered at twice weekly doses of 0.5, 1, or 2.5 mg/kg SC or at once weekly doses of 0.25, 0.5, or 1 mg/kg SC, and was clinically tolerated at all doses (≤2.5 mg/kg, mean AUC [0-336 h]≤600 μg·h/mL). The highest exposures were noted during the first week of each study and diminished considerably during the second week, due to ADA impact. Adverse findings occurred only during the twice weekly study and consisted of capsular fibroplasia and inflammation of the spleen at all doses (≥0.5 mg/kg 2QW, mean AUC ≥189 μg·h/mL on Day 1) and decreases in platelet counts at 2.5 mg/kg (0.8× to 0.4×). Splenic capsular fibroplasia with inflammation was considered adverse at all twice weekly doses due to moderate to marked severity and was also associated with minimal to mild inflammation in the mesentery (adjacent to the stomach and pancreas), which further contributed to the adversity of the finding. As a result, a NOAEL was not identified in the twice weekly dosing study. Other notable nonadverse findings at all doses (≥0.5 mg/kg twice weekly), included increases in eosinophils (3× to 22× controls), a reflection of pharmacologic activity of IL-2 agonists and associated with IL-5 increase; and increased incidence and/or severity of eosinophil infiltration into several organs. Overall, the reduced dose levels and frequency (once weekly) selected for the second 2-week rat study were successful in maintaining target engagement and reducing unintended pharmacology as compared to that demonstrated with 2× weekly dosing. The primary BMS-986326-related effects were mostly minimal in severity at all once weekly doses (0.25, 0.5, or 1 mg/kg) and included increased eosinophils (1.9× to 6.7×); minimal capsular fibroplasia/fibrosis of spleen without inflammation or involvement of adjacent mesentery; and pharmacologically mediated infiltration of eosinophils into a few tissues, none of which were considered adverse due to low magnitude of change and lack of evidence of any compromise in functional integrity of organs involved. The NOAEL in rats following once weekly dosing for 2 weeks was 1 mg/kg (mean AUC 162 μg·h/mL).

In the 2-week toxicity study in monkeys at weekly doses of 0.125, 0.25, or 0.75 mg/kg SC, BMS-986326 was clinically tolerated at ≤0.25 mg/kg, but resulted in adverse clinical signs of toxicity consistent with IL-2R agonism and immunostimulation at 0.75 mg/kg including decreased activity, dehydration, erythema, petechia, and elevated body temperature. At all doses, noteworthy BMS-986326-related toxicologic findings were generally dose related, and primarily involved effects on leukocytes (namely eosinophilia [4× to 40×], with associated tissue inflammation/infiltration); decreased red cell mass (0.9× to 0.6× pretest) and platelets (0.9× to 0.6×); congestion of red pulp in spleen; increased liver weights (16% to 75%), correlating with increased cellularity, sinusoid leukocytosis and Kupffer cell hypertrophy; and minimal to moderate myeloid hyperplasia of bone marrow, likely a regenerative response to eosinophilia. Minimal to mild generally multifocal mixed cell inflammation occurred in multiple tissues and organs (dose related number of ˜16 to 28 tissues affected), including the choroid plexus in the brain and choroid of the eye. Mixed cell inflammation in the eye choroid and choroid plexus of brain at ≥0.25 mg/kg/week and decreased RBC mass at 0.75 mg/kg/week were considered to be adverse due to the severity and nature of the findings, but were considered to be nonadverse at 0.125 mg/kg/week, due to minimal severity and low incidence. At the high dose of 0.75 mg/kg/week, additional notable findings included increased heart rate (25% to 28%), with associated decrease in R-R interval, thought to be secondary to cytokine release and considered non-adverse; minimal to moderate hepatocyte vacuolation of liver; minimal to mild cortical tubule regeneration in kidney, and at recovery sacrifice only, minimal to mild axon degeneration of sciatic nerve in 2 males. Based on adverse inflammation in the eye choroid and choroid plexus of the brain at ≥0.25 mg/kg/week (mean AUC[0-168 h])≥234 μg·h/mL) and accompanied by clinical signs and adverse decreases in red cell mass at 0.75 mg/kg/week (mean AUC[0-168 h]≥601 μg·h/mL), the NOAEL was considered to be 0.125 mg/kg/week (mean AUC[0-168 h]) 132 μg·h/mL).

In the 12-week monkey study, dose range and frequencies were reduced compared to the 2-week study with BMS-986326 administered at 0.0625, 0.125, or 0.25 mg/kg SC Q3W (Days 1, 22, 43, and 64). At all doses, BMS-986326-related findings were nonadverse and included minimal to mild non-adverse increases in eosinophil count (3× to 8× control), increased size and weight of spleen (26 to 65%), and minimal eosinophilic myeloid hyperplasia in bone marrow. Importantly, no eosinophilic or mononuclear cell infiltration into other tissues was noted. Based on the absence of clinical effects and the non-adverse nature of the clinical pathology and pathology findings, the NOAEL was considered to be the highest dose tested of 0.25 mg/kg/dose (mean AUC 306 μg·h/mL).

Dose comparisons between BMS-986326 and rhIL-2 nonclinical studies are challenging considering the differing structure of BMS-986326, the propensity of the majority of BMS-986326 to circulate as an inactive dimer, the vastly different PK of BMS-986326 versus rhIL-2, and widely differing dose regimens across species and studies. Qualitatively, the toxicity profile of BMS-986326 is suggestive of some similarities to rhIL-2, but also has striking differences. Similarities include eosinophilia and leukocyte infiltration into tissues, primarily eosinophilic and sometimes including mononuclear cells. These findings are seen in all the BMS-986326 toxicology studies, and are reported with rhIL-2 in a variety of species, under various dosing paradigms and with variable severity. Eosinophilia, which is also observed clinically with both low- and high-dose rhIL-2 (Proleukin® (aldesleukin), Novartis Pharmaceuticals Canada Inc.), is likely secondary to IL-5 production by ILC2s stimulated by IL-2 (Van Gool et al., Blood 124:3572-6 (2014); Anderson et al., Int Rev Exp Pathol. 34 Pt A:57-77 (1993)), and was not adverse in the BMS-986326 studies. With regard to leukocyte infiltration in tissues, the liver was a primary target organ for rhIL-2 but was not a significant target for BMS-986326 at the doses studied in the pivotal toxicity studies (Anderson et al., Int Rev Exp Pathol. 34 Pt A:57-77 (1993); Harada et al., Int Rev Exp Pathol. 34 Pt A:37-55 (1993)). Liver dysfunction is a frequent side effect of high-dose rhIL-2 in oncology indications, while liver dysfunction has not been a feature in clinical trials of low-dose rhIL-2 in multiple indications including GvHD, type 1 diabetes, alopecia areata, and SLE (Castela et al., JAMA Dermatol. 150:748-51 (2014); He et al., Nat Med. 22:991-93 (2016), Klatmann et al., Nat Rev Immunol. 15:283-94 (2015); Koreth et al., Blood 128:130-37 (2016)). In addition to liver dysfunction, CLS limits therapy in humans under high-dose IL-2 oncology regimens. CLS has not been observed with BMS-986326 in GLP pivotal nonclinical studies.

Overall, the NOAEL doses in the pivotal BMS-986326 studies provide a suitable exposure margin for dose initiation and dose escalations proposed for the single-dose first-in-human (FIH) study in normal healthy participants.

### III.B. Phase 1 Clinical Evaluation of BMS-986326

### General Overview

BMS-986326 is assessed in a Phase 1, randomized, double-blind, placebo-controlled, single ascending doses (SAD) study to evaluate the safety, tolerability, pharmacokinetics (PK), and pharmacodynamics (PD) of single doses of BMS-986326 in healthy adult participants.

The primary objective of the study is to assess the safety and tolerability of single ascending intravenous (IV) and subcutaneous (SC) doses of BMS-986326 in healthy participants.

Secondary objectives of the study include:
determining the single-dose PK of IV and SC BMS-986326 in healthy participants;
determining the absolute bioavailability of BMS-986326 after SC administration as compared with IV administration;
assessing PD following SC and IV administration of BMS-986326; and
evaluating the potential for immunogenicity following SC and IV administration of BMS-986326.

### Target Subject Population

Healthy adult participants are eligible for the study. Eligibility criteria for the study are carefully considered to ensure the safety of the study participants and that the results of the study can be used. Enrollment of healthy participants, instead of patients, allows a clear interpretation of the safety results, because there are no confounding factors resulting from changes in disease state, concurrent organ dysfunction, and/or concomitant medications. In addition, the assessment of a new molecular entity in healthy participants avoids risk of a potential exacerbation of disease, if performed in patients.

The inclusion of placebo-treated participants in each cohort of the study facilitates the assessment of any changes from baseline parameters assessed in the study for all study procedures and helps determine whether these changes are associated with administration of BMS-986326 or to study procedures.

### General Design of Study

Participants in the study are randomized into approximately 9 total dose-level cohorts, with 8 cohorts (IV cohorts A1-A5; SC cohorts B1-B3) and 1 optional IV (A6) cohort.

The total duration of the study for each participant is up to 12 weeks, including up to a 28-day screening period, a 21-day in-house observation period at the clinical site and an approximately 34-day outpatient/follow-up period.

Up to approximately 6 cohorts (cohorts A1-A6) receive a single IV infusion of BMS-986326 according to the Procedure for Administering the Investigational Product provided below. Approximately 3 cohorts (cohorts B1-B3) receive a single dose given via SC injection(s) of BMS-986326 according to the Procedure for Administering the Investigational Product provided below. Because this is a first-in-human (FIH) study, the study design allows for safety, tolerability, PK, and PD data to be gathered in a stepwise fashion. SC administration of BMS-986326 occurs after acceptable safety and tolerability are demonstrated in a cohort of participants who have received a similar dose given IV.

A sentinel group of 2 healthy participants is evaluated in all cohorts. This sentinel group is randomized 1:1 to placebo or BMS-986326. The remaining 6 participants within each dose level are randomized 1:5 to placebo or BMS-986326, respectively. At least 120 hours after dosing of the sentinel group, if the safety profile is an acceptable safety profile (based on, at minimum, adverse events (AEs), concomitant medications and procedures, and any other important safety-related clinical observations), the remainder of the cohort are dosed according to the randomized schedule. For IV cohorts, the remaining 6 participants are dosed sequentially with a maximum 2 participants per day. In some aspects, the participants are dosed with an interval of at least 2 hours between participants.

SC dose-level cohorts do not begin until acceptable safety and tolerability is demonstrated in a cohort of participants who received a similar dose given IV, and PD (Treg count and Treg-to-conventional CD4 cells [Tconv] ratio) data are evaluated.

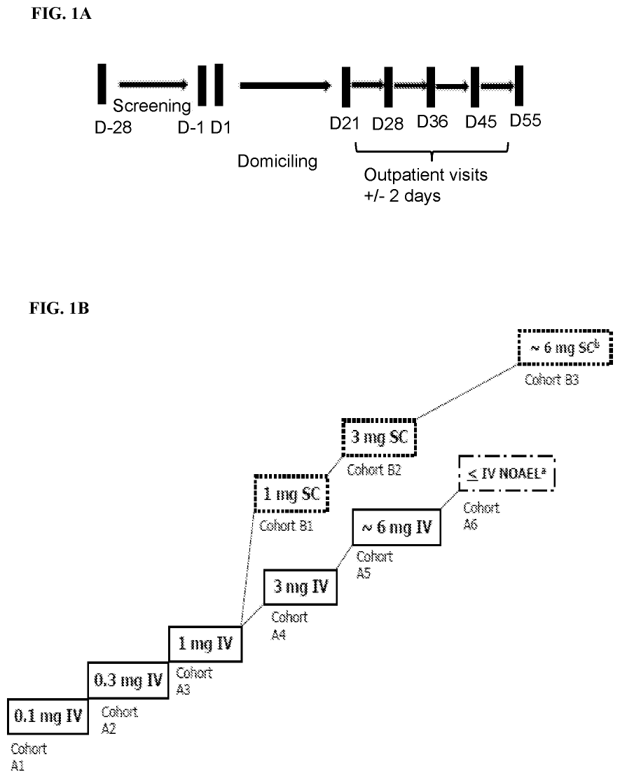

During the course of the study, each participant completes a screening period and treatment period (incudes baseline and outpatient visits), as shown in FIG. 1A. Participants are screened for eligibility. Eligible participants are domiciled at a clinical site from Day −2 or Day −1 until Day 21. The Investigational Product (either placebo or BMS-986326) is administered on Day 1 according to the randomization schedule. Participants are discharged from the clinical site on Day 21 upon satisfactory safety review and completion of the required study procedures. Participants return for outpatient visits on Days 28, 36, 45, and 55 to evaluate the durability of Treg expansion after anticipated peak PD response between Days 10 to 18 and development of potential ADAs. If a participant discontinues from the study early, an early termination visit is performed.

After each cohort completes dosing, safety data including, but not limited to, AEs, physical examinations (PEs), vital signs, 12-lead safety ECGs, injection-site evaluation, clinical laboratory safety test results (including eosinophil counts), concomitant medications/procedures, and PD data (including Treg count and Treg-to-Tconv ratio) are reviewed prior to dose escalation. PK data from completed cohorts are used to predict exposure on an ongoing basis.

The planned dose levels are shown in FIG. 1B. The planned dose levels, including the top dose, can be adjusted depending on emerging PD and PK data from prior cohorts (including bioavailability data from SC cohorts), as described in the Dose-escalating Procedures provided below. When a change to a planned dose-escalation step is required, the maximum dose-escalation step is approximately ≤3-fold the previous dose level. Only IV doses predicted to have a mean exposure area under the serum concentration-time curve extrapolated to infinite time (AUC[INF]) that does not exceed an AUC[0-336 hours (h)] of approximately 757 μg·h/ml are administered. Only SC doses that are predicted to have a mean exposure (AUC[INF]) that does not exceed an AUC(0-504 h)≤306 μg·h/ml are administered.

Escalation to the next dose level may be discontinued if the subject experiences serious adverse event, as described in the Dose Modification/Stopping Criteria provided below. Serious adverse events include any new untoward medical occurrence or worsening of a preexisting medical condition in a clinical investigation participant administered study treatment and that does not necessarily have a causal relationship with this treatment.

### Primary and Secondary Endpoints

The primary endpoints of the present study, which are used to evaluate the primary objective of assessing the safety and tolerability of single ascending IV and SC doses of BMS-986326 in healthy participants, include evaluation of adverse events, clinical laboratory values, vital signs, electrocardiograms, and physical examinations. These assessments are discuss further infra.

The endpoints of the study related to the secondary objective of determining the single-dose pharmacokinetics (“PK”) of IV and SC BMS-986326 in healthy participants include serum PK parameters such as:
Cmax: Maximum observed serum concentration;
Tmax: Time of maximum observed serum concentration;
AUC(0-T): Area under the serum concentration-time curve from time zero to time of last quantifiable concentration;
AUC(INF): Area under the serum concentration-time curve from time zero extrapolated to infinite time;
CLT/F or CLT: Apparent total body clearance or total body clearance for IV;
Vz/F or Vz: Apparent volume of distribution at terminal phase or volume of distribution at terminal phase for IV;
T-HALF: Terminal-phase half-life; and
F: Absolute bioavailability.

The endpoints of the study related to the secondary objective of determining the absolute bioavailability of BMS-986326 after SC administration as compared to IV administration include the following geometric mean ratios of SC (test) to IV (reference): Cmax, AUC(0-T), and AUC(INF) corrected by dose.

The endpoints of the study related to the secondary objective of assessing pharmacodynamics following SC and IV administration of BMS-986326 include measuring the change from baseline in Treg count and Treg-to-Tconv ratio.

The endpoints of the study related to the secondary objective of evaluating the potential for immunogenicity following SC and IV administration of BMS-986326 include measuring the incidence of anti-drug antibodies which may or may not be neutralizing.

### Rationale for Sentinel Dosing

A sentinel dosing strategy is utilized in the study to minimize risk should there be unexpected acute safety events. Two healthy participants (1 active and 1 placebo) are evaluated in all dose cohorts of the study. Each sentinel group is observed for a minimum of 120 hours before the remaining participants of the same cohort are dosed. Based on toxicity studies in monkeys, which showed that peak pharmacology responses were generally observed starting at 4 days after dosing, monitoring of clinical safety for at least 5 days covers potential safety concerns of acute onset such as capillary leak syndrome (CLS) or immune activation (e.g., cytokine release syndrome). The decision to proceed with dosing of the remaining participants in the same cohort as the sentinel participants is made based on the available safety data (e.g., adverse events (AEs), vital signs, physical examinations (PEs), electrocardiograms (ECGs), and clinical laboratory tests).

### Rationale for Dose Range

The dose range selected for the study is expected to provide a range of exposure and pharmacologic activity that provides adequate, stage-appropriate safety data and allows characterization of the PK/PD relationship.

The selection of the starting dose of 0.1 mg IV is based on the available preclinical PK, toxicology, and pharmacology data. PK/PD modeling and simulations were performed to generate predicted human PK and PD profiles from nonclinical data. The actual dose escalation (no more than approximately 3-fold dose-escalation increment) and the actual doses tested are determined by emerging PK, PD, and safety data from the study. This is an FIH study designed to allow for safety, tolerability, PK, and PD data.

Table 4 lists the projected PD changes and exposures resulting from the proposed doses:

TABLE 4
Projected Exposures and Peak Pharmacodynamic Responses at Proposed Human Doses

| | | | | | | | | | |
|---|---|---|---|---|---|---|---|---|---|
| | | | Margin | Margin | Margin | Margin | | | |
| | | | NOAEL | NOAEL | NOAEL | NOAEL | Δ% | | |
| | | AUC | Cmax | AUC | Cmax | AU | Treg | Fold- | Fold- |
| Dose, | Cmax, | (INF), | SDIV | (SD IV | (MD SC | (MD SC | in | change | change |
| mg | μg/mL | μg · h/mL | Monkey)^{a,c} | Monkey)^{a,c} | Monkey)^{b,c} | Monkey)^{b,c} | CD4+ | Tregs | CD8+ |

IV Dosing

| | | | | | | | | | |
|---|---|---|---|---|---|---|---|---|---|
| 0.1 | 0.087 | 7.81 | 140.2 | 96.9 | 28 | 39 | <1 | <2 | <2 |
| 0.3 | 0.258 | 23.2 | 47.3 | 32.6 | 9.0 | 12.6 | 2 | <2 | <2 |
| 1 | 0.864 | 7.77 | 14.1 | 9.7 | 3.0 | 4.2 | 5 | 2-4 | <2 |
| 3 | 2.59 | 232.8 | 4.7 | 3.3 | 1.0 | 1.4 | 12 | 4-8 | <2 |
| 6 | 5.18 | 465.9 | 2.4 | 1.6 | 0.5 | 0.7 | 20 | 7-15 | ~2 |
| 9^{d} | 7.77 | 698.9 | 1.6 | 1.1 | 0.3 | 0.43 | 25 | 10-20 | ~2 |

SC Dosing

| | | | | | | | | | |
|---|---|---|---|---|---|---|---|---|---|
| 1 | 0.21 | 35.7 | 60.0 | 21.0 | 12.0 | 8.4 | 2 | 2-3 | <2 |
| 3 | 0.61 | 106.6 | 20.0 | 7.0 | 4.0 | 2.8 | 7 | 3-5 | <2 |
| 6 | 1.2 | 217 | 10.0 | 3.5 | 2.0 | 1.4 | 12 | 4-8 | <2 |
| 8^{e} | 1.6 | 286.4 | 4.2 | 2.64 | 1.4 | 1.06 | 16 | 5-10 | ~2 |

Abbreviations: AUC = area under the serum concentration-time curve; AUC(INF) = area under the serum concentration-time curve from time zero extrapolated to infinite time; Cmax = maximum observed serum concentration; IV = intravenous; MD = multiple dose; NOAEL = no-observed-adverse-effect-level; Q3W = once every 3 weeks; SC = subcutaneous; SD = subcutaneous; SD = single dose; Treg = regulatory T cell.
^{a}The single-dose IV monkey exposures are used to guide IV dose range in humans.
^{b}Monkey 3-month (Q3W) SC exposures are used to guide SC dose range in humans.
^{c}Safety margins calculated for IV human dose from nonclinical SC NOAEL dose, and vice versa, are adjusted for 46% SC projected bioavaiability of BMS-986326 in humans.
^{d}The optional Cohort A6 will be conducted if less than expected PD effects are observed.
^{e}This dose represents the projected dose, based on current preclinical dose predictions, that is expected to provide a mean AUC(INF) similar to the NOAEL (AUC [0-504 h]) for the 12-week Q3W SC monkey study.

Pharmaceutical Properties and Formulation of the Investigational Product (BMS-986326-01 for Injection, 30 mg/Vial (25 mg/mL))

BMS-986326-01 for injection (30 mg/Vial; 25 mg/mL) has been developed to be used as an IV infusion or SC injection(s) for the Phase 1 clinical study. The drug product is a non-pyrogenic lyophile, which is white to off-white, whole or fragmented cake contained in a 3-cc Type I glass vial, closed with a 13-mm stopper, and sealed with a 13-mm aluminum seal. Each vial of drug product contains the labeled amount of BMS-986326 drug substance, monobasic sodium phosphate, dibasic sodium phosphate, sucrose, pentetic acid, and polysorbate 80, and hydrochloric acid and sodium hydroxide (for pH adjustment), at a pH of 7.0. A 0.31-mL overfill is included in each vial to account for VNS (vial, needle, syringe) holdup. The drug product is reconstituted prior to administration.

Prior to administration, each vial of BMS-986326-01 for injection (30 mg/Vial; 25 mg/mL) is reconstituted with 0.9% sodium chloride injection (normal saline) to a protein concentration of 25 mg/mL. For SC use, the drug product can be administered through an in-line filter as a bolus SC injection, either undiluted at a protein concentration of 25 mg/mL, or diluted with 0.9% sodium chloride injection down to a protein concentration of 0.2 mg/mL. For IV use, the drug product is infused through an in-line filter; the drug product is diluted with 0.9% sodium chloride injection to within a protein concentration range of 0.2 mg/mL to 5 mg/mL prior to infusion.

No incompatibilities have been observed between BMS-986326 injection and Di(2-Ethylhexyl)Phthalate (DEHP)-free polyolefin or PVC (DEHP-plasticized) bags, DEHP-free or PVC (DEHP-plasticized) IV sets, and 0.2 μm polyethersulfone or nylon filters.

The placebo for BMS-986326-01 for injection is commercially available 0.9% sodium chloride injection.

Vials of BMS-986326-01 for injection (30 mg/Vial; 25 mg/mL) are stored refrigerated at 2°-8° C. (36°-46° F.) and protected from light and freezing.

Reconstituted and diluted solutions of BMS-986326-01 for injection may be stored under refrigeration at 2°-8° C. (36°-46° F.) for up to 24 hours, and a maximum of 4 hours of the total 24 hours can be at room temperature of 15°-25° C. (59°-77° F.) with exposure to room light. The maximum 4-hour period under room temperature and room light conditions includes the product administration period.

### Procedure for Administering the Investigational Product

Participants in IV cohorts (A1-A6) receive a single dose administered via IV infusion on Day 1. Participants in SC cohorts (B1-B3) receive a single dose administered via SC injection(s) on Day 1. Planned dose levels for each cohort are included in Table 5 below. As described in the Dose-escalation Procedures provided below, the planned dose levels, including the top dose, may change depending on emerging PD and PK data from prior cohorts, including bioavailability data from SC cohorts. Should a change to the planned dose-escalation step(s) be required, the maximum dose-escalation step will be approximately ≤3-fold the previous dose level.

Each participant receives an SC or IV dose dependent on the dose cohort of BMS-986326. The SC injection(s) is administered slowly and steadily into an abdominal skin fold (except for 5 cm around the umbilicus). Each injection at maximum is a volume of 2 mL. Injection-site reactions are monitored for reactions as described below. BMS-986326 is infused over approximately 30 to 60 minutes. Shorter infusion times may be used for initial-dose cohorts, and longer infusion times may be used for the higher-dose cohorts. Infusion-related reactions (IRR) are monitored.

TABLE 5

| | | | |
|---|---|---|---|
| | | Dosage | |
| | Unit Dose | Formulation | |
| Study | Strength(s)/Dosage | Frequency of | Route of |
| Treatment | Level(s)^{a} | Administration | Administration |

Cohort A1

| | | | | |
|---|---|---|---|---|
| BMS-986326 | 0.1 | mg | Infusion | IV |
| | | | Once | |

Cohort A2

| | | | | |
|---|---|---|---|---|
| BMS-986326 | 0.3 | mg | Infusion | IV |
| | | | Once | |

Cohort A3

| | | | | |
|---|---|---|---|---|
| BMS-986326 | 1 | mg | Infusion | IV |
| | | | Once | |

Cohort A4

| | | | | |
|---|---|---|---|---|
| BMS-986326 | 3 | mg | Infusion | IV |
| | | | Once | |

Cohort A5

| | | | |
|---|---|---|---|
| BMS-986326 | Approximately 6 mg | Infusion | IV |
| | | Once | |

Cohort A6 (Optional)

| | | | |
|---|---|---|---|
| BMS-986326 | Maximum IV dose^{b} | Infusion | IV |
| | | Once | |

Cohort B1

| | | | | |
|---|---|---|---|---|
| BMS-986326 | 1 | mg | Injection(s) | SC |

Once

Cohort B2

| | | | | |
|---|---|---|---|---|
| BMS-986326 | 3 | mg | Injection(s) | SC |

Once

Cohort B3

| | | | |
|---|---|---|---|
| BMS-986326 | Approximately 6 mg^{c} | Injection(s) | SC |
| | | Once | |

Abbreviations: AUC(0-T) = area under the serum concentration-time curve from time zero to the time of the last quantifiable concentration; AUC(INF) = area under the concentration-time curve from time zero extrapolated to infinite time; IV = intravenous; NOAEL = no-observed-adverse-effect-level; PD = pharmacodynamics; PK = pharmacokinetic; Q3W = once every 3 weeks; SC = subcutaneous.
^{a}These are proposed doses; however, the actual dose may change depending on safety, PK, and PD data.
^{b}The maximum IV dose will be a dose that is anticipated to provide a mean exposure (AUC[INF]) not greater than the NOAEL (AUC[0-336 h] ≤ 757 μg · h/mL) for the IV single-dose monkey toxicology study.
^{c}A dose higher than 6 mg SC may be tested, up to a dose that is anticipated to provide a mean exposure (AUC[INF]) that will not exceed the NOAEL (AUC[0-504 h] ≤ 306 μg · h/mL) for the 12-week Q3W SC monkey toxicology study.

### Dose-Escalation Procedure: IV Cohorts A1-A6

Dose-escalation decisions are informed by safety, tolerability, and PD (Treg count and Tregto-Tconv ratio). Assessment of safety data reviewed prior to each dose escalation include AEs, PEs, vital signs, 12-lead safety ECGs, clinical laboratory tests, and concomitant medications/procedures. A minimum of 21 days of safety data from the preceding dose-level cohort are reviewed prior to escalation to the next dose-level cohort. Administration at the next dose level does not begin until the safety and tolerability of the preceding (IV or SC) dose-level cohort are evaluated and deemed acceptable.

In addition to safety and tolerability, PD data (Treg count and Treg-to-Tconv ratio) are reviewed after each IV dose-level cohort completes dosing and are used to inform dose escalation decisions. Higher IV dose levels are not explored if emerging PD data indicate that there is not only a plateauing of the PD response (i.e., peak Treg fold increase) within 3 consecutive IV dose-level cohorts but also approximately equal peak fold increases (at least 2-fold) in Treg and Tconv cells, suggestive of loss of selectivity. Safety and PD data from at least 6 out of the 8 evaluable participants within the cohort are required for safety review before dose escalation, provided any discontinuations are not suspected of being related to BMS-986326. For the purposes of dose escalation, an evaluable participant is defined as a participant who has received one dose of the Investigational Product (BMS-986326 or placebo).

PK data from earlier cohorts are used to predict the mean exposure on an ongoing basis as it becomes available. PK data from prior cohorts, including Cohort A5, are used for dose escalation decision making to move from Cohort A5 to optional Cohort A6, in addition to safety and PD data.

Planned dose levels may be modified or eliminated based on data obtained from prior cohorts. The maximum IV dose explored is a dose that is anticipated to provide a mean exposure AUC(INF) that does not exceed the NOAEL exposure (AUC[0-336 h]≤757 μg·h/mL) in any individual participant. Should a change to the planned dose-escalation step(s) be required, the maximum dose-escalation step is approximately ≤3-fold the previous dose level.

### Dose-Escalation Procedure for SC Cohorts B1-B3

SC administration of BMS-986326 to participants in the first SC dose-level cohort (1 mg) initiates after 21 days of safety data from the 1-mg IV dose-level cohort are reviewed. Dose escalation to the next SC dose-level cohort occurs after review of safety, tolerability, and PD (Treg count and Treg-to-Tconv ratio) data in both the preceding SC dose-level cohort (lower dose) and the preceding IV dose-level cohort (similar dose). Assessment of safety data reviewed prior to each dose escalation includes AEs, PEs, vital signs, 12-lead safety ECGs, clinical laboratory tests, and concomitant medications/procedures. A minimum of 21 days of safety data from both the preceding dose-level cohorts are reviewed prior to dose escalation.

Safety data from at least 6 out of the 8 evaluable participants within each SC cohort are reviewed before dose escalation, provided any discontinuations are not suspected of being related to BMS-986326. For the purposes of dose escalation, an evaluable participant is defined as a participant who has received one dose of the Investigational Product (BMS-986326 or placebo).

PK data from earlier cohorts are used to predict the mean exposure on an ongoing basis as it become available. Only SC doses that are predicted not to exceed a steady-state exposure (AUC[0-INF] of approximately 306 μg·hr/mL) in any individual participant are administered.

Planned dose levels may be modified or eliminated based on data obtained from prior cohorts. Should a change to the planned dose-escalation step(s) be required, the maximum dose-escalation step is approximately ≤3-fold the previous dose level.

### Dose Modification/Stopping Criteria

Escalation to the next dose level may not continue as planned if any of the flowing conditions from the preceding cohort are met:
a. A serious AE (SAE) occurs in one or more BMS-986326-treated participants and is considered to be related to BMS-986326;
b. Two or more BMS-986326-treated participants experience a severe AE that is considered to be related to BMS-986326;
c. Occurrence of severe eosinophilia that is considered to be related to study drug administration in two participants in the same cohort. Severe eosinophilia is defined as:
   i. Eosinophilia: >5000 cells/L that persists for more than 5 days;
   ii. Symptomatic eosinophilia (eosinophilia ≥1500 cells/μL): eosinophilia associated with dermatitis, mucositis, or >2-fold elevations in alanine aminotransferase (ALT)/aspartate aminotransferase (AST);
d. Emerging PD data indicate plateauing of the PD response (i.e., peak Treg fold increase) within 3 consecutive IV dose-level cohorts and emerging PD data indicate approximately equal peak fold increases (at least 2-fold) in Treg and Tconv cells;
e. Any other event that is deemed by the investigator or Sponsor's Medical Monitor to pose an unacceptable risk to participants as a result of dose escalation.

If any of the above are met, a review of available safety, PD (Treg count and Tregto-Tconv ratio), and exposure data is performed. Once an evaluation of the data occurs, the following may occur:
a. Dose escalation may continue as originally planned. This is only permissible if the AE(s) that met stopping criteria is judged, after review, as not considered to be related to BMS-986326;
b. The planned dose escalation may be modified to include repetition of the dose level at which the AE(s) that met stopping criteria had occurred, based on the results of the safety and tolerability review or if further characterization of a safety signal is appropriate. This is only permissible if the AE(s) that met stopping criteria is judged, after review, as not considered to be related to BMS-986326;
   i. An intermediate-dose (lower than the dose that met stopping criteria) cohort may be added;
c. Dose escalation may be stopped.

### Protocols for Assessing Safety and Tolerability

At screening, the safety assessments include a complete physical examination, which includes evaluation of general appearance and vital signs as well as eyes, ears, nose, mouth, throat, neck, respiratory, cardiovascular, respiratory, gastrointestinal/abdomen, lymphatic, musculoskeletal, skin, and neurologic exams. The screening assessments further include continuous holter monitoring, a 12-lead ECG, and a review of prior and concomitant medication use. The vital sign monitoring includes body temperature, respiratory rate, blood pressure and heart rate.

During the course of the study, additional physical examinations are performed at various different time-points as a part of the safety assessment. These subsequent physical examinations are targeted and include examination of the head, ears, eyes, neck, and throat; cardiovascular, neurologic, and respiratory systems; abdomen; skin (including injection site assessment); and extremities.

During the course of the study, further safety and tolerability assessments that are performed at various different time-points also include vital sign monitoring; electrocardiograms; continuous holter monitoring; tuberculosis tests; injection site monitoring; COVID-19 screening; and Clinical Safety Laboratory Assessments, which include clinical chemistry, coagulation, and urinalysis.

Additionally, during the course of the study adverse events and serious adverse event assessment are used to evaluate safety. Adverse events generally include any new untoward medical occurrence or worsening of a preexisting medical condition in a clinical investigation participant administered study treatment and that does not necessarily have a causal relationship with this treatment. This assessment is based in part on the laboratory results that are obtained during the course of the study as well as the results of the other aforementioned safety assessments, which assessments are performed throughout the course of the study. Serious adverse events are generally defined as any untoward medical occurrence that, at any dose, either results in death or is life-threatening. The intensity and causality of all adverse events and serious adverse events are evaluated should any occur.

### Protocols for Assessing Pharmacokinetics (PK) and Immunogenicity of BMS-986326

Separate serum samples are collected for PK and anti-drug antibody (ADA) assessments and include a predose serum sample taken up to one hour prior to BMS-986326 on day 1, an end of infusion (EOI) serum sample taken on day 1, and additional serum samples taken throughout the study (e.g., during domicile on days 2-21 and during outpatient visits on days 28, 36, 45, and 55).

Eligible participants are domiciled at a clinical site from Day −2 or Day −1 until Day 21. The Investigational Product (either placebo or BMS-986326) is administered on Day 1 according to the randomization schedule. Participants are discharged from the clinical site on Day 21 upon satisfactory safety review and completion of the required study procedures. Participants return for outpatient visits on Days 28, 36, 45, and 55

Pharmacokinetics of BMS-986326 is derived from serum concentration versus time. The PK parameters that are assessed include Cmax; Tmax; AUC(0-T); AUC(INF); CLT/F or CLT; Vz/F or Vz; T-HALF; and F.

Individual participant PK parameter values are derived by noncompartmental methods by a validated PK analysis program. Actual times are used for the analyses.

The serum samples are analyzed for BMS-986326 by a validated ligand-binding assay that measures total drug, which includes levels of both the dimer and the monomer. PK samples collected from a participant who received placebo are not analyzed. In addition, serum samples are archived for potential monomer analysis. The monomer levels can be measured using a nonvalidated exploratory ligand-binding assay.

Immunogenicity samples are analyzed for anti-BMS-986326 antibodies by validated immunogenicity assay. A predose sample prior to dosing is collected on Day 1; subsequent samplings can be done on Days 15, 28, and 55. Samples that are confirmed positive are titered and banked for the possible contigent future analysis of neutralizing antibodies to endogenous IL-2 using a validated assay.

### Protocols for Assessing Pharmacodynamics (PD) of BMS-986326

Blood samples are collected and measured by flow cytometry for quantitating immune cells such as Treg, Tconv, follicular helper T cells (Tfh), B cells, and NK cells by surface markers that may include, but not limited to, cell lineage and activation markers CD3, CD4, CD8, CD14, CD25, CD39, CD45, CD45RA, CD56, CD127, Foxp3, Helios, CXCR5, CCR7, and Ki67. Blood samples are also collected and measured by flow cytometry for determining BMS-986326 engagement to Treg, Tconv, CD8 T cells, and NK cells identified by pSTAT5.

Ex vivo Treg suppression assay is performed in selected SC dosing cohorts. Blood samples are collected and Treg cells tested for ex vivo of suppressive activity on activated Tconv cells in assays that may include, but not limited to, measurement of T cell proliferation and cytokine secretion.

Preliminary Results from BMS-986326 Cohort A4 (3 mg Single Intravenous Infusion)

Treg was increased from baseline levels in subjects receiving BMS-986326 compared to subjects receiving a placebo.

### III. C. mIL-2/CD25 Fusion Protein Induces Treg Expansion and Immune Suppression in Preclinical Models of Systemic Lupus Erythematosus

### General Overview

Tregs have a well-established role in suppressing immune response and controlling autoimmunity (Bluestone et al., J Clin Invest. 125: 2250-60 (2015); Dominguez-Villar et al., Nat Immunol. 19: 665-73 (2018)). As such, Tregs are critically responsible for the induction and maintenance of self-tolerance. Dysregulation of Treg function has been implicated in numerous autoimmune conditions (Castela et al., JAMA Dermatol. 150: 748-51 (2014); Koreth et al., N Engl J Med. 365: 2055-66 (2011); Saadoun et al., N Engl J Med. 365: 2067-77 (2011)). Promotion of a tolerance-inducing state is a key goal of next generation immunology therapies that target drug-free remission, and induction and activation of Tregs represent an attractive target toward this goal.

IL-2 was initially discovered as a potent T cell growth factor (Gillis et al., J Exp Med. 146: 468-82 (1977)), and many studies have focused on its role in promoting pro-inflammatory immune responses. For example, high-dose IL-2 (typically 500,000 U/kg, repeatedly) is an approved therapy for treating cancer patients to boost T cell and NK cell function; however, the response rates are typically low and the therapy is also accompanied by severe toxicity (Fraenkel et al., J Immunother. 25: 373-8 (2002)). Additional roles of IL-2 were discovered due to the observation of rapid autoimmunity rather than impaired immune responses in mice deficient in IL-2 or IL-2R (Sadlack et al., Cell 75: 253-61 (1993); Suzuki et al., Science 268: 1472-76 (1995); Willerford et al., Immunity 3: 521-30 (1995)). This phenotype results from the loss of the essential role IL-2 plays in Treg development and homeostasis (Cheng et al., J Immunol. 109: 1567-75 (2013); Fontenot et al., Nat Immunol. 6: 1142-51 (2005); Yao et al., Blood 109: 4368-75 (2007)). Consistent with mouse data, mutations in the human IL-2 signaling pathway have been found to be associated with autoimmune diseases; autoimmune risk variants in the IL-2, IL-2RA, and IL-2RB loci have been identified through the Genome-wide Association Study (Abbas et al., Sci Immunol. 3 (2018)). Systemic lupus erythematosus (SLE), specifically, has been identified as an autoimmune disease associated with Treg dysfunction, that has been attributed to an IL-2 deficient state (von Spee-Mayer et al., Ann Rheum Dis. 75: 1407-15 (2016)).

Pre-clinically, low IL-2R signaling has been shown to selectively promote key activities of Tregs, but not T effector (Teff) cells. Treatment of mice with low levels of IL-2 prevented development of diabetes in non-obese diabetic (NOD) mice (Grinberg-Bleyer et al., J Exp Med. 207: 1871-8 (2010); Tang et al., Immunity 28: 687-97 (2008); Yu et al., Immunity 30: 204-17 (2009)). Several small clinical trials with low-dose IL-2 were reported with encouraging results in SLE (He et al., Nat Med. 22: 991-3 (2016); Klatzmann et al., Nat Rev Immunol 15: 283-94 (2015)). However, the recombinant IL-2 treatment requires daily injections. Furthermore, undesirable increases in pro-inflammatory cytokines and in non-Treg cells were also observed.

A fusion protein (FP) of mouse IL-2 (mIL-2) and mouse IL-2Rα (CD25), joined by a noncleavable linker, has shown greater in vivo efficacy than recombinant IL-2 at Treg expansion and control of diabetes in NOD mice (Ward et al., J Immunol. 201: 2579-92 (2018)). In vivo, mIL-2/CD25 is long-lived, persistently and selectively stimulating Tregs (Ward et al.). mIL-2/CD25 fusion protein is demonstrated herein to be efficacious in inducing Treg expansion and inhibiting lupus nephritis in NZB×NZW F1 and MRL/lpr mice based on the levels of proteinuria, the serum autoantibody titers, and the kidney histology scores of inflammation and damage. At its efficacious dose, mIL-2/CD25 does not lead to any increases in pro-inflammatory cytokines or non-Treg cells in BALB/c mice. Taken together, these data support the use of IL-2/CD25 fusion proteins in treating SLE patients.

### Treg CD25 Expression in NZB×NZW Mice

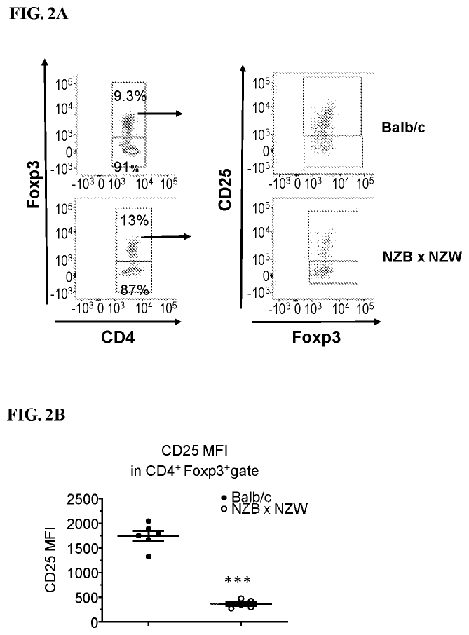

Tregs are dysregulated in SLE patients. It was reported that a high percentage of Tregs (CD4⁺Foxp3⁺) show a lower level of CD25 expression, reflecting an IL-2 deficient state (Humrich et al., Expert Rev Clin Immunol. 12: 1153-60 (2016)). NZB×NZW F1 is a classical model of spontaneous lupus, which develops severe lupus-like phenotypes comparable to that of lupus patients (Xie et al., J Immunol. 192: 4083-92 (2014)). To assess whether NZB×NZW lupus model also captures the Treg abnormality of lower CD25 expression as observed in SLE patients, splenocytes from the NZB×NZW mice (n=5, 26 weeks of age) and the control BALB/c mice (n=6, 9-10 weeks of age) were stained for CD4, FoxP3 and CD25. Representative dot plots are shown in FIG. 2A. The CD4⁺Foxp3⁺ cells represent Tregs. In the representative BALB/c mouse, 73% of Tregs were CD25^{hi}; whereas in the representative NZB×NZW mouse, only 35% of Tregs were CD25^{hi}. The level of CD25 expression was determined by CD25 median fluorescence intensity (MFI) in the Treg gate (CD4⁺Foxp3⁺). As shown in FIG. 2B, the CD25 MFI was 1744.8±98.9 (Mean±SEM) for the BALB/c group, whereas it was only 364.2±34.5 for the NZB×NZW mice. Therefore, the Treg abnormality of low CD25 expression observed in SLE patients is similarly observed in NZB×NZW mice.

Short-Term Treatment of mIL2-CD25 in BALB/c Mice

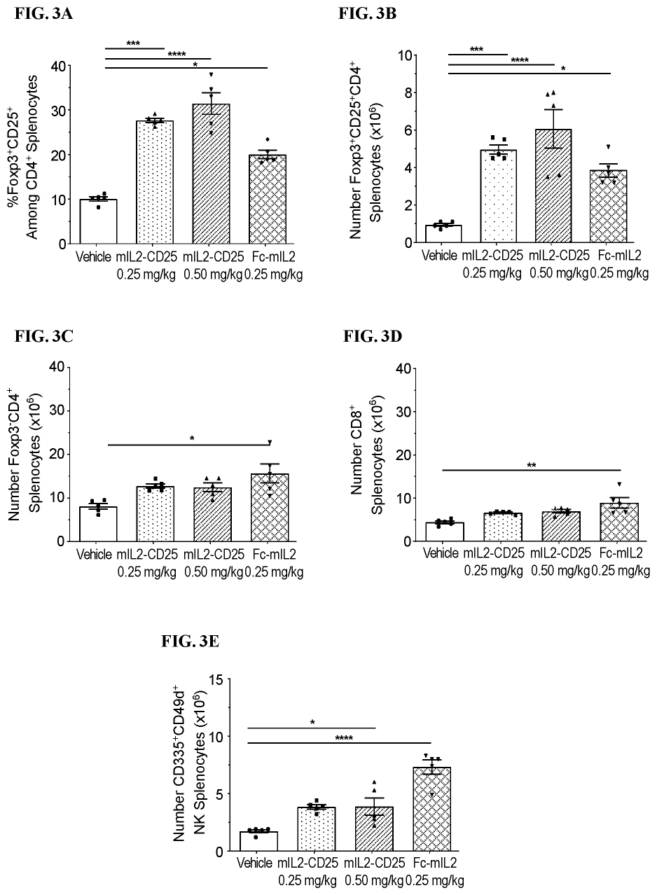

Prior to testing the chronic effect of mIL-2/CD25 in NZB×NZW lupus mice, a short-term study (7-day) was performed in BALB/c mice dosed twice a week for three doses. As a comparator, Fc-mIL2 was dosed every other day for four doses. Spleens were collected and analyzed by flow cytometry, plasma was evaluated for cytokine production. mIL-2/CD25 administration at 0.25 mg/kg and 0.5 mg/kg resulted in a larger increase in the percentage of Tregs in the CD4⁺ gate compared to Fc-mIL2 (FIG. 3A). The percentage of CD8⁺ cells in the single cell gate was reduced due to the increase in Tregs, while neither molecule statistically changed the percentage of NK cells (data not shown). The absolute numbers of various cell types in the spleen were calculated based on the total spleen counts to provide a sensitive measure of potential non-Treg engagement. Similarly, mIL-2/CD25 administration at 0.25 mg/kg and 0.5 mg/kg showed a larger increase in the numbers of Tregs compared to Fc-mIL2 (FIG. 3B). Minimal changes in CD8⁺, CD4⁺Foxp3⁻, and NK cells were observed for mIL2-CD25, whereas Fc-mIL2 significantly increased these populations (FIG. 3C-E). Measurements of circulating cytokines support the Treg selectivity. For mIL-2/CD25 administration at these doses, no cytokines were elevated and only IL-5 was slightly elevated at 0.5 mg/kg dose (data not shown). These results confirmed that mIL2-CD25 provides an improved Treg engagement and selectivity profile relative to Fc-mIL2, with greater Treg increases and no evidence of non-Treg engagement. Based on these results, the dose range of 0.1-0.4 mg/kg twice a week was selected for further testing of mIL-2/CD25 efficacy in animal models of lupus.

mIL-2/CD25 in Early Lupus in NZB×NZW Mice

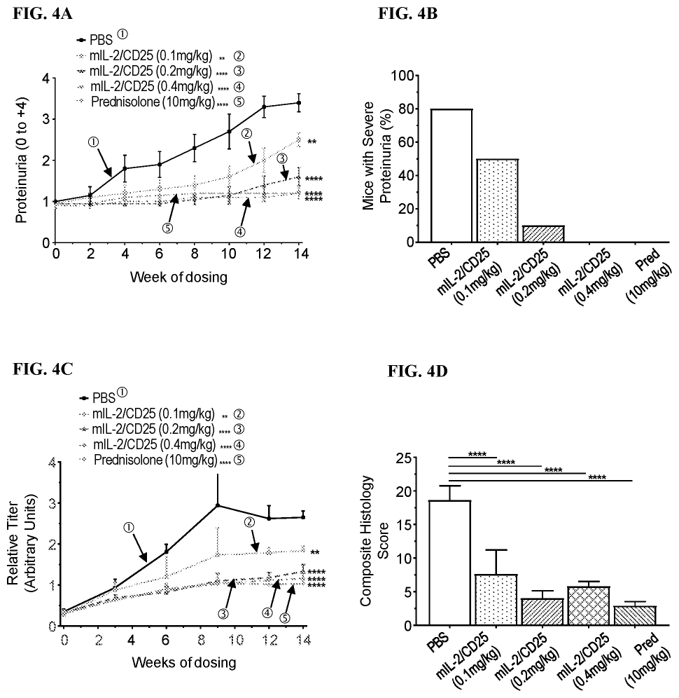

To assess the effect of mIL-2/CD25 in an animal model of SLE, NZB×NZW F1 mice, aged 22-24 weeks, with a proteinuria level at 30 mg/dL (score of 1) were enrolled for the study. Mice were given either s.c. injections of PBS or mIL-2/CD25 twice a week at 0.1, 0.2, and 0.4 mg/kg, or with p.o. dosing of prednisolone at 10 mg/kg three times a week to serve as a positive control (n=10 per group). Serum exposure (AUC and Cmax) of mIL2-CD25, determined after the first dose, increased in a dose-dependent manner between 0.1 and 0.4 mg/kg dose with average terminal half-life of 20.6 hour. PK parameters are presented in Table 6. Over the course of a 14-week experiment, mIL-2/CD25 administration was well tolerated and there was no observed body weight loss (data not shown). Disease progression as judged by proteinuria scores was significantly reduced by mIL-2/CD25 administration in a dose-dependent manner (FIG. 4A). The percentage of animals developing severe proteinuria with a score of 3 or higher was also reduced (FIG. 4B). Mice were also bled and checked for the presence of serum autoantibodies every 2-3 weeks. Anti-dsDNA IgG titers were significantly reduced by mIL-2/CD25 treatment relative to PBS treated mice in a dose-dependent manner, with maximal effect (achieved by both 0.2 and 0.4 mg/kg doses) comparable to that observed in prednisolone controls (FIG. 4C). Microscopic evaluation revealed significant glomerulonephritis in the kidneys of the PBS-treated control mice. This was characterized by varying degrees of hypercellularity and matrix deposition in the glomeruli, protein casts in tubular lumens, and inflammatory cell infiltrates in the interstitial and perivascular tissues. Treatment with mIL-2/CD25 significantly reduced the histological scores on glomerular, tubular and interstitial nephritis (FIG. 4D). In conclusion, mIL-2/CD25 demonstrated efficacy in reducing the level of proteinuria, the dsDNA IgG titers, and the kidney histology scores in a dose-dependent manner in the NZB×NZW lupus model; maximal efficacy was observed at the 0.2 mg/kg and 0.4 mg/kg doses (2×/week), comparable to that observed with high-dose prednisolone.

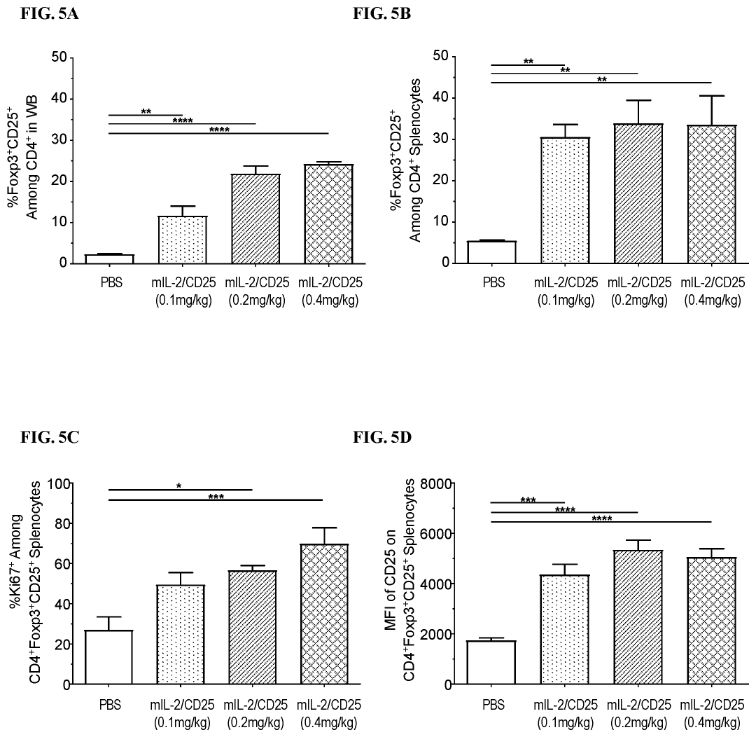

To correlate efficacy with the extent of Treg expansion, blood and spleens (n=4 per group) were collected for flow cytometry after four weeks of dosing (48 hours post 8th dose). mIL-2/CD25 treatment dose dependently increased the percentage of Tregs (CD25⁺Foxp3⁺) in the CD4⁺ gate in both blood and the spleen. In blood, the percentage of Tregs was significantly increased from 2.3±0.1% (Mean±SEM) in the PBS group to 11.7±2.3%, 21.9±1.9%, 24.2±0.5% in the 0.1, 0.2, and 0.4 mg/kg dose groups respectively (FIG. 5A). Similarly, there were increases in the percentages of splenic Tregs at all doses (FIG. 5B). Splenocytes were also stained for Ki67 to demonstrate proliferating status of Tregs. As shown in FIG. 5C, the percentage of Ki67⁺ cells in the Treg gate (CD4⁺CD25⁺Foxp3⁺) was increased in a dose dependent manner from 27.0±6.4% in the PBS group to 49.5±5.9%, 56.6±2.4%, and 69.8±8.0% in the 0.1, 0.2, and 0.4 mg/kg mIL-2/CD25 treatment groups respectively. CD25 expression on Tregs (CD25 MFI), which was lower in NZB×NZW relative to Balb/c mice (FIG. 2B), was also increased in a dose dependent fashion (FIG. 5D) from 1745±96 MFI in the vehicle group to 4361±408, 5340±390, and 5057±335 (±SEM) MFI, in the 0.1, 0.2 and 0.4 mg/kg mIL-2/CD25 treatment groups, respectively. Blood and spleen Tregs were also analyzed after 14 weeks of dosing and results were comparable to those obtained at week 4 (data not shown). In conclusion, mIL-2/CD25 treatment increased both the number of Tregs and the CD25 expression on Tregs, which as a result, inhibited disease progression in NZB×NZW lupus mice.

TABLE 6
Serum exposures of mIL-2/CD25 in NZB × NZW mice.

| Dose | 0.1 mg/kg s.c. | 0.2 mg/kg s.c. | 0.4 mg/kg s.c. |
|---|---|---|---|
| Cmax (nM) | 9.5 | 26.5 | 56.9 |
| Tmax (h) | 24 | 24 | 24 |
| AUC_{(0-80 h)} (nM · h) | 368.9 | 877.2 | 2045.9 |
| T_{1/2} (h) | 24 | 18 | 19 |

Serum levels of mIL-2/CD25 following a 0.1, 0.2 or 0.4 mg/kg s.c. dose of mIL-2/CD25 increased in dose-dependent manner. Pharmacokinetic parameters following 0.1, 0.2 or 0.4 mg/kg s.c. dose were 368.9, 877.2 or 2045.9 nM·h, respectively (AUC0-80 h) and 9.5, 26.5 or 56.9 nM, respectively (Cmax). Tmax was 24 hours. Average terminal half-life was 20.6 hours.

mIL-2/CD25 in Advanced Lupus in NZB×NZW Mice

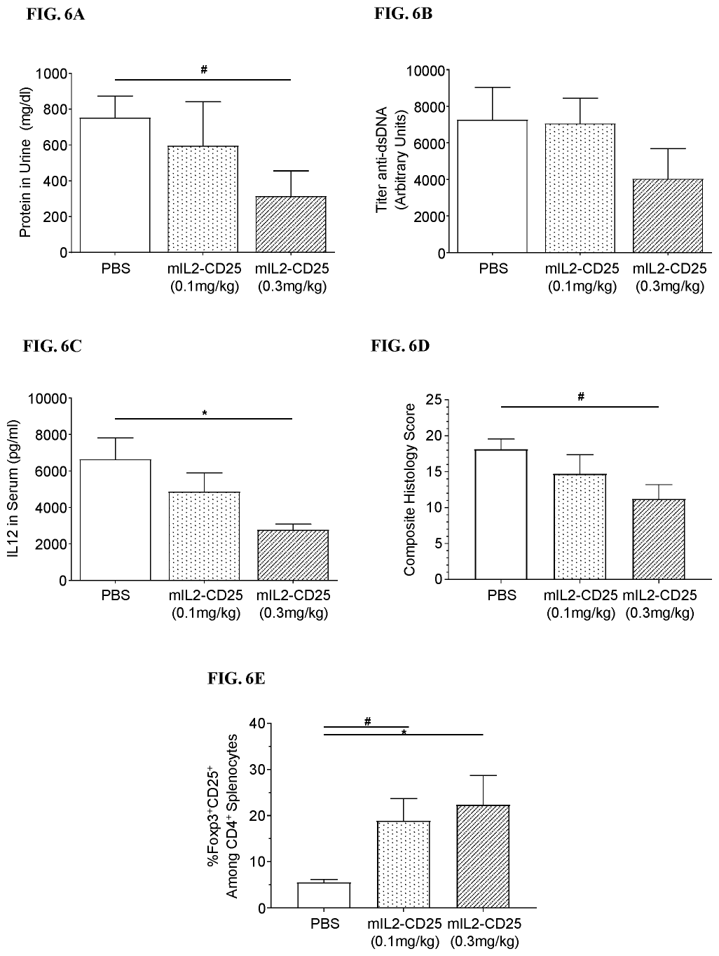

mIL-2/CD25 was further tested for its ability to ameliorate signs of late stage disease, which is a higher bar for efficacy. Mice with advanced proteinuria (≥100 mg/dL; ˜27 weeks of age) were enrolled for a 10-week treatment study. mIL-2/CD25 at 0.3 mg/kg 2×/week showed a significant reduction in the levels of proteinuria (FIG. 6A), the anti-dsDNA IgG titers (FIG. 6B), the levels of plasma IL-12p40 (FIG. 6C), and the kidney histology scores on inflammation and damage (FIG. 6D). At completion of the study, flow cytometry was conducted with splenocytes. The percentage of CD4⁺CD25⁺Foxp3⁺ Tregs in the CD4⁺ gate was increased from 5.5±0.6% (Mean±SEM) in the PBS group to 18.9±4.8% and 22.4±6.3% in the 0.1 mg/kg and 0.3 mg/kg mIL-2/CD25 treatment groups, respectively (FIG. 6E). This data supports the therapeutic potential for IL-2/CD25 in treating more advanced lupus patients.

Effect of mIL-2/CD25 and Prednisone Combination in NZB×NZW Mice

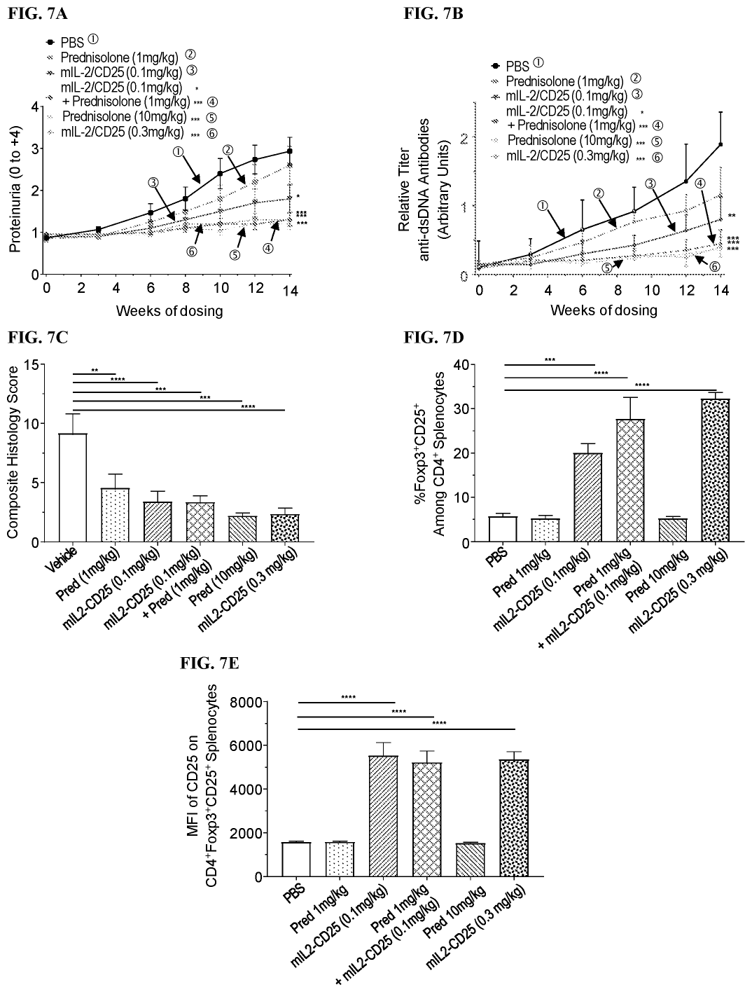

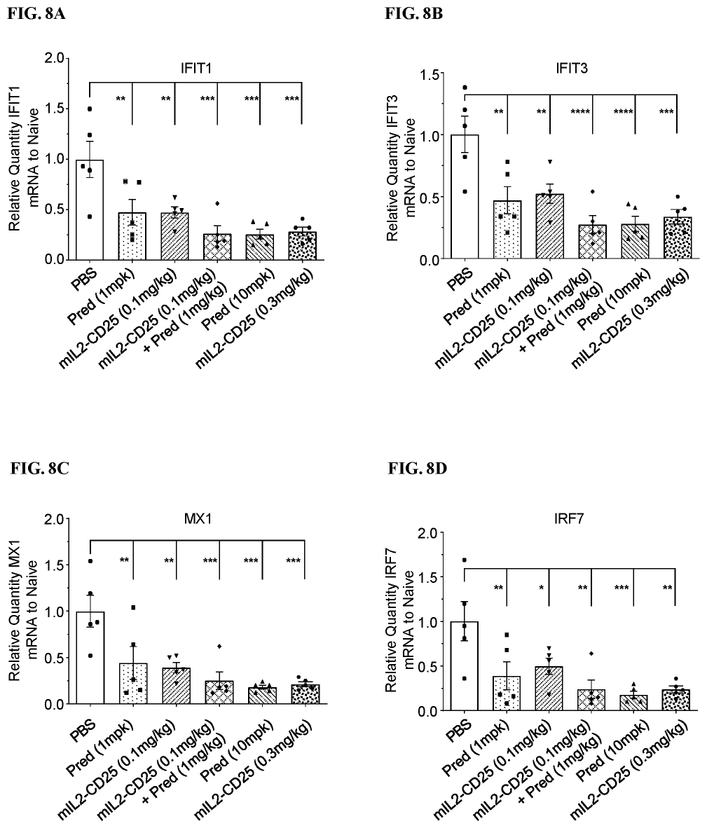

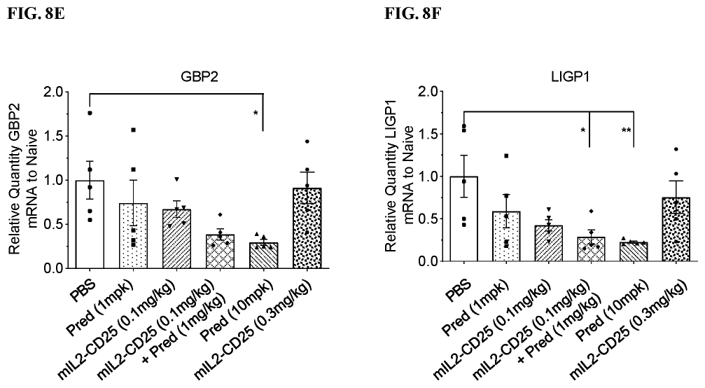

To assess the potential utility of mIL-2/CD25 therapy to reduce dependence on corticosteroids, the current standard of care in lupus, a combination study with partially efficacious doses of mIL-2/CD25 (0.1 mg/kg s.c. 2×/week) and prednisolone (1 mg/kg p.o. 3×/week) was tested in NZB×NZW early lupus mice with proteinuria of 30 mg/dL (21-23 weeks of age). A high dose of prednisolone (10 mg/kg, p.o. 3×/week) and mIL-2/CD25 (0.2 mg/kg s.c. 2×/week) groups were included as maximal efficacy monotherapy controls. As shown in FIG. 7, while monotherapy of either 1 mg/kg prednisolone or 0.1 mg/kg mIL-2/CD25 showed partial protection in reducing the proteinuria levels and the anti-dsDNA titers, the combination of both demonstrated efficacy comparable to high dose prednisolone or mIL-2/CD25 controls, which was superior to either therapy alone (FIGS. 7A and 7B). A superior effect with the combination treatment was also demonstrated in the further reduction of type 1 interferon gene expression in the kidney, such as IFIT1, IFIT3, MX1, IRF7, GBP2 and LIGP1, based on RT-PCR (FIGS. 8A-8F, respectively). The advantage of combination treatment was less evident based on histology scores (FIG. 7C) because a high level of reduction of histology scores was already achieved with 0.1 mg/kg mIL-2/CD25 monotherapy based on the semi-quantitative histology scores.

The effect of mIL-2/CD25 on Tregs was also assessed in the spleen at completion of this study (after 14 weeks of treatment). As expected, prednisolone monotherapies (both 1 and 10 mg/kg groups) did not alter the percentage of Tregs (CD4⁺CD25⁺Foxp3⁺) or CD25 MFI on Tregs. Consistent with previous studies, mIL-2/CD25 monotherapy increased the percentage of Tregs and CD25 MFI on Tregs (FIGS. 7D and 7E). Importantly, the co-treatment of prednisolone did not interfere with the effect of mIL-2/CD25 in increasing the percentage of Tregs or CD25 MFI on Tregs (FIGS. 6D and 6E). These data suggest IL-2/CD25 therapy can be effectively combined with standard of care steroid therapy without losing its efficacy in increasing the numbers of Tregs, and potentially inhibiting the disease progression in SLE patients.

mIL-2/CD25 in MRL/lpr Lupus Model

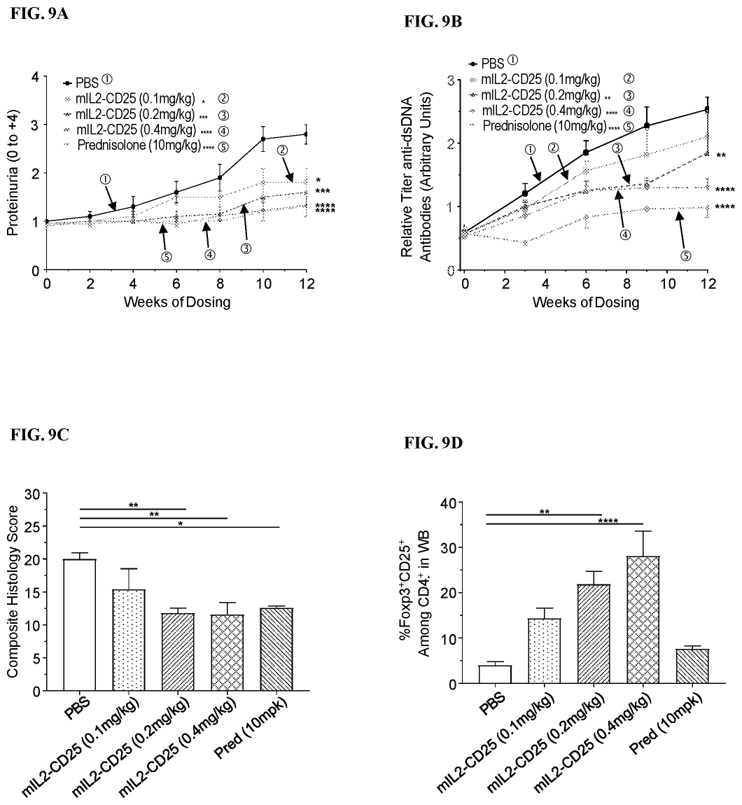

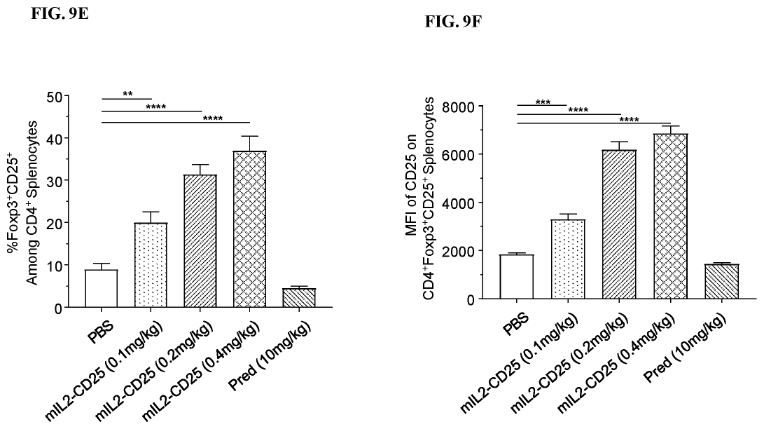

mIL-2/CD25 was also evaluated in another murine model of lupus, MRL/lpr. In this model, unchecked aberrant proliferation of immune cells leads to a spontaneous autoimmune lupus-like syndrome. mIL-2/CD25 dosed s.c. at 0.1, 0.2, or 0.4 mg/kg 2×/week for 12 weeks (n=10 per group) prevented worsening of proteinuria (FIG. 9A), auto-antibody production (FIG. 9B), and kidney inflammation and damage (FIG. 9C). Similar to the data in the NZB×NZW studies, dose range from 0.2 mg/kg to 0.4 mg/kg achieved maximal efficacy in all the above three endpoints. Again, blood and spleens (n=4 from each group) were collected for flow cytometry at completion of the study (12 weeks after first dosing, 48 hours post last dose). mIL-2/CD25 treatment dose dependently increased the percentage of Tregs (CD4⁺CD25⁺Foxp3⁺) in the CD4⁺ gate in both blood and the spleen. In blood, the percent Treg was significantly increased from 4.0±0.8% (Mean±SEM) in the PBS group to 14.3±2.3%, 21.9±6.2%, and 28.1±5.5% in the 0.1, 0.2, and 0.4 mg/kg dose groups respectively (FIG. 9D). Similarly, there were increases in splenic Tregs at all doses (FIG. 9E). As shown in FIG. 9F, the CD25 MFI in the CD4⁺CD25⁺Foxp3⁺ gate was significantly increased from 1842±118 (Mean±SEM) in the PBS group, to 3301±470, 6185±713, and 6863±680 in the 0.1, 0.2, and 0.4 mg/kg mIL-2/CD25 treatment groups respectively. In conclusion, mIL-2/CD25 inhibits disease progression in MRL/lpr mice by increasing the numbers of Tregs and CD25 expression on Tregs.

### Discussion

The management of active SLE is challenging due to the heterogeneous nature of the disease (Franklyn, et al., Nat Rev Rheumatol. 10: 567-71 (2014); Tsokos, N Engl J Med. 365: 2110-21 (2011)). Current therapy of active SLE relies primarily on corticosteroids and immunosuppressants to reduce disease activity. However, these drugs are not completely effective, and thus outcomes are further offset by significant adverse effects, especially treatment-related infections (Goldblatt, et al., Lupus 18: 682-89 (2009); Kang et al., Curr Opin Rheumatol. 15: 528-34 (2003); Bruce, et al., Lupus 25: 699-709 (2016)). Tregs provide broad upstream control of a number of important cell types and pathways in SLE pathogenesis, which differentiates Treg modulation from other SLE clinical pipeline assets. While a prime target of Tregs are effector T cell activities, Treg control of immune response goes beyond Teffector cells, including the potential to influence NK and NK T cells, B cells and macrophages/antigen presenting cells, and to further promote tissue repair (Abbas, et al., Sci Immunol. 3 (2018); Dutcher et al., J Immunother Cancer. 2:26 (2014); Li et al., Front Immunol. 9: 585 (2018); Tiemessen et al., Proc Natl Acad Sci USA 104: 19446-51 (2007); Williams et al., Nature 441: 890-3 (2007)). The use of low-dose IL-2 therapy to promote T regulatory function to suppress inflammation and autoimmunity has drawn attention, based on promising preliminary results in multiple clinical trials including SLE studies (Castela et al., JAMA Dermatol. 150: 748-51 (2014); Saadoun et al., N Engl J Med. 365: 2067-77 (2011), Abbas et al., Sci Immunol. 3 (2018); von Spee-Mayer et al., Ann Rheum Dis. 75: 1407-15 (2016); He et al., Nat Med. 22: 991-3 (2016); Churlaud et al., J Allergy Clin Immunol 142: 1344-6 (2018); Rosenzwajg et al., J Autoimmun. 58: 48-58 (2015); Rosenzwajg et al., Ann Rheum Dis. 78: 209-17 (2019)). The preliminary result of low-dose IL-2 across 11 autoimmune diseases has demonstrated the potential for broad utility with this approach (Rosenzwajg et al., Ann Rheum Dis. 78: 209-17 (2019)).

A long acting IL-2 receptor agonist consisting of IL-2 fused to CD25 with a noncleavable linker has demonstrated improvement over recombinant IL-2 with regard to serum half-life and Treg selectivity in vivo in mouse (Ward et al., J Immunol. 201: 2579-92 (2018)). mIL-2/CD25 fusion protein has a unique mechanism of action (MOA), existing predominantly as a self-blocking, inactive homo-dimeric molecule in solution. Slow release of the active monomer through disassociation and capture by CD25-expressing Tregs leads to cellular activation and proliferation (Ward et al., J Immunol. 201: 2579-92 (2018)). This MOA enables the molecule to achieve a Pharmacokinetic (PK) and Pharmacodynamic (PD) prolongation that has not been achieved with other mechanisms of delivering IL-2 receptor agonism, due in part to the ability of the molecule to avoid target mediated clearance (TMDD) as it circulates in the inactive dimer form. As shown herein, mIL-2/CD25 has prolonged PK (T1/2: 20.6 hrs) and desirable Treg expansion/CD25 upregulation on Tregs in both NZB×NZW and MRL/lpr mice, two common models of lupus and lupus nephritis. Further, analysis of Tregs from the NZB×NZW model demonstrated reduced Treg CD25 levels, similar to the observations from SLE patients, a marker of IL-2 deficiency. The result suggests NZB×NZW model recapitulates elements of the Treg dysfunction observed in human disease. mIL-2/CD25 reversed th apparent IL-2 deficiency in this model leading to increases in both Treg numbers and CD25 expression.

The Treg induction and activation observed with mIL-2/CD25 lead to a significant reduction in disease progression as judged by reduced proteinuria levels, autoantibody titers and kidney histology scores, even when treatment is initiated when NZB×NZW mice have shown signs of advanced disease. The doses tested herein do not activate non-Treg cells or proinflammatory cytokine production. Importantly the dose response relationship suggests that significant and sustained Treg increases are required for maximal efficacy in this model and suggest that robust and sustained, but selective Treg increases will be require for maximal efficacy in human disease.

Corticosteroids are still the mainstay of lupus treatment, especially to treat flare-ups. Corticosteroids are known to inhibit T cell responses; however, Tregs may be less susceptible to steroid treatment than Teffector cells (Prenek, et al., Apoptosis 25: 715-29 (2020)). Demonstrated herein, mIL-2/CD25 treatment can increase Tregs and improve disease even when combined with low dose steroids. The combination treatment results in improvements in most of the efficacy readouts relative to either monotherapy. These results suggest the potential clinical utility of combining the prolonged and selective IL-2R Treg agonism, such as afforded by mIL2/CD25 treatment, with SLE standards of care achieve improved efficacy.

To date, the human IL-2/CD25 fusion protein that has been developed shows prolonged and selective Treg activation in cynomolgus monkeys at defined doses (unpublished results). The human IL-2/CD25 will be tested in clinical trials for evaluation of PK, PD (Treg), safety and tolerability. The hypothesis of selectively targeting SLE patients with IL-2 deficiency in Treg population will be explored in clinic. The potential for this mechanism to offer clinical efficacy as well as steroid sparing and long term remission remains to be seen in future studies.

### Materials and Methods

Reagents:

mIL-2/CD25 is a fusion protein combining murine IL-2 to the murine IL-2 receptor alpha subunit (CD25) with a linker consisting of 12 amino acids between the C terminus of IL-2 and the N terminus of the extracellular region of CD25. mIL-2/CD25 fusion protein forms a noncovalent self-blocking dimer. Biochemical assessments support that the dimer does not bind to the receptor and is therefore protected from target-mediated drug disposition. Slow disassociation yields a low dose of active monomer that results in activation of IL-2R (Ward et al., J Immunol. 201: 2579-92 (2018)). Prednisolone (Sigma-Aldrich, St. Louis, MO) is an anti-inflammatory steroid medication used as a control compound.

The antibody panel for flow cytometry in mouse studies includes: CD4-V500 (clone RM4-5), pSTAT5-AF488 (clone 47/Stat5 pY694) from BD Biosciences, CD8-PerCP-Cy5.5 (clone 53-6.7), CD25-PE (clone PC61.5), Foxp3-ef450 (clone FJK-16s) from ThermoFisher Scientific, and CD335-BV605 (clone 29A1.4), Ki67-APC (clone 16A8) from Biolegend.

### Mice:

Female NZB×NZW F1, female BALB/c, and male MRL/lpr mice were from the Jackson Laboratories (Bar Harbor, ME). All procedures were performed in accordance with protocols approved by the BMS Animal Care and Use Committee.

### Proteinuria Monitoring:

Prior to randomization into treatment groups, mice were evaluated for proteinuria using Albustix (Siemens, Munich, Germany) by inducing mice to urinate on the Albustix strips. Mice with a proteinuria level readout corresponding to trace-30 mg/dL were included in the studies for evaluation of efficacy in early disease. Typical ages of mice enrolled in early disease studies are 21-23 weeks for NZB×NZW mice and 12-14 weeks for MLR/lpr mice. NZB×NZW mice with a proteinuria level greater than 100 mg/dL (around 27 weeks of age) were included in a study for evaluation of efficacy in advanced disease. Mice were continued to be monitored for the presence of proteinuria every 2-3 weeks for the duration of the study. Proteinuria was scored according to manufacturer instructions as follows: Trace: 0.5; ≥30 mg/dL: 1; ≥100 mg/dL: 2; ≥300 mg/dL: 3; ≥2000 mg/dL: 4.

### Mouse Dosing

Mice were injected with mIL-2/CD25 subcutaneously (s.c.) twice a week with a dosing volume of 200 μL per injection in a PBS vehicle. Mice given prednisolone were dosed 10 mg/kg three times a week orally (p.o.) with a dosing volume of 10 mL/kg, dissolved in water.

### Serum Antibody Titers

Mice were anesthetized with isoflurane and bled every 2-3 weeks during the study. At completion of study, serum was tested for the presence of anti-dsDNA autoantibodies by ELISA. Pooled serum from MRL/lpr mice with advanced lupus was used as a positive comparator in each assay. Autoantibody levels were quantified in arbitrary units based on stand curve generated with the positive control serum. IL-12p40 serum protein levels were measured from the serum taken at the end of 10 weeks of mIL-2/CD25 dosing, using an IL-12p40 ELISA kit from BD Biosciences according to the manufacturer's instructions.

### Histology

Upon termination of the study, one kidney from each animal was excised, immersion fixed in 10% NBF for 72 hours, and upon complete fixation, trimmed transversely, routinely processed paraffin embedded (RPPE) and sectioned at 4 m for H&E staining and 3 m for PASH staining. Slides were evaluated by a trained histopathologist in a single-blind manner for microscopic analysis of nephritis. Glomerular Nephritis (GN) and Tubulo-Interstitial Nephritis (TIN) were scored using a semi-quantitative 0-4 scale individually evaluating relevant pathologies. GN was scored for changes to the mesangium, cellular cast formation, mononuclear cell infiltration in glomerular tufts, and fibro-sclerosis of Bowman's capsule. TIN was scored for changes to tubularluminal infiltrate, tubular epithelial cell regeneration, protein casts, interstitial fibrosis, and mononuclear cells infiltration. The theoretical maximum total nephritis score was 36.

### Blood and Spleen Preparation and Staining

Blood samples were collected in heparin tubes and then immediately lysed and fixed with 1×BD FACS lysing solution for 10 min at 37° C. Cells were washed once with PBS then a second time with PBS containing 2% FBS, followed by permeabilization using ice-cold methanol for 30 minutes at 4° C. Samples were then washed twice to remove excess of methanol for staining.

Spleen samples, collected in PBS, were ground using the gentleMACS™ to prepare a single cell suspension, then filtered with 70 μM strainer. Using the ACK lysis buffer, red blood cells were lysed and the cell suspension was filtered once more with a 40 μM strainer. Cells were resuspended in PBS containing 2% FBS, and 1×10⁶ cells were directly fixed using 1.5% paraformaldehyde for 10 minutes at 37° C. Cells were washed with PBS containing 2% FBS followed by permeabilization using ice-cold methanol for 30 minutes at 4° C. Samples were then washed twice to remove excess methanol before staining.

Prepared blood or spleen samples were blocked using CD16/CD32 monoclonal antibody (BD Biosciences) follow by simultaneous stain with cell surface (CD4/CD8/CD25/CD335) and intracellular (Foxp3/Ki67/pSTAT5) markers for 45 minutes at 4° C. Samples were acquired using the BD Canto X flow cytometer and data analyzed using the FlowJo software (TreeStar).

### Kidney RT-PCR

At completion of study, one kidney from each mouse was collected in RNA Later and then homogenized in mRNA Catcher Lysis Buffer with a Tissue Lyser. mRNA was purified using mRNA Catcher PLUS according to the manufacturer's protocol (Invitrogen). cDNA was synthesized using SuperScript II with random hexamer primers. PCR was performed with SYBR Green Master Mix (Invitrogen). Relative quantification analysis was determined using the 2-ΔΔCT method using cyclophilin (PPIA) as the housekeeping gene. Expression of inflammatory cytokine and leukocyte surface receptors were analyzed.

Primer pairs:
Ifit1:
(SEQ ID NO: 35)
5′-AGAGCAGAGAGTCAAGGCAGGT-3′;
(SEQ ID NO: 36)
5′-TGGTCACCATCAGCATTCTCTCCCA-3′;
Ifit3:
(SEQ ID NO: 37)
5′-GCTCAGCCCACACCCAGCTTT-3′;
(SEQ ID NO: 38)
5′-AGATTCCCGGTTGACCTCACTCAT-3′;
Mx1:
(SEQ ID NO: 39)
5′-ACTACCAGGAGTGCAGACGGAA-3′;
(SEQ ID NO: 40)
5′-TCCTCCAGGAACCAGCTGCACTTA-3′;
Irf7:
(SEQ ID NO: 41)
5′-GAGTCTGGGGCAGACCCCGT-3′;
(SEQ ID NO: 42)
5′-CTGCGCTCGGTGAGAGCTGG-3′;
Gbp2:
(SEQ ID NO: 43)
5′-AGCTGCTAAACTTCGGGAACAGGA-3′;
(SEQ ID NO: 44)
5′-AGAGGTTTGGGCCTTGGGCCT-3′;
Ligp1:
(SEQ ID NO: 45)
540 -GGACACAGGAGTTTCTGTGCCTTT-3′;
(SEQ ID NO: 46)
5′-AGGTGAAGAGAACAGCTGACCCA-3′

### Pharmacokinetics

Serum levels of mIL-2/CD25 were determined in NZB×NZW F1 mice after the first dose (0.1, 0.2 or 0.4 mg/kg s.c.). Blood samples (0.1 mL) were obtained by submental bleeding at 24, 48, and 80 hours after the dose using composite sampling (4 mice per time point). Blood samples were allowed to coagulate and centrifuged at 4° C. (1500 to 2000×g) to obtain serum. Serum samples were stored at −80° C. until analysis by ligand binding assay on Chemiluminescence platform. Pharmacokinetic parameters (AUC, Cmax, Tmax and half-life) of mIL-2/CD25 were obtained by non-compartmental analysis of serum concentration vs. time data (Phoenix WinNonlin, Version 6.4, Certara USA, Inc., Princeton, NJ).

Ligand Binding Assay for Quantitation of mIL-2 CD25 in Serum

Samples, standards, and quality controls were brought up to a final matrix concentration of 33% mouse serum in PBS with 1% BSA. Briefly, 96 well black plates were coated with rat antimouse CD25 (eBioscience—Clone: PC61.5) at 1.0 μg/mL in PBS overnight at 4° C. The plates were blocked for 1 hour with PBS/Tween/20% casein prior to incubation with the serum for 2 hours at room temperature. mIL-2/CD25 was detected by sequential incubation with biotinylated rat anti-mouse IL-2 (eBioscience—Clone: JES6-SH4), NeutrAvidin—horseradish peroxidase (Thermo Scientific) and Pico Chemiluminescent substrate solution (Thermo Scientific). Plates were read in SpectraMax plate reader in luminescence mode. The concentration of mIL-2/CD25 in mouse serum was calculated from luminescence intensity using a Log-Log linear calibration curve (Softmax Analysis Program, Molecular Devices) generated from mIL-2/CD25 calibrators. The assay LLOQ was 25 μg/mL.

It is to be appreciated that the Detailed Description section, and not the Summary and Abstract sections, is intended to be used to interpret the claims. The Summary and Abstract sections may set forth one or more but not all exemplary aspects or embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects or embodiments will so fully reveal the general nature of the invention that others can, by applying knowledge within the skill of the art, readily modify and/or adapt for various applications such specific aspects or embodiments, without undue experimentation, without departing from the general concept of the present invention. Therefore, such adaptations and modifications are intended to be within the meaning and range of equivalents of the disclosed aspects or embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary aspects or embodiments, but should be defined only in accordance with the following claims and their equivalents.

The claims in the instant application are different than those of the parent application or other related applications. The Applicant therefore rescinds any disclaimer of claim scope made in the parent application or any predecessor application in relation to the instant application. The Examiner is therefore advised that any such previous disclaimer and the cited references that it was made to avoid, may need to be revisited. Further, the Examiner is also reminded that any disclaimer made in the instant application should not be read into or against the parent application.

1. A method of treating a disease or disorder in a subject in need thereof, comprising administering to the subject a dose of an Interleukin-2 (IL2) fusion protein, wherein the fusion protein comprises:
a first polypeptide comprising an IL2 polypeptide; and
a second polypeptide comprising an extracellular domain of an Interleukin-2 Receptor alpha (IL2Rα) polypeptide,
wherein (i) the extracellular domain of the IL2Rα polypeptide has at least one fewer glycosylation compared to the extracellular domain of native IL2Rα (SEQ ID NO: 1);
and/or (ii) the IL2 polypeptide has at least one fewer glycosylation compared to native IL2 (SEQ ID NO: 2); and
wherein the dose is from about 0.1 mg to about 9 mg.

2. The method of claim 1, wherein the fusion protein is administered to the subject via a topical, epidermal, mucosal, intranasal, oral, vaginal, rectal, sublingual, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural or intrasternal route.

3. The method of claim 2, wherein the fusion protein is administered to the subject via an intravenous route.

4. The method of claim 3, wherein the dose is between about 0.3 mg to about 9 mg, between about 3 mg to about 9 mg, between about 6 mg to about 9 mg, between about 0.1 mg to about 3 mg, between about 0.1 mg to about 1 mg, between about 0.1 mg to about 0.3 mg, between about 0.3 mg to about 6 mg, or between about 1 mg to about 3 mg.

5. The method of claim 3, wherein the dose is about 0.1 mg, about 0.3 mg, about 1 mg, about 2 mg, about 3 mg, about 4 mg, about 5 mg, about 6 mg, about 7 mg, about 8 mg, or about 9 mg.

6-12. (canceled)

13. The method of claim 2, wherein the fusion protein is administered to the subject via a subcutaneous route.

14. The method of claim 13, wherein the dose is between about 1 mg to about 8 mg, between about 3 mg to about 8 mg, between about 6 mg to about 8 mg, between about 1 mg to about 6 mg, between about 1 mg to about 3 mg, or between about 3 mg to about 6 mg.

15-19. (canceled)

20. The method of claim 13, wherein the dose is about 0.1 mg, about 0.3 mg, about 1 mg, about 2 mg, about 3 mg, about 4 mg, about 5 mg, about 6 mg, about 7 mg, or about 8 mg.

21. (canceled)

22. The method of claim 1, wherein two or more of the dose are administered at a dosing interval between two doses, wherein the dosing interval is at least about one day, at least about two days, at least about three days, at least about four days, at least about five days, at least about six days, at least about one week, at least about two weeks, at least about three weeks, at least about four weeks, at least about a month, at least about five weeks, at least about six weeks, at least about seven weeks, at least about eight weeks, at least about two months, at least about nine weeks, at least about ten weeks, at least about eleven weeks, at least about twelve weeks, or at least about three months.

23-24. (canceled)

25. The method of claim 22, wherein the dosing interval is about three weeks.

26. The method of claim 22, wherein the dosing interval is the same or different throughout the doses.

27. (canceled)

28. The method of claim 22, wherein at least one of the two or more doses is administered intravenously and at least one of the two or more doses is administered subcutaneously.

29-30. (canceled)

31. The method of any one of claim 1, wherein the disease or disorder comprises an infectious disease or an immune-mediated disease.

32. (canceled)

33. The method of claim 31, wherein the immune-mediated disease is selected from the group consisting of: type 1 diabetes; multiple sclerosis; rheumatoid arthritis; celiac disease; systemic lupus erythematosus; lupus nephritis; cutaneous lupus; juvenile idiopathic arthritis; Crohn's disease; ulcerative colitis; systemic sclerosis; graft versus host disease (GvHD); psoriasis; alopecia areata; HCV-induced vasculitis; Sjogren's syndrome; Pemphigus; Ankylosing Spondylitis; Behcet's Disease; Wegener's Granulomatosis; Takayasu's Disease; Autoimmune Hepatitis; Sclerosing Cholangitis; Gougerot-sjögren; inflammatory bowel disease; Immune Dysregulation, Polyendocrinopathy, Enteropathy, X-linked (IPEX) syndrome; and Macrophage Activation Syndrome.

34-35. (canceled)

36. The method of claim 31, further comprising administering to the subject a corticosteroid.

37. (canceled)

38. The method of claim 36, wherein: (a) the corticosteroid is administered to the subject via a topical, oral, intravenous, or intramuscular route; (b) the corticosteroid is administered before, concurrently with, or after each dose of the of the fusion protein; or (c) both (a) and (b).

39-40. (canceled)

41. The method of claim 1, wherein: (a) the extracellular domain of the IL2Rα polypeptide has at least one fewer glycosylation, at least two fewer glycosylations, at least three fewer glycosylations, at least four fewer glycosylations, at least five fewer glycosylations, at least six fewer glycosylations, at least seven fewer glycosylations, at least eight fewer glycosylations, or at least nine fewer glycosylations compared to the extracellular domain of native IL2Rα (SEQ ID NO: 1); (b) the IL2 polypeptide has at least one fewer glycosylation compared to native IL2 (SEQ ID NO: 2); or (c) both (a) and (b).

42. (canceled)

43. The method of claim 1, wherein: (a) the first polypeptide comprises an amino acid sequence at least about 60%, at least about 70%, at least about 80%, at least about 85%, at least about 90%, at least about 95%, at least about 96%, at least about 97%, at least about 98%, at least about 99% or about 100% identical to SEQ ID NO: 2; (b) the second polypeptide comprises an amino acid sequence at least about 60%, at least about 70%, at least about 80%, at least about 85%, at least about 90%, at least about 95%, at least about 96%, at least about 97%, at least about 98%, at least about 99%, or about 100% identical to SEQ ID NO: 3; or (c) both (a) and (b).

44-46. (canceled)

47. A method of treating a disease or disorder in a subject in need thereof, comprising administering to the subject a dose of an Interleukin-2 (IL2) fusion protein, wherein the fusion protein comprises:
a first polypeptide comprising an IL2 polypeptide; and
a second polypeptide comprising an extracellular domain of an Interleukin-2 Receptor alpha (IL2Rα) polypeptide,
wherein (i) the extracellular domain of the IL2Rα polypeptide has at least one fewer glycosylation compared to the extracellular domain of native IL2Rα (SEQ ID NO: 1);
and/or (ii) the IL2 polypeptide has at least one fewer glycosylation compared to native IL2 (SEQ ID NO: 2); and
wherein the dose is greater than about 9 mg.

48. A method of increasing a regulatory T cell activity in a subject in need thereof, comprising administering to the subject a dose of an Interleukin-2 (IL2) fusion protein, wherein the fusion protein comprises:
a first polypeptide comprising an IL2 polypeptide; and
a second polypeptide comprising an extracellular domain of an Interleukin-2 Receptor alpha (IL2Rα) polypeptide,
wherein (i) the extracellular domain of the IL2Rα polypeptide has at least one fewer glycosylation compared to the extracellular domain of native IL2Rα (SEQ ID NO: 1);
and/or (ii) the IL2 polypeptide has at least one fewer glycosylation compared to native IL2 (SEQ ID NO: 2); and
wherein the dose is from about 0.1 mg to about 9 mg.


SEQUENCE LISTING (46 sequences)

SEQ ID NO: 1 - protein, 219 aa, Artificial Sequence
  IL2Ralpha (human, mature form of IL2Ralpha extracellular domain)
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 2 - protein, 133 aa, Artificial Sequence [sequence omitted]

SEQ ID NO: 3 - protein, 191 aa, Artificial Sequence
  IL2Ralpha (human, mature form of IL2Ralpha extracellular domain) - half-truncated
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETS

SEQ ID NO: 4 - protein, 166 aa, Artificial Sequence
  IL2Ralpha (human, mature form of IL2Ralpha extracellular domain) - full-truncated
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGE

SEQ ID NO: 5 - protein, 336 aa, Artificial Sequence
  IL2-CD25(22-212)
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGSGGGSGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETS

SEQ ID NO: 6 - protein, 12 aa, Artificial Sequence
  glycine linker
GGGSGGGSGGGS

SEQ ID NO: 7 - protein, 153 aa, Artificial Sequence
  IL2 (human, unprocessed)
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 8 - protein, 149 aa, Artificial Sequence
  IL2 (mouse, mature form)
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQ

SEQ ID NO: 9 - protein, 169 aa, Artificial Sequence
  IL2 (mouse, unprocessed)
MYSMQLASCVTLTLVLLVNSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQ

SEQ ID NO: 10 - protein, 251 aa, Artificial Sequence
  IL2Ralpha (human, mature form)
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 11 - protein, 272 aa, Artificial Sequence
  IL2Ralpha (human, unprocessed form)
MDSYLLMWGLLTFIMVPGCQAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 12 - protein, 247 aa, Artificial Sequence
  IL2Ralpha (mouse, mature form)
ELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYKVAVASCLFLLISILLLSGLTWQHRWRKSRRTI

SEQ ID NO: 13 - protein, 268 aa, Artificial Sequence
  IL2Ralpha (mouse, unprocessed form)
MEPRLLMLGFLSLTIVPSCRAELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYKVAVASCLFLLISILLLSGLTWQHRWRKSRRTI

SEQ ID NO: 14 - protein, 219 aa, Artificial Sequence
  IL2Ralpha (mouse, mature form of IL2Ralpha extracellular domain)
ELCLYDPPEVPNATFKALSYKNGTILNCECKRGFRRLKELVYMRCLGNSWSSNCQCTSNILRASHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYK

SEQ ID NO: 15 - protein, 4 aa, Artificial Sequence
  Linker sequence
GGGS

SEQ ID NO: 16 - protein, 5 aa, Artificial Sequence
  glycine linker
GGGGS

SEQ ID NO: 17 - protein, 16 aa, Artificial Sequence
  glycine linker
GGGSGGGSGGGSGGGS

SEQ ID NO: 18 - protein, 20 aa, Artificial Sequence
  Glycine Linker
GGGSGGGSGGGSGGGSGGGS

SEQ ID NO: 19 - protein, 24 aa, Artificial Sequence
  Glycine Linker
GGGSGGGSGGGSGGGSGGGSGGGS

SEQ ID NO: 20 - protein, 28 aa, Artificial Sequence
  Glycine Linker
GGGSGGGSGGGSGGGSGGGSGGGSGGGS

SEQ ID NO: 21 - protein, 15 aa, Artificial Sequence
  Glycine Linker
GGGGSGGGGSGGGGS

SEQ ID NO: 22 - protein, 20 aa, Artificial Sequence
  Glycine Linker
GGGGSGGGGSGGGGSGGGGS

SEQ ID NO: 23 - protein, 25 aa, Artificial Sequence
  Glycine Linker
GGGGSGGGGSGGGGSGGGGSGGGGS

SEQ ID NO: 24 - protein, 10 aa, Artificial Sequence
  Glycine Linker
GGGGSGGGGS

SEQ ID NO: 25 - protein, 5 aa, Artificial Sequence
  Glycine Linker
GGGGS

SEQ ID NO: 26 - protein, 30 aa, Artificial Sequence
  Glycine Linker
GGGGSGGGGSGGGGSGGGGSGGGGSGGGGS

SEQ ID NO: 27 - protein, 35 aa, Artificial Sequence
  Glycine Linker
GGGGSGGGGSGGGGSGGGGSGGGGSGGGGSGGGGS

SEQ ID NO: 28 - protein, 25 aa, Artificial Sequence
  Glycine Linker
GGGGSGGGGSGGGGSGGGGSGGGGS

SEQ ID NO: 29 - protein, 364 aa, Artificial Sequence
  IL2(21-153)-(G3S)3-CD25(22-240)
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGSGGGSGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 30 - protein, 311 aa, Artificial Sequence
  IL2-CD25(22-187)
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGSGGGSGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGE

SEQ ID NO: 31 - protein, 331 aa, Artificial Sequence
  NativeSigPep-IL2-CD25(22-187)
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGSGGGSGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGE

SEQ ID NO: 32 - protein, 358 aa, Artificial Sequence
  NativeSigPep-HuIL2-CD25(22-212)-PP
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGSGGGSGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSPP

SEQ ID NO: 33 - protein, 311 aa, Artificial Sequence; identical to SEQ ID NO: 30
  IL2-CD25(22-187)

SEQ ID NO: 34 - protein, 311 aa, Artificial Sequence
  IL2-T23A-C145S-CD25(22-187)
APASSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTGGGSGGGSGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGE

SEQ ID NO: 35 - DNA, 22 bp, Artificial Sequence
  Primer Ifit1
agagcagagagtcaaggcaggt

SEQ ID NO: 36 - DNA, 25 bp, Artificial Sequence
  Primer Ifit1
tggtcaccatcagcattctctccca

SEQ ID NO: 37 - DNA, 21 bp, Artificial Sequence
  Primer Ifit3
gctcagcccacacccagcttt

SEQ ID NO: 38 - DNA, 24 bp, Artificial Sequence
  Primer Ifit3
agattcccggttgacctcactcat

SEQ ID NO: 39 - DNA, 22 bp, Artificial Sequence
  Primer Mx1
actaccaggagtgcagacggaa

SEQ ID NO: 40 - DNA, 24 bp, Artificial Sequence
  Primer Mx1
tcctccaggaaccagctgcactta

SEQ ID NO: 41 - DNA, 20 bp, Artificial Sequence
  Primer Irf7
gagtctggggcagaccccgt

SEQ ID NO: 42 - DNA, 20 bp, Artificial Sequence
  Primer Irf7
ctgcgctcggtgagagctgg

SEQ ID NO: 43 - DNA, 24 bp, Artificial Sequence
  Gbp2
agctgctaaacttcgggaacagga

SEQ ID NO: 44 - DNA, 21 bp, Artificial Sequence
  Primer Gbp2
agaggtttgggccttgggcct

SEQ ID NO: 45 - DNA, 24 bp, Artificial Sequence
  Primer Ligp1
ggacacaggagtttctgtgccttt

SEQ ID NO: 46 - DNA, 23 bp, Artificial Sequence
  Primer Ligp1
aggtgaagagaacagctgaccca